(19) United States
(12) Patent Application Publication
(10) Pub. No.: US 2005/0074793 A1
(43) Pub. Date: Apr. 7, 2005

(54) Metastatic colorectal cancer signatures

(76) Inventors: Keith E. Wilson, Redwood City, CA (US); J. Sunil Rao, Richmond Heights, OH (US); Sandy Markowitz, Pepper Pike, OH (US); Ghassan Ghandour, Atherton, CA (US)

Correspondence Address: HOWREY SIMON ARNOLD & WHITE, LLP, c/o IP DOCKETING DEPARTMENT, 2941 FAIRVIEW PARK DRIVE, SUITE 200, FALLS CHURCH, VA, 22042-2924, US

(21) Appl. No.: 10/818,066

(22) Filed: Apr. 2, 2004

Related U.S. Application Data
(60) Provisional application No. 60/460,892, filed on Apr. 4, 2003.

(51) Int. Cl. C12Q 1/68; G01N 33/53; G06F 19/00; G01N 33/48; G01N 33/50; G01N 33/574

(52) U.S. Cl. 435/6; 435/7.1; 702/19; 435/7.23

(57) ABSTRACT

The present invention provides defined sets of genes that are used for identification and diagnosis of metastatic cancer and other conditions in a biological sample. The defined sets of genes can also be used for prognosis evaluation of a patient based on the gene expression pattern of a biological sample.

### REFERENCE TO RELATED APPLICATIONS

This application claims priority from U.S. Provisional Application No. 60/460,892 filed Apr. 4, 2003, which is hereby incorporated by reference herein in its entirety.

This invention was made at least in part with assistance from the United States Federal Government, under Grant No. U01 CA88130 from the National Institutes of Health. As a result, the government may have certain rights to this invention.

### BACKGROUND OF THE INVENTION

Cancer of the colon and/or rectum (referred to as “colorectal cancer”) is significant in Western populations, particularly in the United States. Cancers of the colon and rectum occur in both men and women, most commonly after the age of 50. Colorectal cancer is the second leading cancer killer in the United States, and the third most common cancer overall. This year, more than 50,000 Americans will die from colorectal cancer and approximately 131,600 new cases will be diagnosed.

Mutations in tumor-suppressor genes, proto-oncogenes, and DNA repair genes are factors known to influence the development of tumorigenesis. For example, inactivating both alleles of the adenomatous polyposis coli (APC) gene, a tumor suppressor gene, appears to be one of the earliest events in colorectal cancer, and may even be the initiating event. Other genes implicated in colorectal cancer include the MCC gene, the p53 gene, the DCC (deleted in colorectal carcinoma) gene and other chromosome 18q genes, and genes in the TGF-β signaling pathway (for a review, see Molecular Biology of Colorectal Cancer, pp. 238-299, in Curr. Probl. Cancer, September/October 1997; see also Willams, Colorectal Cancer (1996); Kinsella & Schofield, Colorectal Cancer: A Scientific Perspective (1993); Colorectal Cancer: Molecular Mechanisms, Premalignant State and its Prevention Schmiegel & Scholmerich eds., 2000; Colorectal Cancer: New Aspects of Molecular Biology and Their Clinical Applications (Hanski et al., eds 2000); McArdle et al., Colorectal Cancer (2000); Wanebo, Colorectal Cancer (1993); Levin, The American Cancer Society: Colorectal Cancer (1999); Treatment of Hepatic Metastases of Colorectal Cancer (Nordlinger & Jaeck eds., 1993); Management of Colorectal Cancer (Dunitz et al., eds. 1998); Cancer: Principles and Practice of Oncology (Devita et al., eds. 2001); Surgical Oncology: Contemporary Principles and Practice (Kirby et al., eds. 2001); Offit, Clinical Cancer Genetics: Risk Counseling and Management (1997); Radioimmunotherapy of Cancer (Abrams & Fritzberg eds. 2000); Fleming, AJCC Cancer Staging Handbook (1998); Textbook of Radiation Oncology (Leibel & Phillips eds. 2000); and Clinical Oncology (Abeloff et al., eds. 2000).

As with all cancers, there are stages of disease progression, as well as expected survival rates for these different stages. The American Cancer Society reports that the 5-year relative survival rate is 90% for people whose colorectal cancer is treated in an early stage, before it has spread. But, only 37% of colorectal cancers are found at that early stage. Once the cancer has spread to nearby organs or lymph nodes, the 5-year relative survival rate goes down to 65%. For people whose colorectal cancer has spread to distant parts of the body such as the liver or lungs, the 5-year relative survival rate is 9%. Thus, metastasis of the tumor to the liver lungs and regional lymph nodes are important prognostic factors (see, e.g., PET in Oncology: Basics and Clinical Application (Ruhlmann et al. eds. 1999).

Since tumor metastases is the principal cause of death for cancer patients, a better understanding of the various factors involved in this process, especially about the gene expression exhibited by these cancers, will have prognostic and diagnostic value. Indeed, patterns of gene expression associated with the various stages of these cancers would provide an important tool in the selection of treatment alternatives.

Comparing the gene expression profiles of different cells and tissues can provide information about the identity of the tissue, the health status of the tissue and other properties. For example, genes that are differentially expressed in healthy and pathologic cells can function as diagnostic markers. Additionally, such genes are candidate targets for regulation by therapeutic intervention.

There are numerous methods presently in use for generating gene expression profiles of a cell or tissue. However, there remains a need in the art for methods that utilize the information embodied in a gene expression profile for the benefit of diagnosing, treating or determining the probable prognosis of disease.

Accordingly, provided herein are methods that can be used in diagnosis and prognosis evaluation of metastatic colorectal cancer. Further provided are methods that can be used to screen candidate therapeutic agents for the ability to modulate, e.g., treat, colorectal cancer. Additionally, provided herein are molecular targets and compositions for therapeutic intervention in metastatic colorectal disease and other metastatic cancers.

### BRIEF SUMMARY OF THE INVENTION

The present invention provides materials and methods for characterizing biological samples, thereby providing diagnostic methods for identifying cells and tissues and evaluating their physiological status. The methods involve obtaining a biological sample, generating a gene expression profile of the biological sample, and comparing the gene expression profile of a select group of genes from the biological sample with gene expression profile represented by the reference sets of the Tables 1-6.

The select groups of genes used for comparison, identification, and diagnosis of the health status of a biological sample comprise the reference sets of the Tables 1-6. The reference sets of the Tables 1-6 comprise genes selected for their high signal-to-noise ratio in reference samples. These genes, herein referred to as “classifier genes” provide maximum information regarding the nature and identity of a given biological sample.

In one aspect the invention provides a method of diagnosing the health status of a biological sample comprising the steps of; generating a gene expression pattern of the biological sample, and comparing the gene expression pattern of the biological sample with the reference sets of the Tables 1-6, wherein a match between the gene expression pattern of one or more genes in the biological sample and one or more genes of the Tables 1-6 provides a diagnosis of the biological sample. In one embodiment, the biological sample comprises cells obtained from a biopsy sample. In another embodiment, the biological sample is diagnosed as healthy tissue. In yet another embodiment, the biological sample is diagnosed as having metastatic colorectal cancer.

In one embodiment analysis of the gene expression pattern of the biological sample indicates that the colon cancer is likely to develop future metastasis.

In one embodiment, the diagnosis of the biological sample is made with reference to at least five different classifier genes from Tables 1-6.

In another embodiment, comparison of the gene expression pattern of the biological sample and the reference sets identifies the tissue origin of the metastatic cancer.

In one embodiment, the comparison of the gene expression pattern of the biological sample and the reference sets is made by comparing RNA expression profiles.

In another embodiment, the comparison of the gene expression pattern of the biological sample and the reference sets is made by comparing protein expression profiles.

In one embodiment, the protein expression profile is evaluated using antibodies.

In one aspect, the invention provides a method for prognosis evaluation of metastatic colorectal cancer comprising the steps of; generating a gene expression pattern of the biological sample, and comparing the gene expression pattern of the biological sample with the reference sets of the Tables 1-6, wherein a match between the gene expression pattern of the biological sample and one or more reference sets provides a prognosis evaluation of the metastatic potential of the colorectal cancer. In one embodiment, a match between the gene expression pattern of the biological sample and the reference set representing colon cancer hepatic metastases is indicative of poor prognosis.

In another aspect the invention provides a method for evaluating the progress of treatment of metastatic colorectal cancer comprising the steps of; generating a first gene expression pattern of a first biological sample from a patient, comparing the first gene expression pattern of the first biological sample with the reference sets of the Tables 1-6, obtaining a match between the first gene expression pattern of the first biological sample and one or more reference sets of the Tables 1-6, thereby providing an initial diagnosis of metastatic colorectal cancer, then administering to the patient a therapeutically effective amount of a compound that modulates the metastatic colorectal cancer, generating a second gene expression profile of a second biological sample from the patient, and comparing the second gene expression pattern of the second biological sample with the reference sets of the Tables 1-6, then comparing the match between the second gene expression pattern of the second biological sample and the match between the first gene expression pattern of the first biological sample wherein the comparison indicates the progress of the treatment for metastatic colorectal cancer.

In another aspect, the invention provides a method for evaluating the efficacy of drug candidates for the treatment of metastatic colorectal cancer, comprising the steps of; contacting a cell or tissue culture that has a gene expression profile indicative of metastatic colorectal cancer with an effective amount of a test compound, generating a gene expression profile of the contacted cell or tissue culture, and comparing the gene expression pattern of the contacted cell culture with the defined sets of genes of the Tables 1-6, obtaining a match between the gene expression pattern of the contacted cell culture and thereby determining the efficacy of the drug compound for the treatment of metastatic colorectal cancer.

In another aspect, the invention provides a kit for identifying the gene expression pattern of a biological sample comprising; nucleic acid probes that specifically bind to nucleotide sequences from reference sets of the Tables 1-6, and means of labeling nucleic acids. In one embodiment the kit comprises nucleic acid probes that identify metastatic cancer derived from a primary tumor in an organ selected from the group consisting of heart, lung, pancreas, breast, prostate, and colon.

In another aspect, the invention provides a kit for identifying the gene expression pattern of a biological sample comprising; antibodies or ligands that specifically bind to polypeptides encoded by a genes of the reference sets of the Tables 1-6, and means of labeling the antibodies or ligands that specifically bind to polypeptides encoded by genes of the reference sets of the Tables 1-6. In one aspect, the kit provides antibodies or ligands that identify metastatic cancer derived from a primary tumor in an organ selected from the group consisting of lung, pancreas, breast, prostate, and colon.

### DETAILED DESCRIPTION OF THE INVENTION

Definitions

By “metastatic colorectal cancer” herein is meant a colon and/or rectal tumor or cancer that is classified as Dukes stage C or D (see, e.g., Cohen et al., Cancer of the Colon, in Cancer: Principles and Practice of Oncology, pp. 1144-1197 (Devita et al., eds., 5^{th} ed. 1997); see also Harrison's Principles of Internal Medicine, pp. 1289-129 (Wilson et al., eds., 12^{th} ed., 1991). “Treatment, monitoring, detection or modulation of metastatic colorectal cancer” includes treatment, monitoring, detection, or modulation of metastatic colorectal disease in those patients who have metastatic colorectal disease (Dukes stage C or D). In Dukes stage A, the tumor has penetrated into, but not through, the bowel wall. In Dukes stage B, the tumor has penetrated through the bowel wall but there is not yet any lymph involvement. In Dukes stage C, the cancer involves regional lymph nodes. In Dukes stage D, there is distant metastasis, e.g., liver, lung, etc.

The term “metastasis” refers to the process by which a disease shifts from one part of the body to another. This process may include the spreading of neoplasms from the site of a primary tumor to distant parts of the body.

The term “metastatic cancer” refers to any cancer in any part of the body which has its origins in primary cancer at a site distant from the location of the secondary tumor. Metastatic cancer includes, but is not limited to true “metastatic tumors” as well as pre-metastatic primary tumor cells in the process of developing a metastatic phenotype.

The term “metastatic potential” refers to the like hood that a particular tumor will metastasize. A tumor with metastatic potential has a high likelihood of progressing to metastatic cancer.

The term “secondary tumor” refers to a metastatic tumor that has developed at a site distant from the location of the original, primary cancer.

“Classifier genes” are genes selected for the purpose of comparison and identification of biological samples. Classifier genes are selected by virtue of the high signal-to-noise ratio and reproducibility they display when measured in reference samples. Classifier genes are considered “maximally informative genes” because the ability to clearly and reliably detect them provides maximum information regarding the nature and identity of a given biological sample.

A specific classifier gene may or may not be uniquely expressed in a particular cell, tissue, or organ. In some applications, the classifier gene may be tissue-specific; that is, expressed exclusively in a particular tissue or cell type. In other applications the classifier gene may be expressed predominantly in one tissue type, but could also be expressed in other cells, tissues or organs, but in a different relationship with the other classifier genes of the set. Thus, the level of expression of a classifier gene, and its relationship within a pattern of co-expressed genes creates a unique profile that can be used to infer the identity and physiology of an unknown biological sample.

Classifier genes may encode intracellular molecules, e.g., cellular nucleic acids, intracellular proteins, and the intracellular domains of transmembrane proteins, or extracellular molecules such as the extracellular domains of transmembrane proteins or secreted proteins. Intracellular and extracellular classifier molecules are equally suitable.

The protein product of a classifier gene may be referred to herein as a “classifier protein”. Similarly, “classifier molecule” may be used herein to refer collectively to both classifier genes and classifier proteins.

Subsets of classifier genes representative of the gene expression patterns of different cells, tissues, organs and physiological states of disease and health are organized into the reference sets of the Tables 1-6.

The term “metastatic colorectal cancer classifier protein” or “metastatic colorectal cancer classifier polynucleotide” or “metastatic colorectal cancer classifier gene sequences” refers to nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a UniGene cluster of Tables 1-6; (2) bind to antibodies, e.g., polyclonal antibodies, raised against an immunogen comprising an amino acid sequence encoded by a nucleotide sequence of or associated with a UniGene cluster of Tables 1-6, and conservatively modified variants thereof; (3) specifically hybridize under stringent hybridization conditions to a nucleic acid sequence, or the complement thereof of Tables 1-6 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a UniGene cluster of Tables 1-6. A polynucleotide or polypeptide sequence is typically from a mammal including, but not limited to, primate, e.g., human; rodent, e.g., rat, mouse, hamster; cow, pig, horse, sheep, or other mammal. A “metastatic colorectal cancer classifier gene sequence” a includes both naturally occurring or recombinant nucleotide and protein sequences.

“Reference set” refers to defined sets of classifier genes that characterize a particular tissue, organ, cell, cell culture or physiological state of a biological sample. The reference set may form part of an organized hierarchical structure for the classification of individual tissues or organs. If the reference set is part of an organized hierarchical structure, it may be used to identify or distinguish a sample at either the highest or lowest level of classification, or it may contain defined sets of genes representing one or more levels of classification for a given tissue or organ and therefore use several levels simultaneously to identify a sample.

Table 1 illustrates the hierarchical structure of classification that orders the defined sets of classifier genes comprising the reference sets of the invention. These defined sets of classifier genes can be used to characterize individual tissues and organs from humans. The defined sets of genes are organized hierarchically to permit identification of a sample on several levels of detail. For example, using the reference sets of classifier genes of Tables 1-6, it is possible to determine that a sample comprises adipose tissue. Within the context of this reference set that identifies adipose tissue, further analysis could reveal other defined sets of classifier genes which, when compared to the reference sets of classifier genes in Tables 1-6 identify the sample as being mammary tissue as opposed to omental tissue or simple adipose tissue. The sample could be still further analyzed within the context of the reference set that characterizes adipose tissue, to determine that the sample is a sample of breast tissue.

A “signature” refers to a specific pattern of gene expression as reflected in a particular defined set of classifier genes of the Tables 1-6. The “signature” of a biological sample is a unique identifier of the sample.

A “tissue” refers to a complex, integrated group of cohesive, typically spatially aggregated cells; certain “tissues” are disperse, e.g., blood cells or skin that share a common structure and/or function. Alternatively, complex assemblies of tissues form functional systems of organs. See, e.g., Rohen, et al. (2002) Color Atlas of Anatomy: A Photographic Study of the Human Body Lippincott; Hiatt, et al. (2000) Color Atlas of Histology Lippincott.

“Biological sample” refers to a sample derived from a virus, cell, tissue, organ, or organism including, without limitation, cell, tissue or organ lysates or homogenates, or body fluid samples, such as blood, urine, sputum, or cerebrospinal fluid. Such samples include, but are not limited to, tissue isolated from humans, or explants, primary, and transformed cell cultures derived therefrom. Biological samples may also include sections of tissues such as frozen sections taken for histologic purposes. A biological sample can be obtained from a eukaryotic organism such as fungi, plants, insects, protozoa, birds, fish, reptiles, and preferably a mammal such as rat, mouse, cow, dog, guinea pig, or rabbit, and most preferably a primate such as cynomologous monkeys, rhesus monkeys, chimpanzees, or humans.

“Encoding” refers to the property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. A gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. See, e.g., Lodish, et al. (2000) Mol. Cell Biol. (4th ed.) Freeman; Alberts, et al. (1994) Mol. Biol. Cell Garland.

“Differential expression” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus metastatic colorectal cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection.

A component of a biological sample is differentially expressed between two samples if the difference in amount of the component in one sample vs. the amount in the other sample is statistically significant. For example, preferably the change in expression (i.e., upregulation or downregulation) is typically at least about 50%, more preferably at least about 100%, more preferably at least about 150%, more preferably at least 180%, 200%, 300%, 500%, 700%, 900%, or 1000% the amount in the other sample, or if it is detectable in one sample and not detectable in the other.

“Gene expression profile” refers to the identification of at least one mRNA or protein expressed in a biological sample.

“Nucleic acid array” refers to an array of addressable locations (e.g., a location characterized by a distinctive, interrogatable address), each addressable location comprising a characteristic nucleic acid attached thereto. A nucleic acid as defined herein, may be a naturally occurring or synthetic nucleic acid, e.g., an oligonucleotide or polynucleotide. In an oligonucleotide array, the nucleic acid is an oligonucleotide (e.g., corresponding to an exon, EST, or a portion of a gene, transcript, or cDNA); in an EST array the nucleic acid is an EST or portion thereof; in an mRNA array the nucleic acid is an mRNA or portion thereof, or a corresponding cDNA. An oligonucleotide can be from 4, 6, 8, 10, or 12 nucleotides or longer in length, often 10, 30, 40, or 50 nucleotides in length, up to about 100 nucleotides in length. See Kohane, et al. (2002) Microarrays for Integrative Genomics MIT Press; Baldi and Hatfield (2002) DNA Microarrays and Gene Expression Cambridge Univ. Press.

“Detect” refers to identifying the presence, absence or amount of the object to be detected. “Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. “Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. See Paul (1999) Fundamental Immunology (4th ed.) Raven.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 4th ed. 1999)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv, diabodies [dimers of scFv], minibodies [scFv-CH3 fusion proteins]) or those identified using phage display libraries (see, e.g., McCafferty et al., Nature 348:552-554 (1990)).

Monoclonal or polyclonal antibodies my be prepared by many techniques. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens. See, e.g., McCafferty et al., Nature 348:552-554 (1990); Marks et al., Biotechnology 10:779-783 (1992).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, and/or quantify the antigen. See Coligan, et al. (1993 and supplements) Current Protocols in Immunology Wiley.

When used in the context of an antibody-antigen reaction, “specific” or “selective binding” of an antibody refers to a binding reaction that is determinative of the presence of the antigen in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a polypeptide encoded by a polynucleotide of Tables 2-5, or splice variants, or portions thereof, can be selected to obtain only those polyclonal antibodies that are specifically immunoreactive with the selected polypeptide and not with other proteins. Where the target protein is a member of a family such as GPCRs, this selection may be achieved by subtracting out antibodies that cross-react with molecules such as other GPCR family members. In addition, polyclonal antibodies raised to target polymorphic variants, alleles, orthologs, and conservatively modified variants can be selected to obtain only those antibodies that recognize the target protein, but not other GPCR family members. In addition, antibodies reactive to human target proteins but not homologs from other species can be selected in the same manner. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Using Antibodies: A Laboratory Manual, New York: Cold Spring Harbor Laboratory Press (1998). for a description of immunoassay formats and conditions that can be used to determine specific immunoreactivity).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of Tables 2-6 encoding a polypeptide is separated from open reading frames that flank the polypeptide coding sequence gene and encode proteins other than the polypeptide of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure. See, e.g., Walsh (2002) Proteins: Biochemistry and Biotechnology Wiley; Hardin, et al. (eds. 2001) Cloning, Gene Expression and Protein Purification Oxford Univ. Press; Wilson, et al. (eds. 2000) Encyclopedia of Separation Science Academic Press.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequencesin which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analog refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: Alanine (A), Glycine (G); Aspartic acid (D), Glutamic acid (E); Asparagine (N), Glutamine (Q); Arginine (R), Lysine (K); Isoleucine (I), Leucine (L), Methionine (M), Valine (V); Phenylalanine (F), Tyrosine (Y), Tryptophan (W); Serine (S), Threonine (T); and Cysteine (C), Methionine (M). See, e.g., Creighton, Proteins (1984) Freeman).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. See Ausubel (ed. 1993) Current Protocols in Molecular Biology Wiley.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. See, e.g., Lodish, et al. (2000) Mol. Cell Biol. (4th ed.) Freeman; Alberts, et al. (1994) Mol. Biol. Cell Garland.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “identify” in the context of the invention means to be able to recognize a particular gene expression pattern as being characteristic of a particular cell, tissue, organ, physiological state, or in the case of testing for compatibility of transplant donors and recipients the gene expression pattern may be characteristic of a particular individual.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to a nucleotide sequence such as those of Tables 2-5, or to an amino acid sequence encoded by a polynucleotide of Tables 2-5, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length or larger, e.g., 200-500 or more. See, e.g., Baxevanis, et al. (2001) Bioinformatics: A Practical Guide to the Analysis of Genes and Proteins Wiley; Mount (2000) Bioinformatics: Sequence and Genome Analysis CSH Press; Ewens and Grant (2001) Statistical Methods in Bioinformatics: An Introduction Springer-Verlag; Sensen (ed. 2002) Essentials of Genomics and Bioinformatics Wiley.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 2001 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA). See, e.g., Andersen (1998) Nucleic Acid Hybridization Springer-Verlag; Ross (ed. 1997) Nucleic Acid Hybridization Wiley.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5× SSC and 1% SDS incubated at 42° C. or 5× SSC and 1% SDS incubated at 65° C., with a wash in 0.2×SSC and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 sec, an annealing phase lasting 30-120 sec., and an extension phase of about 72° C. for 1-2 min.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency.

Introduction

In accordance with the objects outlined above, the present invention provides materials and methods for characterizing the nature of biological samples, thereby permitting one to identify a biological sample and/or evaluate its physiological state. In particular, the invention provides novel methods for diagnosis and treatment of colon and/or rectal cancer (e.g., colorectal cancer), including metastatic colorectal cancers, as well as methods for screening for compositions which modulate colorectal cancer. The method is also useful for differentiating between particular stages of cancer, for example Duke's stage A, B, C, or D colorectal cancers. The method is also effective for determining the origin of metastatic cancer.

The methods of the present invention allow one to compare a set of genes expressed in a biological sample with reference set, and to thereby identify a cell culture, tissue or organ from which a biological sample is derived. Alternatively, the comparison may yield information useful for diagnosing the health status of tissue or organ sample. In some embodiments the invention is permits the prognosis evaluation of a patient with cancer, particularly colorectal cancer. In other embodiments the invention provides a method for monitoring the progress of therapeutic intervention to cure metastatic colorectal cancer.

The invention comprises reference sets of classifier genes whose characteristic patterns of expression can be used to determine the physiological state of a biological sample. The genes comprising the reference sets are selected for their high signal to noise ratio in a reference sample. These genes are considered “maximally informative genes” or “classifier genes”. Any particular classifier gene of a reference set may or may not be uniquely expressed in a particular biological sample. However, the level of expression of such a gene, and its relationship within a pattern of co-expressed genes creates a unique profile that can be used to infer the identity and/or physiology of a biological sample. Reference sets, representing the gene expression pattern characteristic of metastatic tumors or tumors with metastatic potential are shown in the Tables 1-6. The genes indicative of a tumor with metastatic potential, may be either up-regulated or down-regulated with respect to samples from tumor or tissue that does not show metastatic potential.

Classifier genes may be a portion of a larger polynucleotide comprising a polynucleotide as shown in the Tables 1-6 (e.g., a full length mRNA or cDNA). Alternatively classifier genes may be a portion of a polypeptide encoded by a larger polynucleotide comprising a polynucleotide as shown in the Tables 1-6. “Genes” in this context includes coding regions, non-coding regions, and mixtures of coding and non-coding regions. Accordingly, as will be appreciated by those in the art, using the sequences provided herein, extended sequences, in either direction, of the metastatic colorectal cancer genes can be obtained, using techniques well known in the art for cloning either longer sequences or the full length sequences; see Current Protocols in Molecular Biology (Ausubel et al., eds., 1994). Selection of an appropriate portion of a polynucleotide for sequence hybridization, or of an appropriate portion of a polypeptide for immunological or other recognition, is dictated by optimal hybridization or immunogenicity and may be accomplished by the methods described herein e.g. microarray techniques.

Selection of the classifier polynucleotide or polypeptide is in accordance with the particular analysis to which the biological sample will be subjected. A general property of classifier genes and their corresponding polypeptides is that expression of defined sets of classifier genes can be compared with the reference sets of the Tables 1-6 to determine the metastatic potential of a biological sample. In some applications, it is desirable for the classifier gene to be tissue-specific or disease-specific that is, expressed exclusively in the tissue, cells or disease of interest. In other applications, the classifier gene may be expressed predominantly in one tissue type, or disease state, but could also be expressed in other tissues, or in a healthy state, but in a different relationship with the other classifier genes of the set. For example, a particular classifier gene may be expressed at different levels in biological sample comprising a colon liver metastasis, compared to a non-metastatic colon cancer (e.g. Duke's stage B colorectal cancer that was cured by surgery).

Classifier genes may encode either intracellular molecules e.g., cellular nucleic acids, intracellular proteins, and the intracellular domains of transmembrane proteins, or may encode extracellular molecules, such as the extracellular domains of transmembrane proteins. Intracellular and extracellular classifier genes are equally suitable.

Protein expression patterns may be evaluated by methods other than hybridization or antibody based detection. For example: chromatographic separation of proteins; ELISA or Ab based separations; affinity chromatography, 2d gels; general protein separation methods with analysis of individual “classifier” proteins all may be used (Padzikill (2002) Proteomics Kluwer; Liebler (2001) Introduction to Proteomics: Tools for the New Biology Humana; Suhai (ed. 2000) Genomics and Proteomics: Functional and Computational Aspects Kluwer; Rabilloud (ed. 2001) Proteome Research: Two Dimensional Gel Electrophoresis and Detection Methods Springer-Verlag; Hames and Rickwood (eds. 2001) Gel Electrophoresis of Proteins: A Practical Approach Oxford Univ. Press; James (ed. 2000) Proteome Research: Mass Spectrometry Springer-Verlag; Kyriakidis, et al. (eds. 2001) Proteome and Protein Analysis Springer-Verlag.)

Gene Expression Profiling

A first step in the methods of the invention is performing gene expression profiling of a sample of interest. Gene expression profiling refers to examining expression of one or more RNAs or proteins in a cell or tissue. Often at least or up to 10, 100, 1000, 10,000 or more different RNAs or proteins are examined in a single experiment. The profile of the sample is the compared with the reference sets of the Tables 1-6. In some embodiments, a given classifier gene may have a similar expression pattern in different cells. In other embodiments, the gene of interest may have lower or higher expression in one cell, tissue, organ or physiological state as compared to another.

The evaluating assays of the invention may be of any type. High-density expression arrays can be used, but other techniques are also contemplated. Methods for examining gene expression, often but not always hybridization based, include, e.g., Northern blots; dot blots; primer extension; nuclease protection; subtractive hybridization and isolation of non-duplexed molecules using, e.g., hydroxyapatite; solution hybridization; filter hybridization; amplification techniques such as RT-PCR and other PCR-related techniques such as differential display, LCR, AFLP, RAP, etc. (see, e.g., U.S. Pat. Nos. 4,683,195 and 4,683,202; PCR Protocols: A Guide to Methods and Applications (Innis et al., eds, 1990); Liang & Pardee, Science 257:967-971 (1992); Hubank & Schatz, Nuc. Acids Res. 22:5640-5648 (1994); Perucho et al., Methods Enzymol. 254:275-290 (1995)), fingerprinting, e.g., with restriction endonucleases (Ivanova et al., Nuc. Acids. Res. 23:2954-2958 (1995); Kato, Nuc. Acids Res. 23:3685-3690 (1995); and Shimkets et al., Nature Biotechnology 17:798-803, see also U.S. Pat. No. 5,871,697)); and the use of structure specific endonucleases (see, e.g., De Francesco, The Scientist 12:16 (1998)). mRNA expression can also be analyzed using mass spectrometry techniques (e.g., MALDI or SELDI), liquid chromatography, and capillary gel electrophoresis, as described below.

For a general description of these techniques, see also Sambrook et al., Molecular Cloning, A Laboratory Manual (2nd ed. 1989), see, e.g., pages 7.37-7.39, 7.53-7.54, 7.58-7.66, and 7.71-7.79; Kriegler, Gene Transfer and Expression: A Laboratory Manual (1990); and Current Protocols in Molecular Biology (Ausubel et al., eds., 1994).

Techniques have been developed that expedite expression analysis and sequencing of large numbers of nucleic acids samples. For example, nucleic acid arrays have been developed for high density and high throughput expression analysis (see, e.g., Granjeuad et al., BioEssays 21:781-790 (1999); Lockhart & Winzeler, Nature 405:827-836 (2000)). Nucleic acid arrays refer to large numbers (e.g., tens, hundreds, thousands, tens of thousands, or more) of different nucleic acid probes bound to solid substrates, such as nylon, glass, or silicon wafers (see, e.g., Fodor et al., Science 251:767-773 (1991); Brown & Botstein, Nature Genet. 21:33-37 (1999); Eberwine, Biotechniques 20:584-591 (1996)). A single array can contain probes corresponding to an entire genome, to all genes expressed by the genome, or to a selected subset of genes. The probes on the array can be DNA oligonucleotide arrays (e.g., GeneChip®, see, e.g., Lipshutz et al., Nat. Genet. 21:20-24 (1999)), mRNA arrays, cDNA arrays, EST arrays, or optically encoded arrays on fiber optic bundles (e.g., BeadArray™). The samples applied to the arrays for expression analysis can be, e.g., PCR products, cDNA, mRNA, etc.

Additional techniques for rapid gene sequencing and analysis of gene expression include, for example, SAGE (serial analysis of gene expression). For SAGE, a short segment of the original transcript (typically about 14 bp) is cleaved from the transcript for analysis. This sequence contains sufficient information to uniquely identify a transcript, and is referred to as a sequence tag. Sequence tags are collected from all the mRNA transcripts of a sample by binding of the poly-A tail of the mRNAs to a poly-T column. The sequence tags are linked together to form long concatameric molecules that are cloned, amplified, and sequenced. Analysis of the resulting sequence data will identify each transcript and reveal the number of times a particular tag is observed. Thus the method permits the expression level of the corresponding transcript to be determined (see, e.g., Velculescu et al., Science 270:484-487 (1995); Velculescu et al., Cell 88 (1997); and de Waard et al., Gene 226:1-8 (1999)).

Embodiments of the Invention

As described herein, each of these techniques can be used, alone or in combination, to identify a classifier gene or set of classifier genes expressed in a cell, tissue organ or disease state. Classifier genes may encode, for example, ion channels, receptors, G protein coupled receptors, cytokines, chemokines, signal transduction proteins, housekeeping proteins, cell cycle regulation proteins, transcription factors, zinc finger proteins, chromatin remodeling proteins, etc. Once a classifier gene or set of classifier genes is analyzed in a particular biological sample, the results are compared to the reference sets of the Tables 1-6. The physiological state of the sample can then be determined. Information gained from the analysis of classifier genes in a sample can be used in to diagnose the potential for the disease to progress, the actual stage to which a disease has progressed (e.g. metastatic colorectal cancer), or to monitor the efficacy of therapeutic regimens given to a patient.

RNA or protein can be isolated and assayed from a biological sample using any techniques, for example, they can be isolated from fresh or frozen biopsy, from formalin-fixed tissue, from body fluids, such as blood, plasma, serum, urine, or sputum. Of course the present invention is not limited to the nature of the samples or the nature of the comparison, and will find use in a variety of applications.

The treatment of cancer has been hampered by the fact that there is considerable heterogeneity even within one type of cancer. Some cancers, for example, have the ability to invade tissues and display an aggressive course of growth characterized by metastases. These tumors generally are associated with a poor outcome for the patient. And yet, without a means of identifying such tumors and distinguishing such tumors from non-invasive cancer, the physician is at a loss to change and/or optimize therapy.

The present invention may be used to compare normal tissue with cancer tissue, as well as to differentiate between cancer tissue that is non-metastatic, cancer that is metastatic, and cancer tissue that has a potential to metastasize.

In yet another embodiment, the present invention may be used to determine the health status of a cell culture, tissue, or organ.

The present invention also finds use in drug screening. For example, samples treated with different candidate drugs can be subjected to the methods of the present invention to determine the ability of the compounds to alter the expression of classifier genes known to be implicated in the disease state. For example, if a particular classifier gene is known to be over-expressed in cancer cells, one can look for drugs that reduce the expression of the suspect gene or set of genes to normal levels.

Analysis of gene expression may be at the gene transcript or the protein level. The amount of gene expression may be evaluated using nucleic acid probes to the DNA or RNA equivalent of the gene transcript. Alternatively, the final gene product itself (protein) can be monitored, for example, with antibodies to the classifier protein and standard immunoassays (ELISAs, etc.) or other techniques, including mass spectroscopy assays, 2D gel electrophoresis assays, etc. Proteomics and separation techniques may also allow quantification of expression.

In a preferred embodiment, gene expression monitoring is performed simultaneously on a number of genes. Multiple protein expression monitoring can be performed as well.

In one embodiment, the classifier gene nucleic acid probes are attached to biochips as outlined herein for the detection and quantification of nucleotide sequences in a particular cell or tissue.

General Recombinant DNA Methods

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kD) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Cloning Methods for the Isolation of Nucleotide Sequences

In general, nucleic acid sequences are cloned from cDNA and genomic DNA libraries or isolated using amplification techniques such as polymerase chain reaction (PCR). The primers used for PCR may amplify either the full length sequence or a probe of one to several hundred nucleotides, which is subsequently used to screen a library for full-length clones. Various combinations of oligonucleotides can be used to amplify coding and non-coding regions of the nucleotide sequence.

Nucleic acids can also be isolated from expression libraries using antibodies as probes. Polyclonal or monoclonal antibodies can be raised using the translation of a coding sequence, or any immunogenic portion thereof.

To make a cDNA library, one should choose a source that is rich in mRNA of the molecule one desires to clone. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating specific nucleic acids and their orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of target molecules directly from mRNA, from cDNA, from genomic libraries or cDNA libraries. Degenerate oligonucleotides can be designed to amplify target molecules homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of target molecule-encoding mRNA in physiological samples, for nucleic acid sequencing, or for other purposes. Genes amplified by the PCR reaction can be purified from agarose gels and cloned into an appropriate vector.

Once isolated the nucleic acid is typically cloned into intermediate vectors before transformation into prokaryotic or eukaryotic cells for replication and/or expression. These intermediate vectors are typically prokaryote vectors, e.g., plasmids, or shuttle vectors.

Expression of Cloned Nucleotide Sequences in Prokaryotes and Eukaryotes

To obtain high level expression of a cloned gene, one typically subclones the gene into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al., supra. Bacterial expression systems for expressing the target proteins are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the target molecule-encoding nucleic acid in host cells. A typical expression cassette thus contains a promoter operably linked to the nucleic acid sequence encoding target molecules and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a target molecule-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical—any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of target protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the gene.

After the expression vector is introduced into the cells, the transfected cells are cultured under conditions favoring expression of the gene or gene fragment. The product of the expressed gene or gene fragment is then recovered from the culture using standard techniques identified below.

Purification of Classifier Gene Polypeptides

Either naturally occurring or recombinant proteins can be purified and used to generate antibodies. Naturally occurring proteins can be purified from a variety of sources. However, in a preferred embodiment the proteins are isolated from mammalian tissue. In a particularly preferred embodiment, the proteins are isolated from human tissue. Recombinant classifier proteins can be purified from any suitable expression system.

The proteins may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant proteins are being purified all are familiar to those of skill in the art. For example, proteins having established molecular adhesion properties can be reversibly fused to another protein. With the appropriate ligand, the protein of interest may be selectively adsorbed to a purification column and then freed from the column in a relatively pure form. The fused protein is then removed by enzymatic activity. Finally, if antibodies to a portion of the protein are available, the protein may be purified using immunoaffinity columns.

Antibodies to Classifier Gene Polypeptides

Where the classifier gene product is a polypeptide encoded by a polynucleotide of the Tables 1-6, gene expression profiling can be examined using antibodies to the expressed classifier proteins.

To make effective antibodies, the classifier protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller classifier protein will be able to bind to the full-length protein, particularly linear epitopes. In a preferred embodiment, the epitope is unique; that is, antibodies generated to a unique epitope show little or no cross-reactivity.

Both polyclonal and monoclonal antibodies may be raised against the classifier proteins encoded by the classifier genes shown in the reference sets of the Tables 1-6. Methods of producing polyclonal and monoclonal antibodies that react specifically with specific proteins are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors (see Winthrop et al., Q J Nucl Med 44:284-95 (2000)), as well as preparation of polyclonal and monoclonal antibodies by immunizing rabbits or mice (see, e.g., Huse et al., Science 246:1275-1281 (1989); Ward et al., Nature 341:544-546 (1989)). For some applications, recombinant antibody fragments derived from monoclonal antibodies—such as single-chain antibodies, diabodies, and minibodies—are preferred (see Wu and Yazaki, Q J Nucl Med 44:268-83 (2000)).

A number of immunogens comprising portions of classifier proteins encoded by the classifier genes of the Tables 1-6 may be used to produce antibodies specifically reactive with classifier proteins. For example, recombinant classifier proteins, or an antigenic fragment thereof can be isolated as is known in the art. Recombinant protein can be expressed in eukaryotic or prokaryotic cells, and then purified by well established methods known in the art. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the immunogen. When appropriately high titers of antibody to the immunogen are obtained, blood is collected from the animal, and antisera are prepared. Further fractionation of the antisera to enrich for antibodies reactive to the protein can be done if desired (see, Harlow & Lane, supra).

Monoclonal antibodies and polyclonal sera are collected and titered against the immunogen protein in an immunoassay, for example, a solid phase immunoassay with the immunogen immobilized on a solid support. Typically, polyclonal antisera with a titer of 104 or greater are selected and tested for their cross reactivity against non-homologous proteins and other family proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular protein ortholog can also be made, by subtracting out other cross-reacting orthologs from a species such as a non-human mammal.

Methods for Comparing Gene Expression Profiles with Reference Sets of the Tables 1-6

Patterns of gene expression can be compared to the reference set of the Tables 1-6 manually (by a person) or by a computer or other machine. An algorithm can be used to detect similarities and differences. The algorithm may score and compare, for example, the genes which are expressed and the genes which are not expressed. If the genes are expressed, the algorithm may further be used to quantify the expression by looking for relative changes in intensity of expression of a particular gene. A variety of algorithms for such comparisons are known in the art (see e.g. Breiman L, Friedman JH., Olshen RA, and Stone CJ. (1984) Classification and Regression Trees. Wadsworth and Brooks/Cole, Monterey Calif.)

Similarities in the gene expression profile of the classifier genes in a biological sample and a reference set may be determined with reference to which genes are expressed in both samples and/or which genes are not expressed in both samples. Alternatively, the relative differences in intensity of expression of two or more classifier genes in a sample, may be a basis for deciding similarity or difference. Differences in gene expression are considered significant when they are greater than 2-fold, 3-fold or 5-fold from the value defined by expression in a reference set of classifier genes.

Mathematical approaches can also be used to conclude whether similarities or differences in the gene expression exhibited by different samples are significant. See, e.g., Golub et al., Science 286, 531 (1999); Duda, et al. (2001) Pattern Classification Wiley; and Hastie, et al. (2001) The Elements of Statistical Learning: Data Mining, Inference, and Prediction Springer-Verlag. One approach to determine whether a sample is more similar to or has maximum similarity with a given condition between the sample and one or more pools representing different conditions for comparison; the pool with the smallest vector angle is then chosen as the most similar to the biological sample among the pools compared.

The gene expression patterns of the tissue sample will be compared against the expression patterns designated in the Tables 1-6. This comparison will lead to the determination of whether or not a sample has metastatic potential.

Differences in gene expression are considered significant when the differences in mean expressions across samples is detected with statistical significance and such that the level of falsely detected signficant genes is near zero (Efron B, Tibshirani R, Storey JD, and Tusher V. (2001) Empirical Bayes analysis of a microarray experiment. Journal of the American Statistical Association, 96: 1151-1160.)

Since the comparison of gene expression profiles can be made with computers or other machines as well as manually, the invention also provides for the storage and retrieval of a collection of data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 data records cross-tabulated with source.

The invention preferably provides a method for identifying peptide or nucleic acid sequences and determining the level of similarity or difference to a reference set, comprising performing a computerized comparison between a peptide or nucleic acid expression profiling record stored in or retrieved from a computer storage device or database and a reference set. The comparison can include a comparison algorithm or computer program embodiment thereof (e.g., FASTA, TFASTA, GAP, BESTFIT) and/or the comparison may be of the absolute or relative amount of a peptide or nucleic acid sequence in a pool of determined from a polypeptide or nucleic acid sample of a specimen.

The invention also provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting expression profiling data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an expression profiling result obtained by the method of the invention, and ranking database based on the degree of identity with one or more reference sets of the Tables 1-6. A central processor is preferably initialized to load and execute the computer program for comparison of the expression profiling results. Data for a query target is entered into the central processor via an I/O device. Execution of the computer program results in the central processor retrieving the expression profiling data from the data file, which comprises a binary description of an expression profiling result.

The expression profiling data and the computer program can be transferred to secondary memory, which is typically random access memory (e.g., DRAM, SRAM, SGRAM, or SDRAM). Expression profiles are ranked according to the degree of correspondence between an expression profile and one or more reference sets of the Tables 1-6. Results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also provides the use of a computer system, such as that described above, which comprises: (1) a computer; (2) a stored bit pattern encoding a collection of expression profiles obtained by the methods of the invention, which may be stored in the computer; (3) reference sets of the Tables 1-6, and (4) a program for comparison, typically with rank-ordering of comparison results on the basis of computed similarity values.

### EXAMPLES

### Example 1

### Identification of the Metastatic Potential of a Colorectal Cancer Tissue Sample Using Nucleic Acid and Antibody Based Assays

RNA can be extracted from tissue samples, and the presence or absence on metastatic colorectal cancer can be determined by comparing the expression profile of classifier genes in the sample to the defined sets of genes of the Tables 1-6. Analysis of the expression profile can be carried out by measuring expression levels of classifier gene mRNA or protein.

For example, tissue from a non-metastatic Duke's stage B primary tumor, and from colorectal cancer that has progressed to end stage liver metastasis. Expression profiles of classifier genes from each sample are generated by creating an expression profile of either nucleic acid based data, or protein based data. The information obtained in the expression profiling is then analyzed and compared so that the relative expression levels of classifier genes in the two samples is used to create reference sets of genes such as those provided in the Tables 1-6. Expression patterns from samples whose disease state is unknown can then be compared to the defined sets of classifier genes in the Tables 1-6 and the presence or absence of metastatic colorectal cancer is diagnosed. If metastatic colorectal cancer is diagnosed, then further analysis of the data can reveal the stage of the disease and the probable prognosis.

The analysis of mRNA is preferred. For mRNA analysis, labeled, e.g., fluorescent or biotinylated, RNA from the unknown sample may be analyzed with an oligonucleotide microarray comprising sequences corresponding to the classifier genes of the Tables 1-6. Techniques for analysis and set up of the microarrays are known in the art.

Results of the analysis are used to identify which classifier genes are expressed and the level of their expression (as judged by the intensity of the signal). The pattern generated by the microarray analysis is then compared to the defined sets of genes of the Tables 1-6, and a determination of whether metastatic colorectal cancer is present is made. If metastatic disease is present the stage of the disease can also be determined.

In another embodiment, an expression profile of a sample is generated by examining the protein expression pattern of the sample. In this embodiment, total protein is extracted from a sample of the tissue (e.g., liver). Total protein is run on an acrylamide gel, then analyzed by western blot using antibodies to classifier genes of the Tables 1-6. As in the case of mRNA analysis, the expression pattern revealed in the western blot is compared to the defined sets of genes of the Tables 1-6. A match between the expression pattern of the sample with a particular defined set or sets of genes of the Tables 1-6 will permit the determination of whether or not cancer is present.

The defined sets of classifier genes of the Tables 1-6 are superior in their predictive power, because their expression strongly correlates with colorectal cancer metastasis. These defined sets of genes therefore provide ready tools for the diagnosis and prognosis evaluation of cancer, particularly metastatic colorectal cancer.

### Example 2

### Protein Based Determination of Classifier gene Expression and Quantification of Expression Levels Using 2-Dimensional Gel Electrophoresis

The expression pattern of classifier genes can be determined from the expression pattern of the corresponding proteins. Classifier proteins can be identified, e.g., by their positions on a gel following 2-dimensional gel electrophoresis of a sample of tissue subject to analysis.

Methods of 2-dimensional gel electrophoresis are well known in the art. Well characterized proteins, such as the classifier genes of the Tables 1-6, can be isolated from their unique placement within a gel after separation according to, for example, isoelectric point in the first dimension and molecular size in the second dimension. Thus, it is possible to determine expression levels of classifier proteins in a sample, as well as absolute expression levels of classifier proteins without the need for preparation of classifier protein specific antibodies.

Expression profiles of classifier genes generated in this manner can by compared with the defined sets of genes of the Tables 1-6 and the metastatic potential of the sample can thereby be determined.

TABLE 1
Genes Differentially regulated in Metastatic Colorectal Cancer

| | Exemplar | | |
|---|---|---|---|
| Cluster | Accession | UniGene ID | UniGeneTitle |
| 1 | NA | Hs.76297 | G protein-coupled receptor kinase 6 (GPRK6), mRNA. |
| 1 | NM_173483 | NA | NM_173483 Homo sapiens hypothetical protein FLJ39501 (FLJ39501) |
| 1 | NM_003468.2 | NA | NM_003468.2\|Homo sapiens frizzled homolog 5 (Drosophila) (FZD5), mRNA |
| 1 | NA | NA | Target Exon |
| 1 | AC007050.25 | NA | ESTs |
| 1 | NA | NA | Target Exon |
| 1 | W25945 | Hs.8173 | hypothetical protein FLJ10803 |
| 1 | AW054922 | Hs.53478 | Homo sapiens cDNA FLJ12366 fis, clone MAMMA1002411 |
| 1 | AW847814 | Hs.289005 | Homo sapiens cDNA: FLJ21532 fis, clone COL06049 |
| 1 | BE244200 | Hs.406243 | KIAA0410 gene product |
| 1 | AW514668 | Hs.194258 | ESTs, Moderately similar to ALU5_HUMAN ALU SUBFAMILY SC SEQUENCE |
| | | | CONTAMINATION WARNING ENTRY [H. sapiens] |
| 1 | AA249096 | Hs.32793 | ESTs |
| 1 | L26953 | Hs.1010 | regulator of mitotic spindle assembly 1 |
| 1 | AI381687 | Hs.404198 | ESTs |
| 1 | N99638 | Hs.87409 | gb: za39g11.r1 Soares fetal liver spleen 1NFLS Homo sapiens cDNA clone 5′similar to |
| | | | contains Alu repetitive element;, mRNA sequence |
| 1 | AI205785 | Hs.190153 | ESTs |
| 1 | AW965212 | Hs.278871 | hypothetical protein FLJ30921 (FLJ30921), mRNA. |
| 1 | AL119442 | Hs.380968 | eukaryotic translation initiation factor 4 gamma, 2 |
| 1 | AA358045 | NA | gb: EST66944 Fetal lung III Homo sapiens cDNA 5′end similar to EST containing Alu |
| | | | repeat, mRNA sequence |
| 1 | AL050276 | Hs.159456 | zinc finger protein 288 |
| 1 | AI052358 | Hs.131741 | ESTs |
| 1 | AW976570 | Hs.97387 | ESTs |
| 1 | AI936504 | Hs.2083 | CDC-like kinase 1 |
| 1 | AA400079 | Hs.257854 | ESTs |
| 1 | AW883367 | Hs.356546 | hypothetical protein MGC5306 |
| 1 | AA417696 | Hs.372121 | ESTs |
| 1 | AA470152 | Hs.368209 | ESTs |
| 1 | AW971375 | Hs.292921 | ESTs |
| 1 | AW971070 | Hs.291160 | ESTs, Weakly similar to ALU1_HUMAN ALU SUBFAMILY J SEQUENCE |
| | | | CONTAMINATION WARNING ENTRY [H. sapiens] |
| 1 | T87431 | Hs.190738 | ESTs |
| 1 | AA531129 | Hs.190297 | ESTs |
| 1 | AW439330 | Hs.256889 | ESTs, Weakly similar to 2109260A B cell growth factor [H. sapiens] |
| 1 | AW157424 | Hs.280685 | ESTs, Weakly similar to I38022 hypothetical protein [H. sapiens] |
| 1 | AB040966 | Hs.83575 | KIAA1533 protein |
| 1 | AW188370 | Hs.250383 | Homo sapiens cDNA FLJ14279 fis, clone PLACE1005574 |
| 1 | AA628539 | Hs.57783 | Homo sapiens eukaryotic translation initiation factor 3, subunit 9 eta, 116 kDa (EIF3S9) |
| 1 | AA640770 | Hs.200994 | EST |
| 1 | AA664078 | NA | gb: ac04a05.s1 Stratagene lung (937210) Homo sapiens cDNA clone 3′similar to contains |
| | | | Alu repetitive element;, mRNA sequence |
| 1 | AA886511 | Hs.189282 | Homo sapiens cDNA: FLJ21429 fis, clone COL04205 |
| 1 | AA830893 | Hs.119769 | ESTs |
| 1 | BE327477 | Hs.166941 | ESTs |
| 1 | AI821940 | Hs.72071 | hypothetical protein FLJ20038 |
| 1 | AL137723 | Hs.5855 | Homo sapiens mRNA; cDNA DKFZp434D0818 (from clone DKFZp434D0818) |
| 1 | AA769874 | Hs.155287 | ubiquitin-protein isopeptide ligase (E3) |
| 1 | AI126162 | Hs.129037 | ESTs |
| 1 | AW748336 | Hs.168052 | KIAA0421 protein |
| 1 | AW083789 | Hs.124620 | ESTs |
| 1 | AI034357 | Hs.211194 | ESTs, Weakly similar to ALU8_HUMAN ALU SUBFAMILY SX SEQUENCE |
| | | | CONTAMINATION WARNING ENTRY [H. sapiens] |
| 1 | AW827419 | Hs.144139 | ESTs |
| 1 | BE262656 | Hs.32603 | hypothetical protein MGC3279 similar to collectins |
| 1 | AW469180 | Hs.346398 | ESTs |
| 1 | AI492857 | NA | gb: th72h08.x1 Soares_NhHMPu_S1 Homo sapiens cDNA clone 3′, mRNA sequence |
| 1 | AW451347 | Hs.175862 | ESTs |
| 1 | AI698091 | Hs.107845 | ESTs |
| 1 | AJ010046 | Hs.25155 | neuroepithelial cell transforming gene 1 |
| 1 | AL043983 | Hs.125063 | Homo sapiens cDNA FLJ13825 fis, clone THYRO1000558 |
| 1 | AW382884 | Hs.5320 | ESTs |
| 1 | BE378541 | Hs.279815 | cysteine sulfinic acid decarboxylase-relatedprotein 2 |
| 1 | R66282 | Hs.20247 | ESTs, Weakly similar to S65657 alpha-1C-adrenergic receptor splice form 2 [H. sapiens] |
| 1 | BE086548 | Hs.42346 | calcineurin-binding protein calsarcin-1 |
| 1 | AA907305 | Hs.36475 | ESTs |
| 2 | AF083130 | Hs.381498 | Homo sapiens CATX-14 mRNA, partial cds |
| 2 | NM_032446.1 | NA | NM_032446.1\|Homo sapiens (MEGF10), mRNA |
| 2 | NA | NA | Target Exon |
| 2 | AW152207 | Hs.270977 | ESTs, Weakly similar to I38022 hypothetical protein [H. sapiens] |
| 2 | AA601038 | Hs.191797 | ESTs, Weakly similar to S65657 alpha-1C-adrenergic receptor splice form 2 [H. sapiens] |
| 2 | U28831 | Hs.44566 | KIAA1641 protein |
| 2 | AV660717 | Hs.47144 | DKFZP586N0819 protein |
| 2 | AW444816 | Hs.171537 | hypothetical protein FLJ21596 |
| 2 | AW589558 | Hs.299883 | hypothetical protein FLJ23399 |
| 2 | AW590680 | Hs.355571 | Von Willebrand factor |
| 2 | AW770280 | Hs.36258 | ESTs, Moderately similar to JC5238 galactosylceramide-like protein, GCP [H. sapiens] |
| 2 | AW451618 | Hs.380683 | ESTs |
| 2 | BE242691 | Hs.14947 | ESTs |
| 2 | AI056689 | Hs.133538 | ESTs, Weakly similar to ALU1_HUMAN ALU SUBFAMILY J SEQUENCE |
| | | | CONTAMINATION WARNING ENTRY [H. sapiens] |
| 2 | BE081585 | NA | gb: QV2-BT0635-210400-156-b07 BT0635 Homo sapiens cDNA, mRNA sequence |
| 2 | AI056885 | Hs.133539 | ESTs |
| 2 | BE336632 | Hs.278850 | hypothetical protein FLJ13687 |
| 2 | AA827082 | Hs.291872 | ESTs |
| 2 | R11661 | Hs.14165 | ESTs, Moderately similar to ALU5_HUMAN ALU SUBFAMILY SC SEQUENCE |
| | | | CONTAMINATION WARNING ENTRY [H. sapiens] |
| 2 | R39769 | Hs.379238 | ESTs, Moderately similar to ALU8_HUMAN ALU SUBFAMILY SX SEQUENCE |
| | | | CONTAMINATION WARNING ENTRY [H. sapiens] |
| 2 | AA188645 | Hs.250638 | Homo sapiens mRNA full length insert cDNA clone EUROIMAGE 152428 |
| 2 | C75563 | Hs.113029 | ribosomal protien S25 |
| 2 | U90916 | Hs.82845 | Homo sapiens cDNA: FLJ21930 fis, clone HEP04301, highly similar to HSU90916 |
| | | | Human clone 23815 mRNA sequence |
| 2 | AA601036 | Hs.285083 | ESTs |
| 2 | BE271922 | Hs.406392 | ESTs, Weakly similar to zinc finger protein [H. sapiens] |
| 2 | AA830402 | Hs.221216 | ESTs |
| 2 | AW975051 | Hs.192044 | ESTs, Weakly similar to I78885 serine/threonine-specific protein kinase [H. sapiens] |
| 2 | AL080172 | Hs.105894 | hypothetical protein FLJ21919 |
| 2 | AA310919 | Hs.7369 | Homo sapiens cDNA FLJ14343 fis, clone THYRO1000916 |
| 2 | AI457640 | Hs.206632 | ESTs |
| 2 | AA335715 | Hs.98132 | ESTs |
| 2 | T94907 | Hs.188572 | ESTs |
| 2 | AI174861 | Hs.190623 | ESTs |
| 2 | AW881411 | Hs.169078 | hypothetical protein FLJ23018 |
| 2 | AA554827 | Hs.370705 | DKFZp434A0131 protein |
| 2 | H72531 | Hs.36190 | ESTs |
| 2 | AL042436 | Hs.97723 | ESTs |
| 2 | AI656478 | Hs.321622 | hypothetical protein FLJ20363 |
| 2 | AA417614 | Hs.136825 | ESTs |
| 2 | AI016712 | Hs.2877971 | integrin, beta 1 (fibronectin receptor, beta polypeptide, antigen CD29 includes MDF2, |
| | | | MSK12) |
| 2 | AA769365 | Hs.126058 | ESTs |
| 2 | AA464964 | NA | gb: zx80f10.s1 Soares ovary tumor NbHOT Homo sapiens cDNA clone 3′, mRNA |
| | | | sequence |
| 2 | AA847744 | Hs.370675 | ESTs |
| 2 | AW079559 | Hs.152258 | ESTs |
| 2 | AI417881 | Hs.292464 | ESTs |
| 2 | BE350122 | Hs.157367 | ESTs, Weakly similar to 178885 serine/threonine-specific protein kinase [H. sapiens] |
| 2 | AA503053 | Hs.81474 | ESTs |
| 2 | AA699965 | Hs.369440 | ESTs |
| 2 | AI660840 | Hs.191202 | ESTs, Weakly similar to ALUE_HUMAN !!!! ALU CLASS E WARNING ENTRY !!! |
| | | | [H. sapiens] |
| 2 | AI341227 | Hs.157106 | ESTs |
| 2 | AA830532 | Hs.372176 | ESTs |
| 2 | BE217838 | Hs.152492 | ESTs |
| 2 | AA878324 | NA | ESTs |
| 2 | AW362945 | Hs.162459 | ESTs |
| 2 | AW296280 | Hs.152016 | Homo sapiens cDNA: FLJ22140 fis, clone HEP20977 |
| 2 | AI241331 | Hs.75113 | general transcription factor IIIA |
| 2 | AF039697 | Hs.132883 | serologically defined colon cancer antigen 31 |
| 2 | AW390125 | Hs.240443 | Homo sapiens cDNA: FLJ23538 fis, clone LNG08010, highly similar to BETA2 Human |
| | | | MEN1 region clone epsilon/beta mRNA |
| 2 | AI208611 | Hs.333555 | Homo sapiens cDNA FLJ11720 fis, clone HEMBA1005293 |
| 2 | AA610649 | Hs.333239 | ESTs |
| 2 | AF119913 | Hs.404158 | Homo sapiens PRO3077 mRNA, complete cds |
| 2 | AF132730 | Hs.149784 | hypothetical protein |
| 2 | AW974949 | Hs.87409 | ESTs |
| 2 | AI654144 | Hs.271511 | ESTs, Weakly similar to I78885 serine/threonine-specific protein kinase [H. sapiens] |
| 2 | R26877 | Hs.24128 | ESTs |
| 2 | BE551618 | Hs.82285 | phosphoribosylglycinamide formyltransferase, phosphoribosylglycinamide synthetase, |
| | | | phosphoribosylaminoimidazole synthetase |
| 2 | AA744692 | Hs.166539 | ESTs |
| 2 | AL038624 | Hs.208752 | ESTs, Weakly similar to ALU8_HUMAN ALU SUBFAMILY SX SEQUENCE |
| | | | CONTAMINATION WARNING ENTRY [H. sapiens] |
| 2 | AL080280 | Hs.383970 | gb: Homo sapiens mRNA full length insert cDNA clone EUROIMAGE 85905 |
| 2 | AA766142 | Hs.131810 | hypothetical protein FLJ35976 (FLJ35976), mRNA. |
| 2 | BE466173 | Hs.145696 | splicing factor (CC1.3) |
| 2 | W78940 | Hs.20526 | ESTs |
| 2 | AI767388 | Hs.37890 | Human DNA sequence from clone RP5-1024N4 on chromosome 1p32.1-33. Contains the |
| | | | gene for a novel Sodium: solute symporter family member similar to SLC5A1 (SGLT1), a |
| | | | pseudogene similar to part of butyrophilin family members, a novel gene, ESTs, STSs, GS |
| 2 | R71264 | Hs.16798 | ESTs |
| 2 | BE550891 | Hs.270624 | ESTs |
| 2 | NM_014135 | Hs.8345 | PRO0641 protein |
| 2 | AI076570 | Hs.134053 | ESTs |
| 2 | AI371823 | Hs.34079 | ESTs |
| 2 | AF169312 | Hs.9613 | PPAR(gamma) angiopoietin related protein |
| 2 | AI344782 | Hs.349261 | DnaJ (Hsp40) homolog, subfamily C, member 3 |
| 2 | AI174603 | Hs.254105 | enolase 1, (alpha) |
| 2 | AL040482 | Hs.286173 | KIAA1595 protein |
| 2 | AI670843 | Hs.370292 | ESTs |
| 2 | AI022813 | Hs.92679 | Homo sapiens clone CDABP0014 mRNA sequence |
| 2 | AF113925 | Hs.19405 | caspase recruitment domain 4 |
| 2 | H65629 | Hs.245997 | ESTs |
| 2 | T62926 | Hs.304184 | ESTs |
| 2 | AA353125 | Hs.184721 | ESTs |
| 2 | N33622 | NA | gb: yv22h10.s1 Soares fetal liver spleen 1NFLS Homo sapiens cDNA elone 3′, mRNA |
| | | | sequence |
| 2 | AA002207 | Hs.17385 | Homo sapiens clone IMAGE: 119716, mRNA sequence |
| 2 | AB020714 | Hs.24656 | KIAA0907 protein |
| 2 | AI218945 | Hs.226925 | ESTs |
| 2 | AA847992 | Hs.137003 | ESTs |
| 2 | AI924046 | Hs.119567 | ESTs, Weakly similar to A47582 B-cell growth factor precursor [H. sapiens] |
| 2 | AL040914 | NA | gb: DKFZp434J2015_s1 434 (synonym: htes3) Homo sapiens cDNA clone |
| | | | DKFZp434J2015 3′, mRNA sequence |
| 2 | AA683416 | Hs.209061 | sudD suppressor of bimD6 homolog (A. nidulans) (SUDD), transcript variant 1, mRNA. |
| 2 | AW058464 | Hs.386465 | protein with polyglutamine repeat; calcium (ca2) homeostasis endoplasmic reticulum |
| | | | protein |
| 2 | BE549380 | Hs.307034 | Homo sapiens, clone IMAGE: 3460539, mRNA, partial cds |
| 3 | U49973 | NA | gb: Human Tigger1 transposable element, complete consensus sequence. |
| 3 | AI689496 | Hs.108932 | ESTs |
| 3 | AW293452 | Hs.16228 | ESTs |
| 3 | AA776721 | Hs.85603 | down-regulated by Ctnnb1, a |
| 3 | AA581602 | Hs.41840 | ESTs |
| 3 | AI801098 | Hs.151500 | ESTs |
| 3 | AA740616 | NA | gb: ny97f11.s1 NCI_CGAP_GCB1 Homo sapiens cDNA clone 3′, mRNA sequence |
| 3 | AI807519 | Hs.104520 | Homo sapiens cDNA FLJ13694 fis, clone PLACE2000115 |
| 3 | AA327092 | NA | ESTs |
| 3 | AA602917 | Hs.325520 | LAT1-3TM protein |
| 3 | NM_005781 | Hs.153937 | activated p21cdc42Hs kinase |
| 3 | AA640987 | Hs.193767 | ESTs |
| 3 | AA135370 | Hs.188536 | Homo sapiens cDNA: FLJ21635 fis, clone COL08233, highly similar to AF131819 Homo |
| | | | sapiens clone 24838 mRNA sequence |
| 3 | AW296451 | Hs.24605 | ESTs |
| 3 | AW299534 | Hs.105739 | ESTs |
| 3 | U26710 | Hs.3144 | Cas-Br-M (murine) ectropic retroviral transforming sequence b |
| 3 | AW362803 | Hs.166271 | ESTs |
| 3 | AW975895 | NA | ESTs |
| 3 | AW450376 | Hs.378828 | KIAA0665 gene product |
| 3 | AI002106 | Hs.15670 | ESTs |
| 3 | AA811347 | NA | gb: ob81h06.s1 NCI_CGAP_GCBI Homo sapiens cDNA clone 3′, mRNA sequence |
| 3 | AI798851 | Hs.356716 | hemoglobin, gamma G |
| 3 | F06700 | Hs.7879 | interferon-related developmental regulator 1 |
| 3 | AI564835 | Hs.381225 | ESTs, Weakly similar to Z195_HUMAN ZINC FINGER PROTEIN 195 [H. sapiens] |
| 3 | AW016607 | Hs.201582 | ESTs |
| 3 | AB007928 | Hs.374987 | KIAA0459 protein |
| 3 | S72043 | Hs.73133 | metallothionein 3 (growth inhibitory factor (neurotrophic)) |
| 3 | AA228357 | Hs.399939 | gb: nc39d05.r1 NCI_CGAP_Pr2 Homo sapiens cDNA clone, mRNA sequence |
| 4 | AA130986 | Hs.271627 | ESTs |
| 4 | T64896 | Hs.406798 | Homo sapiens cDNA FLJ11533 fis, clone HEMBA1002678 |
| 4 | AA132637 | Hs.15396 | Homo sapiens, clone IMAGE: 3948909, mRNA, partial cds |
| 4 | AA317962 | Hs.249721 | ESTs, Moderately similar to PC4259 ferritin associated protein [H. sapiens] |
| 4 | AW167439 | Hs.190651 | Homo sapiens cDNA FLJ13625 fis, clone PLACE1011032 |
| 4 | AW452823 | Hs.135268 | ESTs |
| 4 | AA132255 | Hs.143951 | ESTs |
| 4 | D83782 | Hs.78442 | SREBP CLEAVAGE-ACTIVATING PROTEIN |
| 4 | AI690465 | Hs.201661 | ESTs, Weakly similar to JC5238 galactosylceramide-like protein, GCP [H. sapiens] |
| 4 | R07785 | Hs.429867 | ESTs |
| 4 | AL041465 | Hs.182982 | golgin-67 |
| 4 | AW183695 | Hs.370907 | ESTs |
| 4 | AW276914 | Hs.423341 | Homo sapiens clone IMAGE: 713177, mRNA sequence |
| 4 | U50535 | Hs.110630 | Human BRCA2 region, mRNA sequence CG006 |
| 4 | AF073931 | Hs.122359 | calcium channel, voltage-dependent, alpha 1 H subunit |
| 4 | AW341131 | Hs.146345 | ESTs |
| 4 | BE176694 | Hs.279860 | tumor protein, translationally-controlled 1 |
| 4 | AW963118 | Hs.161784 | ESTs |
| 4 | AW513691 | Hs.270149 | ESTs, Weakly similar to 2109260A B cell growth factor [H. sapiens] |
| 4 | BE173380 | Hs.381903 | ESTs |
| 4 | Z29067 | Hs.2236 | NIMA (never in mitosis gene a)-related kinase 3 |
| 4 | AA425310 | Hs.155766 | ESTs, Weakly similar to A47582 B-cell growth factor precursor [H. sapiens] |
| 4 | AW973253 | Hs.292689 | ESTs |
| 4 | AA453987 | Hs.144802 | ESTs |
| 4 | AA612710 | Hs.284148 | ESTs |
| 4 | AA830335 | Hs.105273 | ESTs |
| 4 | AW970859 | Hs.313503 | ESTs |
| 4 | AA532718 | Hs.178604 | ESTs |
| 4 | AI459519 | Hs.314437 | clone IMAGE: 4607209, mRNA sequence [H. sapiens] |
| 4 | BE263901 | Hs.381222 | ESTs, Weakly similar to S37431 ankyrin 2, neuronal long splice form [H. sapiens] |
| 4 | AI301080 | Hs.35276 | KIAA0852 protein |
| 4 | AW975009 | Hs.292274 | ESTs, Weakly similar to A46010 X-linked retinopathy protein [H. sapiens] |
| 4 | AA677540 | Hs.117064 | ESTs |
| 4 | H74319 | Hs.188620 | ESTs |
| 4 | AI800041 | Hs.369733 | ESTs |
| 4 | AL360140 | Hs.176005 | Homo sapiens mRNA full length insert cDNA clone EUROIMAGE 113222 |
| 4 | AF134160 | Hs.7327 | claudin 1 |
| 4 | AI982794 | Hs.159473 | ESTs |
| 4 | AK001631 | Hs.8083 | hypothetical protein FLJ10769 |
| 4 | W22152 | Hs.282929 | ESTs |
| 4 | H77824 | NA | ESTs |
| 4 | AU076643 | Hs.313 | secreted phosphoprotein 1 (osteopontin, bone sialoprotein I, early T-lymphocyte activation |
| | | | 1) |
| 4 | AW958124 | Hs.142442 | HP1-BP74 |
| 4 | AL137714 | Hs.356298 | hypothetical protein LOC58481 |
| 4 | AA001266 | Hs.133521 | ESTs |
| 4 | AL133100 | Hs.377705 | hypothetical protein FLJ20531 |
| 4 | AA001615 | Hs.84561 | ESTs |
| 4 | AA568515 | Hs.293510 | ESTs |
| 4 | AW079749 | Hs.184719 | ESTs, Weakly similar to ALU1_HUMAN ALU SUBFAMILY J SEQUENCE |
| | | | CONTAMINATION WARNING ENTRY [H. sapiens] |
| 4 | AL045285 | Hs.277401 | bromodomain adjacent to zinc finger domain, 2A |
| 4 | AI740647 | Hs.141012 | ESTs, Weakly similar to ALU1_HUMAN ALU SUBFAMILY J SEQUENCE |
| | | | CONTAMINATION WARNING ENTRY [H. sapiens] |
| 4 | AW976347 | Hs.76966 | ESTs |
| 4 | AI191811 | Hs.54629 | ESTs |
| 5 | NA | NA | Target Exon |
| 5 | NA | NA | Target Exon |
| 5 | NA | NA | C7002129*: gi\|3638957\|gb\|AAC36301.1\|(AC004877) sco-spondin-mucin-like; similar to |
| | | | P98167 ( |
| 5 | AW883529 | Hs.173830 | ESTs, Weakly similar to ALU7_HUMAN ALU SUBFAMILY SQ SEQUENCE |
| | | | CONTAMINATION WARNING ENTRY [H. sapiens] |
| 5 | AW969543 | Hs.144609 | mitogen-activated protein kinase kinase kinase 13 |
| 5 | AW854536 | NA | gb: RC3-CT0255-200100-024-a08 CT0255 Homo sapiens cDNA, mRNA sequence |
| 5 | AA156657 | Hs.332383 | ESTs |
| 5 | N65993 | Hs.294003 | ESTs, Weakly similar to ALU1_HUMAN ALU SUBFAMILY J SEQUENCE |
| | | | CONTAMINATION WARNING ENTRY [H. sapiens] |
| 5 | BE275835 | NA | gb: 601121639F1 NIH_MGC_20 Homo sapiens cDNA clone 5′, mRNA sequence |
| 5 | H02480 | Hs.79592 | ESTs |
| 5 | AL038450 | Hs.48948 | ESTs |
| 5 | AA177088 | Hs.190065 | ESTs |
| 5 | AA203569 | Hs.191482 | ESTs |
| 5 | AI253112 | Hs.133540 | ESTs |
| 5 | T85105 | NA | ESTs |
| 5 | AI972919 | Hs.118837 | obscurin, cytoskeletal calmodulin and titin-interacting RhoGEF |
| 5 | AA304999 | Hs.27301 | ESTs, Weakly similar to similar to KIAA0855 [H. sapiens] |
| 5 | AA284447 | Hs.271887 | ESTs |
| 5 | AF182277 | Hs.330780 | cytochrome P450, subfamily IIB (phenobarbital-inducible), polypeptide 7 |
| 5 | AI760018 | Hs.205071 | ESTs |
| 5 | R66740 | Hs.110613 | KIAA0220 protein |
| 5 | BE296394 | NA | gb: 601176734F1 NIH_MGC_17 Homo sapiens cDNA clone 5′, mRNA sequence |
| 5 | AW960454 | NA | ESTs |
| 5 | H57111 | Hs.221132 | ESTs |
| 5 | R42755 | Hs.23096 | ESTs |
| 5 | AA367069 | Hs.100636 | ESTs |
| 5 | AL049987 | Hs.166361 | Homo sapiens mRNA; cDNA DKFZp564F112 (from clone DKFZp564F112) |
| 5 | AI767152 | Hs.181400 | ESTs, Weakly similar to 178885 serine/threonine-specific protein kinase [H. sapiens] |
| 5 | AW971063 | Hs.292882 | ESTs |
| 5 | AI494291 | Hs.369171 | ESTs |
| 5 | AI734110 | Hs.136355 | ESTs |
| 5 | AI123657 | Hs.169755 | ESTs, Weakly similar to JC5314 CDC28/cdc2-like kinase associating arginine-serine |
| | | | cyclophilin [H. sapiens] |
| 5 | AA488953 | NA | gb: aa55e05.r1 NCI_CGAP_GCB1 Homo sapiens cDNA clone 5′, mRNA sequence |
| 5 | AW295859 | Hs.235860 | ESTs |
| 5 | AA806538 | Hs.130732 | KIAA1575 protein |
| 5 | AL040360 | Hs.162203 | ESTs, Weakly similar to alternatively spliced product using exon 13A [H. sapiens] |
| 5 | N38913 | Hs.221575 | ESTs |
| 5 | AW971983 | Hs.293003 | cation channel, sperm associated 2 (CATSPER2), transcript variant 1, mRNA. |
| 5 | AI343966 | Hs.158528 | ESTs |
| 5 | AW136134 | Hs.220277 | ESTs |
| 5 | AW450922 | Hs.112478 | ESTs |
| 5 | AA609738 | Hs.16525 | ESTs |
| 5 | AA613792 | NA | gb: no97h03.s1 NCI_CGAP_Pr2_Homo sapiens cDNA clone, mRNA sequence |
| 5 | AI631749 | Hs.156616 | ESTs, Weakly similar to alternatively spliced product using exon 13A [H. sapiens] |
| 5 | H56995 | Hs.37372 | Homo sapiens DNA binding peptide mRNA, partial cds |
| 5 | AI624436 | Hs.310286 | ESTs |
| 5 | AW374941 | Hs.87409 | ESTs |
| 5 | AW974957 | Hs.288719 | Homo sapiens cDNA FLJ12142 fis, clone MAMMA1000356 |
| 5 | AA737345 | Hs.294041 | ESTs |
| 5 | AA888311 | Hs.17602 | Homo sapiens cDNA FLJ12381 fis, clone MAMMA1002566 |
| 5 | AW295687 | Hs.254420 | ESTs |
| 5 | AA757900 | Hs.270823 | ESTs, Weakly similar to S65657 alpha-1C-adrenergic receptor splice form 2 [H. sapiens] |
| 5 | AI916685 | Hs.371850 | ESTs |
| 5 | BE273296 | Hs.3069 | Homo sapiens cDNA FLJ13255 fis, clone OVARC1000800, moderately similar to |
| | | | MITOCHONDRIAL STRESS-70 PROTEIN PRECURSOR |
| 5 | AA808948 | Hs.378776 | ESTs, Moderately similar to ALU1_HUMAN ALU SUBFAMILY J SEQUENCE |
| | | | CONTAMINATION WARNING ENTRY [H. sapiens] |
| 5 | BE046594 | NA | gb: hn41c11.x1 NCI_CGAP_RDF2 Homo sapiens cDNA clone 3′, mRNA sequence |
| 5 | AI277986 | Hs.164875 | ESTs |
| 5 | AA830144 | Hs.135613 | ESTs, Moderately similar to I38022 hypothetical protein [H. sapiens] |
| 5 | BE159253 | Hs.300638 | ESTs |
| 5 | BE561880 | NA | gb: 601346073F1 NIH_MGC_8 Homo sapiens cDNA clone 5′, mRNA sequence |
| 5 | AI565071 | Hs.369984 | ESTs |
| 5 | AI184717 | Hs.372653 | ESTs |
| 5 | AI052572 | NA | ESTs, Weakly similar to ALU1_HUMAN ALU SUBFAMILY J SEQUENCE |
| | | | CONTAMINATION WARNING ENTRY [H. sapiens] |
| 5 | AI056776 | Hs.133397 | ESTs, Weakly similar to I78885 serine/threonine-specific protein kinase [H. sapiens] |
| 5 | AI123195 | Hs.47783 | gb: oo17a10.x1 Soares_NSF_F8_9W_OT_PA_P_S1 Homo sapiens cDNA clone 3′ similar |
| | | | to TR: Q16673 Q16673 PMS7 MRNA; contains OFR.t1 OFR repetitive element;, mRNA |
| | | | sequence |
| 5 | AI565004 | Hs.374415 | cathepsin D (lysosomal aspartyl protease) |
| 5 | AI858635 | Hs.144763 | ESTs |
| 5 | AL049951 | Hs.22370 | Homo sapiens mRNA; cDNA DKFZp564O0122 (from clone DKFZp564O0122) |
| 5 | AI880843 | Hs.370296 | ESTs |
| 5 | AI653006 | Hs.195374 | ESTs |
| 5 | AI990790 | Hs.188614 | ESTs |
| 5 | AA004681 | Hs.59432 | ESTs |
| 5 | AA004906 | Hs.404424 | ESTs |
| 5 | AI826999 | Hs.224624 | ESTs |
| 5 | AA737314 | Hs.194324 | hypothetical protein FLJ12634 |
| 5 | AA011616 | NA | ESTs |
| 5 | AW504178 | Hs.222731 | ESTs, Weakly similar to I38022 hypothetical protein [H. sapiens] |
| 5 | AB032995 | Hs.26440 | two-pore channel 1, homolog |
| 5 | AA454220 | Hs.61170 | ESTs |
| 5 | AI914925 | Hs.222240 | ESTs |
| 5 | BE066058 | Hs.269233 | ESTs, Moderately similar to I78885 serine/threonine-specific protein kinase [H. sapiens] |
| 5 | H62793 | Hs.268945 | ESTs |
| 5 | AW295097 | Hs.200260 | ESTs |
| 6 | AA075144 | Hs.401448 | gb: zm86f06.s1 Stratagene ovarian cancer (937219) Homo sapiens cDNA clone |
| | | | IMAGE: 544835 3′ similar to gb: X16064 TRANSLATIONALLY CONTROLLED |
| | | | TUMOR PROTEIN (HUMAN);, mRNA sequence. |
| 6 | AI539227 | Hs.214039 | hypothetical protein FLJ23556 |
| 6 | AA031576 | Hs.143812 | Homo sapiens cDNA FLJ12956 fis, clone NT2RP2005501 |
| 6 | AF045458 | Hs.47061 | unc-51 (C. elegans)-like kinase 1 |
| 6 | AW631439 | NA | Homo sapiens cDNA FLJ11582 fis, clone HEMBA1003656 |
| 6 | NM_014760 | Hs.75863 | KIAA0218 gene product |
| 6 | C14904 | Hs.45184 | Homo sapiens cDNA FLJ12284 fis, clone MAMMA1001757 |
| 6 | AA148984 | Hs.48849 | ESTs, Weakly similar to ALU4_HUMAN ALU SUBFAMILY SB2 SEQUENCE |
| | | | CONTAMINATION WARNING ENTRY [H. sapiens] |
| 6 | AW602463 | Hs.233370 | ESTs |
| 6 | X78342 | Hs.77313 | cyclin-dependent kinase (CDC2-like) 10 |
| 6 | R12228 | NA | ESTs |
| 6 | T61572 | Hs.79385 | Human clone 23574 mRNA sequence |
| 6 | AB020671 | Hs.84883 | KIAA0864 protein |
| 6 | AA236282 | Hs.172318 | ESTs |
| 6 | AA323486 | Hs.325530 | Homo sapiens cDNA FLJ12335 fis, clone MAMMA1002219, highly similar to Rattus |
| | | | norvegicus rexo70 mRNA |
| 6 | BE247348 | Hs.155499 | golgi-specific brefeldin A resistance factor 1 |
| 6 | R05327 | Hs.189726 | ESTs |
| 6 | T19228 | Hs.172572 | hypothetical protein FLJ20093 |
| 6 | AW979298 | Hs.292896 | ESTs |
| 6 | AW812795 | Hs.337534 | ESTs, Moderately similar to I38022 hypothetical protein [H. sapiens] |
| 6 | AA489166 | Hs.156933 | ESTs |
| 6 | BE218886 | Hs.282070 | ESTs |
| 6 | AF043244 | Hs.278439 | nucleolar protein 3 (apoptosis repressor with CARD domain) |
| 6 | AI076345 | Hs.373742 | ESTs |
| 6 | BE552155 | Hs.294035 | ESTs, Weakly similar to ALU5_HUMAN ALU SUBFAMILY SC SEQUENCE |
| | | | CONTAMINATION WARNING ENTRY [H. sapiens] |
| 6 | AW847208 | Hs.406201 | BANP homolog, SMAR1 homolog |
| 6 | AA834082 | Hs.307559 | ESTs |
| 6 | AF119847 | Hs.383393 | Homo sapiens PRO1550 mRNA, partial cds |
| 6 | AW352170 | Hs.129086 | Homo sapiens cDNA FLJ12007 fis, clone HEMBB1001588 |
| 6 | AI189587 | Hs.120915 | ESTs |
| 6 | AA677934 | Hs.117864 | ESTs |
| 6 | AA700946 | Hs.368238 | ESTs |
| 6 | AI684710 | Hs.111611 | ribosomal protein L27 |
| 6 | AW022213 | Hs.370487 | ESTs |
| 6 | AA580691 | Hs.180789 | S164 protein |
| 6 | AW975663 | Hs.293404 | ESTs, Weakly similar to ALU1_HUMAN ALU SUBFAMILY J SEQUENCE |
| | | | CONTAMINATION WARNING ENTRY [H. sapiens] |
| 6 | AW369770 | Hs.130351 | ESTs |
| 6 | AI380429 | Hs.172445 | ESTs |
| 6 | AA356599 | Hs.173904 | ESTs |
| 6 | BE560954 | NA | gb: 601347719F1 NIH_MGC_8 Homo sapiens cDNA clone 5′, mRNA sequence |
| 6 | AL040215 | Hs.7278 | cryptochrome 2 (photolyase-like) |
| 6 | AI376551 | Hs.368882 | gb: te64e10.x1 Soares_NFL_T_GBC_S1 Homo sapiens cDNA clone 3′, mRNA sequence |
| 6 | AI247472 | Hs.132965 | ESTs |
| 6 | AL038823 | Hs.12840 | Homo sapiens germline mRNA sequence |
| 6 | AW450103 | Hs.151124 | ESTs |
| 6 | AK001579 | Hs.25277 | hypothetical protein FLJ21065 |
| 6 | W80462 | NA | ESTs, Highly similar to ALU2_HUMAN ALU SUBFAMILY SB SEQUENCE |
| | | | CONTAMINATION WARNING ENTRY [H. sapiens] |
| 6 | AA037675 | Hs.152675 | ESTs |
| 6 | N72794 | Hs.37716 | hypothetical protein MGC39320 |
| 6 | AI653672 | Hs.377610 | PNAS-123 |
| 6 | BE091833 | NA | gb: IL2-BT0731-260400-076-F04 BT0731 Homo sapiens cDNA, mRNA sequence |
| 6 | AA854133 | Hs.310462 | ESTs |
| 7 | AW511255 | NA | ESTs |
| 7 | AW182924 | Hs.128790 | ESTs |
| 7 | AW197644 | Hs.19107 | ESTs |
| 7 | AA215404 | Hs.355588 | ESTs |
| 7 | T82331 | Hs.31314 | calmodulin 2 (phosphorylase kinase, delta) |
| 7 | AI634046 | Hs.195175 | CASP8 and FADD-like apoptosis regulator |
| 7 | AA421020 | Hs.208919 | ESTs |
| 7 | AI932995 | Hs.183475 | Homo sapiens clone 25061 mRNA sequence |
| 7 | AA579297 | Hs.26937 | brain and nasopharyngeal carcinoma susceptibility protein |
| 7 | AA831815 | Hs.370756 | ESTs, Weakly similar to I78885 serine/threonine-specific protein kinase [H. sapiens] |
| 7 | AI732132 | Hs.109426 | ESTs |
| 7 | T85301 | Hs.88974 | gb: yd78d06.s1 Soares fetal liver spleen 1NFLS Homo sapiens cDNA clone 3′ similar to |
| | | | contains Alu repetitive element;, mRNA sequence |
| 7 | AI076259 | Hs.371556 | ESTs |
| 7 | AW979249 | NA | gb: EST391359 MAGE resequences, MAGP Homo sapiens cDNA, mRNA sequence |
| 7 | AW298359 | Hs.221069 | ESTs |
| 7 | Z48633 | Hs.283742 | H. sapiens mRNA for retrotransposon |
| 7 | T92576 | Hs.191168 | ESTs |
| 7 | AI638706 | Hs.405567 | ESTs, Weakly similar to A47582 B-cell growth factor precursor [H. sapiens] |
| 7 | BE158006 | Hs.212296 | ESTs |
| 7 | AF009267 | Hs.102238 | Homo sapiens clone FBA1 Cri-du-chat region mRNA |
| 8 | NM_030929.2 | NA | NM_030929.2\|Homo sapiens hypothetical protein FKSG28 (FKSG28), mRNA |
| 8 | NA | NA | Target Exon |
| 8 | AI307226 | Hs.164421 | ESTs |
| 8 | AA135159 | Hs.203349 | Homo sapiens cDNA FLJ12149 fis, clone MAMMA1000421 |
| 8 | AI277367 | Hs.47094 | ESTs |
| 8 | BE169995 | Hs.180799 | hypothetical protein FLJ22561 |
| 8 | AW958181 | Hs.189998 | ESTs |
| 8 | R08950 | Hs.272044 | ESTs, Weakly similar to ALU1_HUMAN ALU SUBFAMILY J SEQUENCE |
| | | | CONTAMINATION WARNING ENTRY [H. sapiens] |
| 8 | N58885 | Hs.289061 | gb: yy60a09.s1 Soares_multiple_sclerosis_2NbHMSP Homo sapiens cDNA clone 3′, |
| | | | mRNA sequence |
| 8 | AA215539 | Hs.283643 | Homo sapiens cDNA FLJ11606 fis, clone HEMBA1003942 |
| 8 | AA215701 | Hs.186541 | ESTs, Weakly similar to I38022 hypothetical protein [H. sapiens] |
| 8 | AA315703 | Hs.199993 | ESTs, Weakly similar to ALUB_HUMAN !!!! ALU CLASS B WARNING ENTRY !!! |
| | | | [H. sapiens] |
| 8 | AW936874 | NA | gb: RC1-DT0029-120100-011-f07 DT0029 Homo sapiens cDNA, mRNA sequence |
| 8 | H84455 | Hs.40639 | ESTs |
| 8 | BE549205 | Hs.184488 | flotillin 2 |
| 8 | AA971576 | Hs.225951 | topoisomerase-related function protein 4-1 |
| 8 | AW276866 | Hs.192715 | ESTs |
| 8 | AL047879 | Hs.293865 | ESTs, Weakly similar to ALU2_HUMAN ALU SUBFAMILY SB SEQUENCE |
| | | | CONTAMINATION WARNING ENTRY [H. sapiens] |
| 8 | AA657494 | NA | gb: nt66f04.s1 NCI_CGAP_Pr3 Homo sapiens cDNA clone similar to gb: M35663 |
| | | | INTERFERON-INDUCED, DOUBLE-STRANDED RNA-ACTIVATED PROTEIN |
| | | | KINASE (HUMAN);, mRNA sequence |
| 8 | AA699325 | Hs.269880 | ESTs |
| 8 | AW510927 | Hs.371883 | ESTs |
| 8 | AU077018 | Hs.3235 | keratin 4 |
| 8 | AA761490 | Hs.351250 | ESTs, Moderately similar to S65657 alpha-1C-adrenergic receptor splice form 2 |
| | | | [H. sapiens] |
| 8 | AW979008 | Hs.30738 | hypothetical protein FLJ10407 |
| 8 | AL045620 | Hs.131021 | hypothetical protein DKFZp434G118 |
| 8 | AW450681 | Hs.224941 | ESTs |
| 8 | N71597 | Hs.29698 | ESTs, Weakly similar to ZN91_HUMAN ZINC FINGER PROTEIN 91 [H. sapiens] |
| 8 | U54727 | Hs.191445 | ESTs |
| 8 | AW891965 | Hs.367942 | histone deacetylase 3 |
| 9 | NA | NA | C6001282: gi\|4504223\|ref\|NP_000172.1\|glucuronidase, beta [Homo sapiens] |
| | | | gi\|114963\|sp\|P082 |
| 9 | NM_138295.1 | NA | NM_138295.1\|Homo sapiens polycystic kidney disease 1 like 1 (PKD1L1), mRNA |
| 9 | X15673 | NA | gb: Human pTR2 mRNA for repetitive sequence. |
| 9 | AA031663 | Hs.28802 | centaurin-alpha 2 protein |
| 9 | AW971350 | Hs.63386 | ESTs |
| 9 | AW085690 | Hs.63428 | ESTs, Weakly similar to Z195_HUMAN ZINC FINGER PROTEIN 195 [H. sapiens] |
| 9 | AA079229 | NA | gb: zm95f04.r1 Stratagene colon HT29 (937221) Homo sapiens cDNA clone 5′ similar to |
| | | | gb: J03626 URIDINE 5′-MONOPHOSPHATE SYNTHASE (HUMAN);, mRNA sequence |
| 9 | AA205850 | Hs.122823 | thousand and one amino acid protein kinase |
| 9 | BE152644 | NA | gb: CM1-HT0329-250200-128-f09 HT0329 Homo sapiens cDNA, mRNA sequence |
| 9 | AA311223 | Hs.283091 | found in inflammatory zone 3 |
| 9 | AI052628 | Hs.271570 | ESTs, Weakly similar to 2109260A B cell growth factor [H. sapiens] |
| 9 | AA192455 | Hs.22968 | Homo sapiens clone IMAGE: 451939, mRNA sequence |
| 9 | R59096 | Hs.279939 | mitochondrial carrier homolog 1 |
| 9 | U38847 | Hs.151518 | TAR (HIV) RNA-binding protein 1 |
| 9 | AW938336 | Hs.193767 | ESTs |
| 9 | AI343641 | Hs.185798 | ESTs |
| 9 | AB007867 | Hs.278311 | plexin B1 |
| 9 | N52821 | Hs.269412 | ESts, Moderately similar to ALU7_HUMAN ALU SUBFAMILY SQ SEQUENCE |
| | | | CONTAMINATION WARNING ENTRY [H. sapiens] |
| 9 | AW972689 | Hs.200934 | ESTs |
| 9 | AA533447 | Hs.169610 | CD44 antigen (homing function and Indian blood group system) |
| 9 | AI056872 | Hs.133386 | ESTs |
| 9 | AA909619 | Hs.112668 | ESTs |
| 9 | AA736872 | Hs.371634 | ESTs |
| 9 | R97804 | Hs.18723 | ESTs |
| 9 | AA699991 | Hs.375200 | gb: zi69a09.s1 Soares_fetal_liver_spleen_1NFLS_S1 Homo sapiens cDNA clone 3′ similar |
| | | | to contains Alu repetitive element;, mRNA sequence |
| 9 | AI248285 | Hs.118348 | ESTs |
| 9 | AI640635 | Hs.116468 | EST |
| 9 | BE177778 | Hs.378703 | gb: RC1-HT0598-310300-012-f07 HT0598 Homo sapiens cDNA, mRNA sequence |
| 9 | AA897108 | NA | gb: am08a06.s1 Soares_NFL_T_GBC_S1 Homo sapiens cDNA clone 3′, mRNA sequence |
| 9 | BE327015 | Hs.81988 | disabled homolog 2, mitogen-responsive phosphoprotein (Drosophila) (DAB2), mRNA. |
| 9 | AI125436 | Hs.405924 | ESTs |
| 9 | BE562611 | Hs.348711 | gb: 601336446F1 NIH_MGC_44 Homo sapiens cDNA clone 5′, mRNA sequence |
| 9 | AI084182 | Hs.370293 | Homo sapiens cDNA FLJ14209 fis, clone NT2RP3003346 |
| 9 | B037731 | Hs.7871:65 | hypothetical protein FLJ10081 |
| 9 | AI222165 | Hs.144923 | ESTs |
| 9 | AV654627 | Hs.271808 | ESTs, Weakly similar to I38022 hypothetical protein [H. sapiens] |
| 9 | AW297283 | Hs.192819 | ESTs |
| 9 | AI762475 | Hs.151327 | ESTs, Moderately similar to ALU1_HUMAN ALU SUBFAMILY J SEQUENCE |
| | | | CONTAMINATION WARNING ENTRY [H. sapiens] |
| 9 | AF263462 | Hs.18376 | KIAA1319 protein |
| 9 | AI493546 | Hs.194737 | KIAA0453 protein |
| 9 | BE395253 | Hs.30861 | hypothetical protein MGC29956 (MGC29956), mRNA. |
| 9 | AW450536 | Hs.209260 | ESTs |
| 9 | R35917 | Hs.301338 | hypothetical protein FLJ12587 |
| 9 | AA748418 | Hs.33368 | hypothetical protein FLJ11175 |
| 9 | AA086123 | Hs.317177 | ESTs |
| 9 | AA721140 | NA | ESTs, Weakly similar to putative p150 [H. sapiens] |
| 9 | AW892049 | NA | gb: RC5-NT0035-260400-021-D11 NT0035 Homo sapiens cDNA, mRNA sequence |
| 9 | AI279811 | Hs.298553 | Homo sapiens, clone IMAGE: 3953631, mRNA, partial cds |
| 9 | BE160204 | Hs.390799 | gb: QV1-HT0413-010200-059-g08 HT0413 Homo sapiens cDNA, mRNA sequence |
| 10 | NM_005936 | NA | NM_005936: Homo sapiens myeloid/lymphoid or mixed-lineage leukemia (trithorax |
| | | | (Drosophila) homolog); translocated to, 4 (MLLT4), mRNA. |
| 10 | AA508857 | Hs.369326 | ESTs, Weakly similar to ALU1_HUMAN ALU SUBFAMILY J SEQUENCE |
| | | | CONTAMINATION WARNING ENTRY [H. sapiens] |
| 10 | AA724738 | Hs.131034 | ESTs, Weakly similar to 178885 serine/threonine-specific protein kinase [H. sapiens] |
| 10 | AA130992 | Hs.2794 | gb: zo15e02.s1 Stratagene colon (937204) Homo sapiens cDNA clone 3′ similar to |
| | | | contains Alu repetitive element; contains element PTR5 repetitive element;, mRNA |
| | | | sequence |
| 10 | AA160363 | Hs.269956 | ESTs |
| 10 | H69480 | Hs.141304 | ESTs |
| 10 | AI080042 | Hs.377298 | ribosomal protein S24 |
| 10 | BE549343 | Hs.82208 | acyl-Coenzyme A dehydrogenase, very long chain |
| 10 | AW967054 | Hs.206312 | ESTs, Weakly similar to I38022 hypothetical protein [H. sapiens] |
| 10 | AI821614 | Hs.87409 | ESTs |
| 10 | AA811933 | Hs.104234 | ESTs |
| 10 | AK000753 | Hs.92374 | hypothetical protein |
| 10 | AA811657 | Hs.220913 | ESTs |
| 10 | AI199510 | Hs.267912 | ESTs, Weakly similar to ALU7_HUMAN ALU SUBFAMILY SQ SEQUENCE |
| | | | CONTAMINATION WARNING ENTRY [H. sapiens] |
| 10 | AW469240 | NA | ESTs |
| 10 | AW970512 | NA | gb: EST382593 MAGE resequences, MAGK Homo sapiens cDNA, mRNA sequence |
| 10 | AW057782 | Hs.293053 | ESTs |
| 10 | AI868634 | Hs.246358 | ESTs, Weakly similar to T32250 hypothetical protein T15B7.3 - Caenorhabditis elegans |
| | | | [C. elegans] |
| 10 | BE300073 | Hs.279860 | tumor protein, translationally-controlled 1 |
| 10 | AA641201 | Hs.222051 | ESTs |
| 10 | AL118754 | NA | gb: DKFZp761P1910_r1 761 (synonym: hamy2) Homo sapiens cDNA clone |
| | | | DKFZp761P1910 5′, mRNA sequence |
| 10 | BE503432 | Hs.284153 | Fanconi anemia, complementation group A |
| 10 | AB002375 | Hs.156814 | KIAA0377 gene product |
| 10 | AA632817 | Hs.190316 | ESTs |
| 10 | AA372796 | NA | ESTs, Weakly similar to AF161356 1 HSPC093 [H. sapiens] |
| 10 | AK001016 | Hs.356519 | hypothetical protein FLJ10154 |
| 10 | AI553741 | Hs.98791 | ESTs |
| 10 | AW369620 | Hs.33944 | ESTs, Weakly similar to ALU1_HUMAN ALU SUBFAMILY J SEQUENCE |
| | | | CONTAMINATION WARNING ENTRY [H. sapiens] |
| 10 | AA459316 | Hs.99743 | ESTs |
| 10 | AW967807 | Hs.13797 | ESTs |
| 10 | AW972227 | Hs.163986 | Homo sapiens cDNA: FLJ22765 fis, clone KAIA1180 |
| 10 | AW972771 | Hs.292471 | ESTs, Weakly similar to ALU1_HUMAN ALU SUBFAMILY J SEQUENCE |
| | | | CONTAMINATION WARNING ENTRY [H. sapiens] |
| 10 | AI131140 | Hs.372186 | ESTs |
| 10 | AA570710 | Hs.349344 | hypothetical protein BC001573 |
| 10 | AA832055 | NA | ESTs, Weakly similar to ALU1_HUMAN ALU SUBFAMILY J SEQUENCE |
| | | | CONTAMINATION WARNING ENTRY [H. sapiens] |
| 10 | AA604405 | NA | gb: no87h09.s1 NCI_CGAP_AA1 Homo sapiens cDNA clone 3′, mRNA sequence |
| 10 | AI174777 | Hs.400372 | Homo sapiens PRO2492 mRNA, complete cds |
| 10 | AI611172 | Hs.189578 | ESTs |
| 10 | AA460479 | Hs.321707 | KIAA0742 protein |
| 10 | AI378570 | Hs.116397 | ESTs |
| 10 | AA648983 | Hs.370514 | ESTs |
| 10 | AI285970 | Hs.183817 | ESTs |
| 10 | AW015736 | Hs.211378 | ESTs |
| 10 | T97301 | Hs.18026 | ESTs |
| 10 | BE301871 | Hs.4867 | mannosyl (alpha-1,3-)-glycoprotein beta-1,4-N-acetylglucosaminyltransferase, isoenzyme B |
| 10 | AW021655 | Hs.194441 | ESTs |
| 10 | AF220263 | Hs.193920 | MOST2 protein |
| 10 | W90446 | Hs.137324 | ESTs |
| 10 | AI418466 | Hs.33665 | ESTs |
| 10 | AA704899 | Hs.291651 | ESTs, Weakly similar to I38022 hypothetical protein [H. sapiens] |
| 10 | AI433540 | Hs.405182 | gb: ti69g05.x1 NCI_CGAP_Kid11 Homo sapiens cDNA clone 3′, mRNA sequence |
| 10 | R55822 | Hs.4268 | ESTs |
| 10 | AA810788 | Hs.123337 | ESTs |
| 10 | AI660898 | Hs.119533 | ESTs |
| 10 | AL138461 | Hs.323084 | tRNA-guanine transglycosylase |
| 10 | AI570700 | Hs.128025 | ESTs |
| 10 | BE244622 | Hs.8084 | hypothetical protein dJ465N24.2.1 |
| 10 | AA983913 | Hs.368672 | ESTs |
| 10 | AA355525 | Hs.159604 | cysteinyl-tRNA synthetase |
| 10 | AI025499 | Hs.370408 | ESTs |
| 10 | AI280341 | Hs.166571 | ESTs |
| 10 | AV651680 | Hs.208558 | ESTs |
| 10 | AI674383 | Hs.22891 | solute carrier family 7 (cationic amino acid transporter, y system), member 8 |
| 10 | R07355 | Hs.15464 | Homo sapiens cDNA: FLJ21351 fis, clone COL02762 |
| 10 | AI733819 | Hs.145557 | ESTs |
| 10 | AL137730 | Hs.14235 | hypothetical protein FLJ20008; KIAA1839 protein |
| 10 | AW205632 | Hs.211198 | ESTs |
| 10 | AI962234 | Hs.196102 | ESTs |
| 10 | AI651803 | Hs.370331 | ESTs |
| 10 | R94570 | Hs.266869 | ESTs, Weakly similar to ALU1_HUMAN ALU SUBFAMILY J SEQUENCE |
| | | | CONTAMINATION WARNING ENTRY [H. sapiens] |
| 10 | AI540842 | Hs.61082 | ESTs |
| 10 | AW838616 | Hs.372534 | gb: RC5-LT0054-140200-013-D01 LT0054 Homo sapiens cDNA, mRNA sequence |
| 11 | NA | NA | Target Exon |
| 11 | AA045899 | Hs.146170 | hypothetical protein FLJ22969 |
| 11 | T82427 | Hs.194101 | Homo sapiens cDNA: FLJ20869 fis, clone ADKA02377 |
| 11 | AU077343 | Hs.43910 | CD164 antigen, sialomucin |
| 11 | AW206670 | Hs.50748 | chromosome 21 open reading frame 18 |
| 11 | AA525225 | Hs.334630 | Homo sapiens cDNA FLJ14462 fis, clone MAMMA1000241 |
| 11 | BE181659 | NA | gb: QV1-HT0638-070500-191-g07 HT0638 Homo sapiens cDNA, mRNA sequence |
| 11 | BE327036 | Hs.172813 | Rho guanine nucleotide exchange factor (GEF) 7 (ARHGEF7), transcript variant 1, |
| | | | mRNA. |
| 11 | AF022375 | Hs.73793 | vascular endothelial growth factor |
| 11 | AA456195 | Hs.10056 | hypothetical protein FLJ14621 |
| 11 | N92571 | Hs.54808 | ESTs |
| 11 | L19067 | Hs.75569 | v-rel avian reticuloendotheliosis viral oncogene homolog A (nuclear factor of kappa light |
| | | | polypeptide gene enhancer in B-cells 3 (p65)) |
| 11 | AW938668 | NA | gb: PMI-DT0063-160200-003-c07 DT0063 Homo sapiens cDNA, mRNA sequence |
| 11 | AW452420 | Hs.248678 | ESTs |
| 11 | T77127 | Hs.375694 | gb: yd72a05.r1 Soares fetal liver spleen 1NFLS Homo sapiens cDNA clone 5′, mRNA |
| | | | sequence |
| 11 | R94977 | Hs.35416 | PRO0132 protein |
| 11 | AA229781 | Hs.336812 | ESTs |
| 11 | AJ224901 | Hs.109526 | zinc finger protein 198 |
| 11 | AA016188 | Hs.111244 | hypothetical protein |
| 11 | AV647015 | Hs.349256 | paired immunoglobulin-like receptor beta |
| 11 | NM_004428 | Hs.1624 | ephrin-A1 |
| 11 | BE244625 | Hs.125742 | leucine-rich neuronal protein |
| 11 | AA505691 | Hs.145696 | splicing factor (CC1.3) |
| 11 | AA469042 | Hs.164410 | chromosome 16 open reading frame 7 |
| 11 | AA494172 | Hs.194417 | ESTs |
| 11 | BE397531 | Hs.182237 | POU domain, class 2, transcription factor 1 |
| 11 | AW969656 | NA | gb: EST381733 MAGE resequences, MAGK Homo sapiens cDNA, mRLNA sequence |
| 11 | AL023754 | Hs.199068 | similar to calcium/calmodulin dependent protein kinases |
| 11 | AW793022 | Hs.323463 | hypothetical protein |
| 11 | AA487264 | Hs.154974 | Homo sapiens mRNA; cDNA DKFZp667N064 (from clone DKFZp667N064) |
| 11 | AI874223 | Hs.293560 | ESTs |
| 11 | AA761378 | Hs.192013 | ESTs |
| 11 | AK000777 | Hs.272197 | Homo sapiens cDNA FLJ20770 fis, clone COL06509 |
| 11 | R31178 | Hs.287820 | fibronectin 1 |
| 11 | AL043683 | Hs.8173 | hypothetical protein FLJ10803 |
| 11 | BE242758 | Hs.190223 | ESTs, Moderately similar to T29285 hypothetical protein C34D4.I4 Caenorhabditis |
| | | | elegans [C. elegans] |
| 11 | AI674779 | Hs.126744 | ESTs |
| 11 | AA586950 | Hs.373755 | Homo sapiens mRNA; cDNA DKFZp761G18121 (from clone DKFZp761G18121); |
| | | | complete cds |
| 11 | AW273261 | Hs.216292 | ESTs |
| 11 | BE005398 | Hs.375092 | gb: CM1-BN0116-150400-189-h02 BN0116 Homo sapiens cDNA, mRNA sequence |
| 11 | T51910 | Hs.9333 | ESTs |
| 11 | AL042425 | Hs.283976 | hypthetical protein PRO2389 |
| 11 | AW975684 | Hs.294014 | ESTs |
| 11 | AA745618 | Hs.110613 | BANP homolog, SMAR1 homolog |
| 11 | AA279341 | Hs.174151 | aldehyde oxidase 1 |
| 11 | AW753588 | Hs.86998 | Homo sapiens cDNA FLJ10205 fis, clone HEMBA1004954 |
| 11 | AI954880 | Hs.372464 | ESTs |
| 11 | AW609170 | Hs.398050 | ESTs |
| 11 | AI420611 | Hs.153934 | core-binding factor, runt domain, alpha subunit 2; translocated to, 2 |
| 11 | AI887875 | Hs.307434 | ESTs |
| 11 | H15560 | Hs.131833 | ESTs |
| 11 | AI038316 | Hs.156317 | gb: ox48c08.x1 Soares_total_fetus_Nb2HF8_9w Homo sapiens cDNA clone 3′, mRNA |
| | | | sequence |
| 11 | T47764 | Hs.132917 | ESTs |
| 11 | R69077 | Hs.193348 | ESTs, Moderately similar to 178885 serine/threonine-specific protein kinase [H. sapiens] |
| 11 | AI073491 | Hs.269887 | ESTs, Highly similar to KPBB_HUMAN PHOSPHORYLASE B KINASE BETA |
| | | | REGULATORY CHAIN [H. sapiens] |
| 11 | R44284 | Hs.2730 | heterogeneous nuclear ribonucleoprotein L |
| 11 | AW594695 | Hs.167046 | ESTs |
| 11 | AI679753 | Hs.371392 | ESTs, Weakly similar to ALU7_HUMAN ALU SUBFAMILY SQ SEQUENCE |
| | | | CONTAMINATION WARNING ENTRY [H. sapiens] |
| 11 | H22953 | Hs.137551 | ESTs |
| 11 | BE546846 | Hs.195048 | ESTs |
| 11 | AA010200 | Hs.175551 | ESTs |
| 11 | T98171 | Hs.185675 | ESTs |
| 11 | AA046457 | Hs.60677 | ESTs |
| 11 | AW102941 | Hs.211265 | ESTs |
| 11 | AA025386 | Hs.61311: 24 | ESTs, Weakly similar to S10590 cysteine proteinase [H. sapiens] |
| 11 | AF044924 | Hs.30792 | hook2 protein |
| 11 | R41874 | Hs.22164 | AD038 |
| 11 | AI978583 | Hs.329273 | ESTs, Weakly similar to 178885 serine/threonine-specific protein kinase [H. sapiens] |
| 11 | BE620712 | Hs.33026 | hypothetical protein PP2447 |
| 11 | AW362901 | Hs.68864 | lipase, member H (LIPH), mRNA. |
| 11 | AI905216 | NA | gb: RC-BT078-260499-024 BT078 Homo sapiens cDNA, mRNA sequence |
| 11 | AA889982 | Hs.271826 | ESTs, Weakly similar to I38022 hypothetical protein [H. sapiens] |
| 11 | AA320038 | NA | gb: EST22383 Adipose tissue, white II Homo sapiens cDNA 5′ end, mRNA sequence |
| 12 | M22333 | NA | Target Exon |
| 12 | H90988 | Hs.334503 | hypothetical protein MGC12386 |
| 12 | AA194952 | Hs.36093 | Homo sapiens cDNA FLJ12885 fis, clone NT2RP2003988 |
| 12 | AI860558 | Hs.62112 | zinc finger protein 207 |
| 12 | AA378739 | Hs.187711 | ESTs |
| 12 | AW511443 | Hs.258110 | ESTs |
| 12 | AF075113 | Hs.384696 | gb: Homo sapiens full length insert cDNA YU78B07 |
| 12 | AI357813 | Hs.239926 | sterol-C4-methyl oxidase-like |
| 12 | AW607444 | Hs.134622 | ESTs |
| 12 | AW265634 | Hs.133100 | ESTs |
| 12 | AI827988 | Hs.240728 | ESTs, Moderately similar to PC4259 ferritin associated protein [H. sapiens] |
| 12 | AW340925 | Hs.110855 | ESTs |
| 12 | N72596 | NA | gb: za46f04.s1 Soares fetal liver spleen 1NFLS Homo sapiens cDNA clone 3′ similar to |
| | | | SW: PL10_MOUSE P16381 PUTATIVE ATP-DEPENDENT RNA HELICASE PL10. [1];, |
| | | | mRNA sequence |
| 13 | AI125507 | Hs.130829 | transformer-2 alpha (htra-2 alpha) |
| 13 | AA534222 | NA | gb: nj21d02.s1 NCI_CGAP_AA1 Homo sapiens cDNA clone 3′ similar to contains Alu |
| | | | repetitive element;, mRNA sequence |
| 13 | AW976511 | Hs.112592 | ESTs |
| 14 | AI801565 | Hs.200113 | Homo sapiens cDNA FLJ11379 fis, clone HEMBA1000469 |
| 14 | H13016 | Hs.198281 | pyruvate kinase, muscle |
| 14 | AA521132 | Hs.48576 | excision repair cross-complementing rodent repair deficiency, complementation group 5 |
| | | | (xeroderma pigmentosum, complementation group G (Cockayne syndrome)) |
| 14 | BE259015 | Hs.74576 | GDP dissociation inhibitor 1 |
| 14 | AI912061 | Hs.55016 | hypothetical protein FLJ21935 |
| 14 | AA093428 | Hs.352337 | ESTs |
| 14 | H70814 | Hs.23368 | Homo sapiens clone FLC0578 PRO2852 mRNA, complete cds |
| 14 | AA197305 | Hs.123075 | ESTs, Weakly similar to A46010 X-linked retinopathy protein [H. sapiens] |
| 14 | H77859 | Hs.377218 | reticulon 4 |
| 14 | AW449855 | Hs.96557 | Homo sapiens cDNA FLJ12727 fis, clone NT2RP2000027 |
| 14 | AI922821 | Hs.32433 | ESTs |
| 14 | BE281303 | Hs.299148 | hypothetical protein FLJ21801 |
| 14 | H82114 | Hs.74170 | ESTs |
| 14 | AI149880 | Hs.188809 | ESTs |
| 14 | AF169255 | Hs.241377 | 5-hydroxytryptamine (serotonin) receptor 3B |
| 14 | AI584156 | Hs.105640 | Homo sapiens, clone IMAGE: 4139775, mRNA, partial cds |
| 14 | NM_013937 | Hs.247861 | olfactory receptor, family 11, subfamily A, member 1 |
| 14 | AW023610 | Hs.370582 | ESTs |
| 14 | AA516420 | Hs.352340 | ESTs, Weakly similar I38022 hypothetical protein [H. sapiens] |
| 14 | NM_014159 | Hs.6947 | HSPC069 protein |
| 14 | AI658666 | Hs.352381 | RNA binding motif protein 4 |
| 14 | AA551569 | Hs.272034 | hypothetical protein PRO2822 |
| 14 | AA700439 | Hs.188490 | ESTs |
| 14 | BE326856 | Hs.118795 | hypothetical protein FLJ10008 |
| 14 | AW080237 | Hs.252884 | ESTs |
| 14 | AL137480 | Hs.6834 | KIAA1014 protein |
| 14 | BE559786 | Hs.375037 | hypothetical protein FLJ30092 |
| 14 | AW206035 | Hs.356457 | ESTs |
| 14 | AI743317 | Hs.283622 | ESTs, Weakly similar to ALU5_HUMAN ALU SUBFAMILY SC SEQUENCE |
| | | | CONTAMINATION WARNING ENTRY [H. sapiens] |
| 14 | AI923953 | Hs.131830 | ESTs |
| 14 | H80137 | Hs.157246 | ESTs |
| 14 | AA228092 | Hs.42656 | KIAA1681 protein |
| 14 | AI523875 | NA | gb: tg97d04.x1 NCI_CGAP_CLL1 Homo sapiens cDNA clone 3′ similar to contains Alu |
| | | | repetitive element; contains element THR THR repetitive element;, mRNA sequence |
| 14 | AI619957 | NA | ESTs |
| 14 | AA019344 | Hs.2055 | ubiquitin-activating enzyme E1 (A1S9T and BN75 temperature sensitivity |
| | | | complementing) |
| 14 | AF070582 | Hs.26118 | hypothetical protein MGC13033 |
| 14 | AF095687 | Hs.26937 | brain and nasopharyngeal carcinoma susceptibility protein |
| 14 | AW452189 | Hs.27263 | KIAA1458 protein |
| 14 | N58327 | Hs.302755 | ESTs |
| 15 | NA | NA | Target Exon |
| 15 | N33937 | Hs.10336 | ESTs |
| 15 | BE349470 | Hs.99918 | mucin 6, gastric |
| 15 | AW851603 | Hs.278831 | gb: MR2-CT0222-201099-001-f04 CT0222 Homo sapiens cDNA, mRNA sequence |
| 15 | BE091833 | NA | gb: IL2-BT0731-260400-076-F04 BT0731 Homo sapiens cDNA, mRNA sequence |
| 15 | BE156536 | Hs.6217 | gb: QV0-HT0368-310100-091-h10 HT0368 Homo sapiens cDNA, mRNA sequence |
| 15 | AW795793 | Hs.356181 | Homo sapiens cDNA FLJ12257 fis, clone MAMMA 1001501, highly similar to CALPAIN |
| | | | 1, LARGE [CATALYTIC] SUBUNIT (EC 3.4.22.17) |
| 15 | AW952192 | Hs.406618 | guanine nucleotide binding protein (G protein), alpha stimulating activity polypeptide 1 |
| 15 | AA962181 | Hs.111219 | ESTs, Moderately similar to ALU1_HUMAN ALU SUBFAMILY J SEQUENCE |
| | | | CONTAMINATION WARNING ENTRY [H. sapiens] |
| 15 | AA226377 | Hs.193950 | ESTs |
| 15 | AA317036 | Hs.301771 | transforming growth factor, beta-induced, 68 kD |
| 15 | T18988 | Hs.293668 | ESTs |
| 15 | AA482027 | Hs.142569 | ESTs, Weakly similar to I38022 hypothetical protein [H. sapiens] |
| 15 | AA521410 | Hs.41371 | ESTs |
| 15 | AW971248 | Hs.291289 | ESTs, Weakly similar to ALU1_HUMAN ALU SUBFAMILY J SEQUENCE |
| | | | CONTAMINATION WARNING ENTRY [H. sapiens] |
| 15 | AA502663 | Hs.145037 | ESTs |
| 15 | AA534908 | Hs.2860 | POU domain, class 5, transcription factor 1 |
| 15 | AA775208 | Hs.136423 | ESTs |
| 15 | AB029396 | Hs.381050 | beta-1,3-glucuronyltransferase 1 (glucuronosyltransferase P) |
| 15 | AW022133 | Hs.189838 | ESTs |
| 15 | AA608955 | Hs.109653 | ESTs |
| 15 | AI033647 | Hs.121001 | Homo sapiens, clone IMAGE: 3460280, mRNA |
| 15 | AA704806 | Hs.143842 | ESTs, Weakly similar to 2004399A chromosomal protein [H. sapiens] |
| 15 | AI690734 | Hs.62112 | Homo sapiens cDNA: FLJ22562 fis, clone HSI01814 |
| 15 | AL353957 | Hs.284181 | hypothetical protein DKFZp434P0531 |
| 15 | AA780020 | Hs.21320 | postreplication repair protein hRAD18p |
| 15 | H87407 | Hs.348407 | chorionic gonadotropin, beta polypeptide |
| 15 | AA833902 | Hs.270745 | ESTs |
| 15 | AA885234 | Hs.125774 | ESTs |
| 15 | AI792868 | Hs.135365 | ESTs |
| 15 | AI762154 | Hs.315054 | Homo sapiens cDNA FLJ14014 fis, clone HEMBA1000290 |
| 15 | AA010269 | Hs.16241 | ESTs |
| 15 | AW500269 | Hs.21264 | KIAA0782 protein |
| 15 | AL049390 | Hs.22689 | Homo sapiens mRNA; cDNA DKFZp586O1318 (from clone DKFZp586O1318) |
| 15 | AA011518 | Hs.271778 | ESTs, Weakly similar to I38022 hypothetical protein [H. sapiens] |
| 15 | AW451469 | Hs.209990 | ESTs |
| 15 | AW389509 | Hs.223747 | ESTs |
| 15 | AI924228 | Hs.115185 | ESTs, Moderately similar to PC4259 ferritin associated protein [H. sapiens] |
| 15 | AI821940 | Hs.72071 | hypothetical protein FLJ20038 |
| 15 | BE142728 | NA | gb: MR0-HT0157-021299-004-d08 HT0157 Homo sapiens cDNA, mRNA sequence |
| 16 | NM_020962.1 | NA | NM_020962.1\|Homo sapiens likely ortholog of mouse neighbor of Punc E11 (NOPE), |
| 16 | AJ234589.1 | NA | AJ237589.1\|HSA237589 Homo sapiens mRNA for T-box transcription factor (TBX20 |
| | | | gene), |
| 16 | AA386192 | Hs.193482 | Homo sapiens cDNA FLJ11903 fis, clone HEMBB1000030 |
| 16 | AA302840 | Hs.403902 | gb: EST10534 Adipose tissue, white I Homo sapiens cDNA 3′ end, mRNA sequence |
| 16 | AW515373 | Hs.271249 | Homo sapiens cDNA FLJ13580 fis, clone PLACE1008851 |
| 16 | AA136569 | Hs.356559 | KIAA0187 gene product |
| 16 | AI567436 | Hs.16258 | Homo sapiens cDNA FLJ11699 fis, clone HEMBA1005047, highly similar to RAS- |
| | | | RELATED PROTEIN RAB-24 |
| 16 | R43528 | Hs.388002 | ESTs |
| 16 | AA828750 | NA | gb: od76a07.s1 NCI_CGAP_Ov2 Homo sapiens cDNA clone, mRNA sequence |
| 16 | AA676544 | Hs.171545 | HIV-1 Rev binding protein |
| 16 | AW972872 | Hs.293736 | ESTs |
| 16 | AI670057 | Hs.199882 | ESTs |
| 16 | AF065215 | Hs.198161 | phospholipase A2, group IVB (cytosolic) |
| 16 | AA456883 | Hs.79889 | monocyte to macrophage differentiation-associated |
| 16 | R51790 | Hs.239483 | Human clone 23933 mRNA sequence |
| 16 | AA478883 | Hs.273766 | ESTs |
| 16 | AA572949 | Hs.207566 | ESTs |
| 16 | AW207279 | Hs.271786 | ESTs, Weakly similar to PC4395 mucin 3 [H. sapiens] |
| 16 | AF124150 | Hs.371417 | ESTs |
| 16 | AW203986 | Hs.213003 | ESTs |
| 16 | AW749865 | NA | ESTs, Weakly similar to I38022 hypothetical protein [H. sapiens] |
| 16 | T85104 | Hs.194477 | E3 ubiquitin ligase SMURF2 |
| 16 | AW238673 | Hs.146038 | ESTs |
| 16 | AI908538 | Hs.133000 | ESTs, Weakly similar to S26689 hypothetical protein hc1 - mouse [M. musculus] |
| 16 | AW771958 | Hs.175437 | ESTs, Moderately similar to PC4259 ferritin associated protein [H. sapiens] |
| 16 | AI766732 | Hs.210628 | ESTs |
| 16 | AI903313 | Hs.34579 | ESTs, Moderately similar to ALU6_HUMAN ALU SUBFAMILY SP SEQUENCE |
| | | | CONTAMINATION WARNING ENTRY [H. sapiens] |
| 16 | AW974642 | Hs.366446 | ESTs, Weakly similar to ALU1_HUMAN ALU SUBFAMILY J SEQUENCE |
| | | | CONTAMINATION WARNING ENTRY [H. sapiens] |
| 17 | D00159 | NA | gb: Homo sapiens gene for pancreatic elastase I, partial cds. |
| 17 | AI204033 | Hs.379039 | tumor suppressor deleted in oral cancer-related 1 |
| 17 | T40707 | Hs.270862 | ESTs |
| 17 | AW971303 | Hs.241869 | ESTs |
| 17 | AA320525 | Hs.201076 | ESTs |
| 17 | AL110203 | Hs.138411 | Homo sapiens mRNA; cDNA DKFZp586J1922 (from clone DKFZp586J1922) |
| 17 | AW970116 | Hs.310616 | ESTs |
| 17 | AW971146 | Hs.293187 | ESTs |
| 17 | T55958 | Hs.384169 | gb: yb35f05.r1 Stratagene fetal spleen (937205) Homo sapiens cDNA clone 5′, mRNA |
| | | | sequence |
| 17 | AW444619 | Hs.138211 | ESTs |
| 17 | AI239832 | Hs.15617 | ESTs, Weakly similar to ALU4_HUMAN ALU SUBFAMILY SB2 SEQUENCE |
| | | | CONTAMINATION WARNING ENTRY [H. sapiens] |
| 17 | T85314 | Hs.54629 | thioredoxin-like |
| 17 | R10799 | Hs.191990 | ESTs |
| 17 | W69171 | Hs.267263 | hypothetical protein FLJ22283 (FLJ22283), mRNA. |
| 18 | AA682384 | NA | ESTs |
| 19 | AW861225 | Hs.110613 | BANP homolog, SMAR1 homolog |
| 20 | BRCA1b | NA | Eos Control: |

TABLE 2
CLUSTER 1 GENES INDICATIVE OF COLORECTAL CANCER

| | Exemplar | | |
|---|---|---|---|
| Cluster | Accession | UniGene ID | UniGeneTitle |
| 1 | NA | Hs.76297 | G protein-coupled receptor kinase 6 (GPRK6), mRNA. |
| 1 | NM_173483 | NA | NM_173483 Homo sapiens hypothetical protein FLJ39501 (FLJ39501) |
| 1 | NM_003468.2 | NA | NM_003468.2\|Homo sapiens frizzled homolog 5 (Drosophila) (FZD5), mRNA |
| 1 | NA | NA | Target Exon |
| 1 | AC007050.25 | NA | ESTs |
| 1 | NA | NA | Target Exon |
| 1 | W25945 | Hs.8173 | hypothetical protein FLJ10803 |
| 1 | AW054922 | Hs.53478 | Homo sapiens cDNA FLJ12366 fis, clone MAMMA1002411 |
| 1 | AW847814 | Hs.289005 | Homo sapiens cDNA: FLJ21532 fis, clone COL06049 |
| 1 | BE244200 | Hs.406243 | KIAA0410 gene product |
| 1 | AW514668 | Hs.194258 | ESTs, Moderately similar to ALU5_HUMAN ALU SUBFAMILY SC SEQUENCE |
| | | | CONTAMINATION WARNING ENTRY [H. sapiens] |
| 1 | AA249096 | Hs.32793 | ESTs |
| 1 | L26953 | Hs.1010 | regulator of mitotic spindle assembly 1 |
| 1 | AI381687 | Hs.404198 | ESTs |
| 1 | N99638 | Hs.87409 | gb: za39g11.r1 Soares fetal liver spleen 1NFLS Homo sapiens cDNA clone 5′ similar to |
| | | | contains Alu repetitive element;, mRNA sequence |
| 1 | AI205785 | Hs.190153 | ESTs |
| 1 | AW965212 | Hs.278871 | hypothetical protein FLJ30921 (FLJ30921), mRNA. |
| 1 | AL119442 | Hs.380968 | eukaryotic translation initiation factor 4 gamma, 2 |
| 1 | AA358045 | NA | gb: EST66944 Fetal lung III Homo sapiens cDNA 5′ end similar to EST containing Alu repeat, |
| | | | mRNA sequence |
| 1 | AL050276 | Hs.159456 | zinc finger protein 288 |
| 1 | AI052358 | Hs.131741 | ESTs |
| 1 | AW976570 | Hs.97387 | ESTs |
| 1 | AI936504 | Hs.2083 | CDC-like kinase 1 |
| 1 | AA400079 | Hs.257854 | ESTs |
| 1 | AW883367 | Hs.356546 | hypothetical protein MGC5306 |
| 1 | AA417696 | Hs.372121 | ESTs |
| 1 | AA470152 | Hs.368209 | ESTs |
| 1 | AW971375 | Hs.292921 | ESTs |
| 1 | AW971070 | Hs.291160 | ESTs, Weakly similar to ALU1_HUMAN ALU SUBFAMILY J SEQUENCE |
| | | | CONTAMINATION WARNING ENTRY [H. sapiens] |
| 1 | T87431 | Hs.190738 | ESTs |
| 1 | AA531129 | Hs.190297 | ESTs |
| 1 | AW439330 | Hs.256889 | ESTs, Weakly similar to 2109260A B cell growth factor [H. sapiens] |
| 1 | AW157424 | Hs.280685 | ESTs, Weakly similar to 138022 hypothetical protein [H. sapiens] |
| 1 | AB040966 | Hs.83575 | KIAA1533 protein |
| 1 | AW188370 | Hs.250383 | Homo sapiens cDNA FLJ14279 fis, clone PLACE1005574 |
| 1 | AA628539 | Hs.57783 | Homo sapiens eukaryotic translation initiation factor 3, subunit 9 eta, 116 kDa (EIF3S9) |
| 1 | AA640770 | Hs.200994 | EST |
| 1 | AA664078 | NA | gb: ac04a05.s1 Stratagene lung (937210) Homo sapiens cDNA clone 3′ similar to contains Alu |
| | | | repetitive element;, mRNA sequence |
| 1 | AA886511 | Hs.189282 | Homo sapiens cDNA: FLJ21429 fis, clone COL04205 |
| 1 | AA830893 | Hs.119769 | ESTs |
| 1 | BE327477 | Hs.166941 | ESTs |
| 1 | AI821940 | Hs.72071 | hypothetical protein FLJ20038 |
| 1 | AL137723 | Hs.5855 | Homo sapiens mRNA; cDNA DKFZp434D0818 (from clone DKFZp434D0818) |
| 1 | AA769874 | Hs.155287 | ubiquitin-protein isopeptide ligase (E3) |
| 1 | AI126162 | Hs.129037 | ESTs |
| 1 | AW748336 | Hs.168052 | KIAA0421 protein |
| 1 | AW083789 | Hs.124620 | ESTs |
| 1 | AI034357 | Hs.211194 | ESTs, Weakly similar to ALU8_HUMAN ALU SUBFAMILY SX SEQUENCE |
| | | | CONTAMINATION WARNING ENTRY [H. sapiens] |
| 1 | AW827419 | Hs.144139 | ESTs |
| 1 | BE262656 | Hs.32603 | hypothetical protein MGC3279 similar to collectins |
| 1 | AW469180 | Hs.346398 | ESTs |
| 1 | AI492857 | NA | gb: th72h08.x1 Soares_NhHMPu_S1 Homo sapiens cDNA clone 3′, mRNA sequence |
| 1 | AW451347 | Hs.175862 | ESTs |
| 1 | AI698091 | Hs.107845 | ESTs |
| 1 | AJ010046 | Hs.25155 | neuroepithelial cell transforming gene 1 |
| 1 | AL043983 | Hs.125063 | Homo sapiens cDNA FLJ13825 fis, clone THYRO1000558 |
| 1 | AW382884 | Hs.5320 | ESTs |
| 1 | BE378541 | Hs.279815 | cysteine sulfinic acid decarboxylase-relatedprotein 2 |
| 1 | R66282 | Hs.20247 | ESTs, Weakly similar to S65657 alpha-1C-adrenergic receptor splice form 2 [H. sapiens] |
| 1 | BE086548 | Hs.42346 | calcineurin-binding protein calsarcin-1 |
| 1 | AA907305 | Hs.36475 | ESTs |

TABLE 3
CLUSTER 4 GENES INDICATIVE OF METASTATIC COLORECTAL CANCER

| | Exemplar | | |
|---|---|---|---|
| Cluster | Accession | UniGene ID | UniGeneTitle |
| 4 | AA130986 | Hs.271627 | ESTs |
| 4 | T64896 | Hs.406798 | Homo sapiens cDNA FLJ11533 fis, clone HEMBA1002678 |
| 4 | AA132637 | Hs.15396 | Homo sapiens, clone IMAGE: 3948909, mRNA, partial cds |
| 4 | AA317962 | Hs.249721 | ESTs, Moderately similar to PC4259 ferritin associated protein [H. sapiens] |
| 4 | AW167439 | Hs.190651 | Homo sapiens cDNA FLJ13625 fis, clone PLACE1011032 |
| 4 | AW452823 | Hs.135268 | ESTs |
| 4 | AA132255 | Hs.143951 | ESTs |
| 4 | D83782 | Hs.78442 | SREBP CLEAVAGE-ACTIVATING PROTEIN |
| 4 | AI690465 | Hs.201661 | ESTs, Weakly similar to JC5238 galactosylceramide-like protein, GCP [H. sapiens] |
| 4 | R07785 | Hs.429867 | ESTs |
| 4 | AL041465 | Hs.182982 | golgin-67 |
| 4 | AW183695 | Hs.370907 | ESTs |
| 4 | AW276914 | Hs.423341 | Homo sapiens clone IMAGE: 713177, mRNA sequence |
| 4 | U50535 | Hs.110630 | Human BRCA2 region, mRNA sequence CG006 |
| 4 | AF073931 | Hs.122359 | calcium channel, voltage-dependent, alpha 1H subunit |
| 4 | AW341131 | Hs.146345 | ESTs |
| 4 | BE176694 | Hs.279860 | tumor protein, translationally-controlled 1 |
| 4 | AW963118 | Hs.161784 | ESTs |
| 4 | AW513691 | Hs.270149 | ESTs, Weakly similar to 2109260A B cell growth factor [H. sapiens] |
| 4 | BE173380 | Hs.381903 | ESTs |
| 4 | Z29067 | Hs.2236 | NIMA (never in mitosis gene a)-related kinase 3 |
| 4 | AA425310 | Hs.155766 | ESTs, Weakly similar to A47582 B-cell growth factor precursor [H. sapiens] |
| 4 | AW973253 | Hs.292689 | ESTs |
| 4 | AA453987 | Hs.144802 | ESTs |
| 4 | AA612710 | Hs.284148 | ESTs |
| 4 | AA830335 | Hs.105273 | ESTs |
| 4 | AW970859 | Hs.313503 | ESTs |
| 4 | AA532718 | HS.178604 | ESTs |
| 4 | AI459519 | Hs.314437 | clone IMAGE: 4607209, mRNA sequence [H. sapiens] |
| 4 | BE263901 | Hs.381222 | ESTs, Weakly similar to S37431 ankyrin 2, neuronal long splice form [H. sapiens] |
| 4 | AI301080 | Hs.35276 | KIAA0852 protein |
| 4 | AW975009 | Hs.292274 | ESTs, Weakly similar to A46010 X-linked retinopathy protein [H. sapiens] |
| 4 | AA677540 | Hs.117064 | ESTs |
| 4 | H74319 | Hs.188620 | ESTs |
| 4 | AI800041 | Hs.369733 | ESTs |
| 4 | AL360140 | Hs.176005 | Homo sapiens mRNA full length insert cDNA clone EUROIMAGE 113222 |
| 4 | AF134160 | Hs.7327 | claudin 1 |
| 4 | AI982794 | Hs.159473 | ESTs |
| 4 | AK001631 | Hs.8083 | hypothetical protein FLJ10769 |
| 4 | W22152 | Hs.282929 | ESTs |
| 4 | H77824 | NA | ESTs |
| 4 | AU076643 | Hs.313 | secreted phosphoprotein 1 (osteopontin, bone sialoprotein I, early T-lymphocyte activation 1) |
| 4 | AW958124 | Hs.142442 | HP1-BP74 |
| 4 | AL137714 | Hs.356298 | hypothetical protein LOC58481 |
| 4 | AA001266 | Hs.133521 | ESTs |
| 4 | AL133100 | Hs.377705 | hypothetical protein FLJ20531 |
| 4 | AA001615 | Hs.84561 | ESTs |
| 4 | AA568515 | Hs.293510 | ESTs |
| 4 | AW079749 | Hs.184719 | ESTs, Weakly similar to ALU1_HUMAN ALU SUBFAMILY J SEQUENCE |
| | | | CONTAMINATION WARNING ENTRY [H. sapiens] |
| 4 | AL045285 | Hs.277401 | bromodomain adjacent to zinc finger domain, 2A |
| 4 | AI740647 | Hs.141012 | ESTs, Weakly similar to ALU1_HUMAN ALU SUBFAMILY J SEQUENCE |
| | | | CONTAMINATION WARNING ENTRY [H. sapiens] |
| 4 | AW976347 | Hs.76966 | ESTs |
| 4 | AI191811 | Hs.54629 | ESTs |

TABLE 4
CLUSTER 1 TOP TARGETS

| Training | | | | |
|---|---|---|---|---|
| Data | | | | |
| Effective | | Exemplar | | |
| Weights | SEQ ID NOs: | Accession | UniGene ID | UniGene Title |
| 1.202 | 8 & 29 | BE262656 | Hs.32603 | hypothetical protein MGC3279 similar to collectins |
| 1.048 | 9, 18 & 30 | AW382884 | Hs.5320 | MGC16824 Esophageal cancer associated protein |
| 0.958 | 10, 11, 31 & 32 | AW847814 | Hs.289005 | Homo sapiens cDNA: FLJ21532 fis, clone COL06049 |
| 0.773 | 12 & 33 | W25945 | Hs.8173 | hypothetical protein FLJ10803 |
| 0.763 | 13, 19 & 34 | AI698091 | Hs.107845 | ESTs |
| 0.666 | | AI205785 | Hs.190153 | Unnamed protein product [H. sapiens] |
| 0.625 | | AL043983 | Hs.125063 | Homo sapiens cDNA FLJ13825 fis, clone THYRO1000558 |
| 0.503 | | AA531129 | Hs.190297 | ESTs |
| 0.492 | | NM_173483 | NA | ESTs |
| 0.352 | | BE327477 | Hs.166941 | ESTs |
| 0.332 | | AI936504 | Hs.2083 | CDC-like kinase 1 |
| 0.031 | | R66282 | Hs.20247 | ESTs, Weakly similar to S65657 alpha-1C-adrenergic |
| | | | | receptor splice form 2 [H. sapiens] |
| 0.030 | | AC007050.25 | NA | ESTs |
| 0.023 | | BE378541 | Hs.279815 | cysteine sulfinic acid decarboxylase-relatedprotein 2 |
| −0.028 | | AA907305 | Hs.36475 | ESTs |
| −0.098 | | AW748336 | Hs.168052 | KIAA0421 protein |
| −0.466 | | AI034357 | Hs.211194 | ESTs, Weakly similar to ALU8_HUMAN ALU |
| | | | | SUBFAMILY SX SEQUENCE CONTAMINATION |
| | | | | WARNING ENTRY [H. sapiens] |
| −0.666 | | AW976570 | Hs.97387 | ESTs |
| −0.996 | 14, 20 & 35 | AW054922 | Hs.53478 | Homo sapiens cDNA FLJ12366 fis, clone MAMMA1002411 |
| −1.065 | 15, 21 & 36 | AA830893 | Hs.119769 | ESTs |

TABLE 5
CLUSTER 4 TOP TARGETS

| Training Data | | | | |
|---|---|---|---|---|
| Effective | SEQ ID | Exemplar | | |
| Weights | NOs: | Accession | UniGene ID | UniGene Title |
| 2.041 | 1 & 22 | AU076643 | Hs.313 | secreted phosphoprotein 1 (osteopontin, bone sialoprotein |
| | | | | I, early T-lymphocyte activation 1) |
| 1.644 | 2 & 23 | AA132637 | Hs.15396 | Homo sapiens, clone IMAGE: 3948909, mRNA, partial |
| | | | | cds |
| 1.244 | 3, 16, & 34 | AW276914 | Hs.423341 | Homo sapiens clone IMAGE: 713177, mRNA sequence |
| 1.171 | 4 & 25 | AL133100 | Hs.377705 | hypothetical protein FLJ20531 - NM_017865 |
| 1.162 | 5, 17 & 26 | AA612710 | Hs.284148 | ESTs |
| 0.896 | 6 & 27 | AL137714 | Hs.356298 | hypothetical protein LOC58481 |
| 0.488 | | AI800041 | Hs.369733 | ESTs |
| 0.437 | | AI982794 | Hs.159473 | ESTs |
| 0.217 | | AL045285 | Hs.277401 | BAZ2A, Bromodomain adjacent to zinc finger domain, |
| | | | | 2A |
| 0.138 | | T64896 | Hs.406798 | Homo sapiens cDNA FLJ11533 fis, clone |
| | | | | HEMBA1002678 |
| 0.040 | | AA425310 | Hs.155766 | ESTs, Weakly similar to A47582 B-cell growth factor |
| | | | | precursor [H. sapiens] |
| −0.056 | | AW976347 | Hs.76966 | ESTs |
| −0.127 | | H74319 | Hs.188620 | ESTs |
| −0.298 | | AW079749 | Hs.184719 | ESTs |
| −0.303 | | AI459519 | Hs.314437 | clone IMAGE: 4607209, mRNA sequence [H. sapiens] |
| −0.319 | | H77824 | NA | ESTs |
| −0.321 | | AA830335 | Hs.105273 | ESTs |
| −0.602 | | W22152 | Hs.282929 | ESTs |
| −0.723 | | R07785 | Hs.429867 | ESTs |
| −1.306 | 7 & 28 | U50535 | Hs.110630 | Human BRCA2 region, mRNA sequence CG006 |

TABLE 6

FULL LENGTH NUCLEIC ACID AND PROTEIN SEQUNCES OF SOME GENES THAT
CHARACTERIZE METASTATIC COLORECTAL CANCER

| | |
|---|---|
| NUCLEIC ACID SEQUENCES | |
| Seq ID NO: 1 | |
| Primekey #: 446619 | |
| Coding sequence: 88..990 | |
| 1 11 21 31 41 51 | |
| \| \| \| \| \| \| | |
| GCAGAGCACA GCATCGTCGG GACCAGACTC GTCTCAGGCC AGTTGCAGCC TTCTCAGCCA | 60 |
| AACGCCGACC AAGGAAAACT CACTACCATG AGAATTGCAG TGATTTGCTT TTGCCTCCTA | 120 |
| GGCATCACCT GTGCCATACC AGTTAAACAG GCTGATTCTG GAAGTTCTGA GGAAAAGCAG | 180 |
| CTTTACAACA AATACCCAGA TGCTGTGGCC ACATGGCTAA ACCCTGACCC ATCTCAGAAG | 240 |
| CAGAATCTCC TAGCCCCACA GACCCTTCCA AGTAAGTCCA ACGAAAGCCA TGACCACATG | 300 |
| GATGATATGG ATGATGAAGA TGATGATGAC CATGTGGACA GCCAGGACTC CATTGACTCG | 360 |
| AACGACTCTG ATGATGTAGA TGACACTGAT GATTCTCACC AGTCTGATGA GTCTCACCAT | 420 |
| TCTGATGAAT CTGATGAACT GGTCACTGAT TTTCCCACGG ACCTGCCAGC AACCGAAGTT | 480 |
| TTCACTCCAG TTGTCCCCAC AGTAGACACA TATGATGGCC GAGGTGATAG TGTGGTTTAT | 540 |
| GGACTGAGGT CAAAATCTAA GAAGTTTCGC AGACCTGACA TCCAGTACCC TGATGCTACA | 600 |
| GACGAGGACA TCACCTCACA CATGGAAAGC GAGGAGTTGA ATGGTGCATA CAAGGCCATC | 660 |
| CCCGTTGCCC AGGACCTGAA CGCGCCTTCT GATTGGGACA GCCGTGGGAA GGACAGTTAT | 720 |
| GAAACGAGTC AGCTGGATGA CCAGAGTGCT GAAACCCACA GCCACAAGCA GTCCAGATTA | 780 |
| TATAAGCGGA AAGCCAATGA TGAGAGCAAT GAGCATTCCG ATGTGATTGA TAGTCAGGAA | 840 |
| CTTTCCAAAG TCAGCCGTGA ATTCCACAGC CATGAATTTC ACAGCCATGA AGATATGCTG | 900 |
| GTTGTAGACC CCAAAAGTAA GGAAGAAGAT AAACACCTGA AATTTCGTAT TTCTCATGAA | 960 |
| TTAGATAGTG CATCTTCTGA GGTCAATTAA AAGGAGAAAA AATACAATTT CTCACTTTGC | 1020 |
| ATTTAGTCAA AAGAAAAAAT GCTTTATAGC AAAATGAAAG AGAACATGAA ATGCTTCTTT | 1080 |
| CTCAGTTTAT TGGTTGAATG TGTATCTATT TGAGTCTGGA AATAACTAAT GTGTTTGATA | 1140 |
| ATTAGTTTAG TTTGTGGCTT CATGGAAACT CCCTGTAAAC TAAAAGCTTC AGGGTTATGT | 1200 |
| CTATGTTCAT TCTATAGAAG AAATGCAAAC TATCACTGTA TTTTAATATT TGTTATTCTC | 1260 |
| TCATGAATAG AAATTTATGT AGAAGCAAAC AAAATACTTT TACCCACTTA AAAAGAGAAT | 1320 |
| ATAACATTTT ATGTCACTAT AATCTTTTGT TTTTTAAGTT AGTGTATATT TTGTTGTGAT | 1380 |
| TATCTTTTTG TGGTGTGAAT AAATCTTTTA TCTTGAATGT AATAAGAATT TGGTGGTGTC | 1440 |
| AATTGCTTAT TTGTTTTCCC ACGGTTGTCC AGCAATTAAT AAAACATAAC CTTTTTTACT | 1500 |
| GCCTAAAAAA AAAAAAAAAA AAAA | 1524 |
| Seq ID NO: 2 | |
| Primekey #: 408199 | |
| Coding sequence: 27..734 | |
| 1 11 21 31 41 51 | |
| \| \| \| \| \| \| | |
| GTGCAAGCAT CTGAAGAGCT GCCGGGATGC AGCAGAGAGG AGCAGCTGGA AGCCGTGGCT | 60 |
| GCGCTCTCTT CCCTCTGCTG GGCGTCCTGT TCTTCCAGGG TGTTTATATC GTCTTTTCCT | 120 |
| TGGAGATTCG TGCAGATGCC CATGTCCGAG GTTATGTTGG AGAAAAGATC AAGTTGAAAT | 180 |
| GCACTTTCAA GTCAACTTCA GATGTCACTG ACAAACTTAC TATAGACTGG ACATATCGCC | 240 |
| CTCCCAGCAG CAGCCACACA GTATCAATAT TTCATTATCA GTCTTTCCAG TACCCAACCA | 300 |
| CAGCAGGCAC ATTTCGGGAT CGGATTTCCT GGGTTGGAAA TGTATACAAA GGGGATGCAT | 360 |
| CTATAAGTAT AAGCAACCCT ACCATAAAGG ACAATGGGAC ATTCAGCTGT GCTGTGAAGA | 420 |
| ATCCCCCAGA TGTGCATCAT AATATTCCCA TGACAGAGCT AACAGTCACA GAAAGGGGTT | 480 |
| TTGGCACCAT GCTTTCCTCT GTGGCCCTTC TTTCCATCCT TGTCTTTGTG CCCTCAGCCG | 540 |
| TGGTGGTTGC TCTGCTGCTG GTGAGAATGG GGAGGAAGGC TGCTGGGCTG AAGAAGAGGA | 600 |
| GCAGGTCTGG CTATAAGAAG TCATCTATTG AGGTTTCCGA TGACACTGAT CAGGAGGAGG | 660 |
| AAGAGGCGTG TATGGCGAGG CTTTGTGTCC GTTGCGCTGA GTGCCTGGAT TCAGACTATG | 720 |
| AAGAGACATA TTGATGAAAG TCTGTATGAC ACAAGAAGAG TCACCTAAAG ACAGGAAACA | 780 |
| TCCCATTCCA CTGGCAGCTA AAGCCTGTCA GAGAAAGTGG AGCTGGCCTG GACCATAGCG | 840 |
| ATGGACAATC CTGGAGATCA TCAGTAAAGA CTTTAGGAAC CACTTATTTA TTGAATAAAT | 900 |
| GTTCTTGTTG TATTTATAAA CTGTTCAGGA ACTCTCATAA GAGACTCATG ACTTCCCCTT | 960 |
| TCAATGAATT ATGCTGTAAT TGAATGAAGA AATTCTTTTC CTGAGCAAAA AGATACTTTT | 1020 |
| TGATTCATCT TTGCTCTGGA ATGTATTACA TGTTTTCTTC CAACTGTTTG AAGGAGAATT | 1080 |
| TTGAATGTTT GCCACACCGC TGATACCCAA ATAATTTTTT AAATGAAGTG GAGCTTGTGG | 1140 |
| CTTCCTGATG TGTCACCAGA CAAAATATTC GCTTGGGATA TGTATTCTTT GTTTTTTGCT | 1200 |
| CCATGTACAC TTTCAGCTGT GAGTTAGTAT AGGGCGTATA CTTACCGGTT TAATGACCTC | 1260 |
| AACCTCAGTT GTGTTTGGAT AACTTAGGGT GTATACCCTT AGTTTCCTTA GAGTTGGTAG | 1320 |
| GATCAAGTCA TTGGTTTGCT TTGACTGGGT TTTTAAAGTA TTAAGTACAG TGTCATCAAT | 1380 |
| TTACAGTTAA GGAAAGGAAT CGTGAAGTAG AAAAATTATT TTCTTTAGTC TTGCTGGTAC | 1440 |
| AATTTGGGCT AAGGAGTCTT TGTTATTTTC TGTCTTGCTT TTTTTTTTTT TTTTTTTTTT | 1500 |
| TTGAGGCAGA GTCTCACTCT GTCGCCAGGC TGGAGTGCAG TGGTGTGATC TTGGCTCACT | 1560 |
| GCAACCTCTG CCTCCTGGGT TCAAGCGATT CTTGTGCCTC AGCCTCTCGA GTAGCTGGGA | 1620 |
| TTACAGGCAT GCGCCACCAC ACCCAGCTAA TTTTTGTGTT TTTAGTAGAG ACGGGGTTTC | 1680 |
| ACCATTTTGG CCAGGATGGT CTCAATCCCC TGACCTCGTG ATCCACCTGC CTCGGCCTCC | 1740 |
| CAAAGTGTTG GGATTACAGG CATGAGCCAC TGTGCTTGGC CTGTTATTTT ATTTTCTTAT | 1800 |
| AACTACAACT TTTCTTCTTG AATTTTCAGG TCAGAGGCAA GAAAAACTCT TTACAGGTTT | 1860 |
| TTAGTGGGGG GCTTATGGAG TATTTCAGGA GTTCTTTGCA AATTAAATCA TCTTTTCACT | 1920 |
| TGTATTGTTT TTCAAAACTT TGTTGATTTC TAAAATGTGC CAACTGTGAG TAAACTATGG | 1980 |
| TATTTGCAAG TGGTTTTTAC ATAATATTTG AGATGAGGAA GTGAGATTGT GCATGACATA | 2040 |
| CTTCTCCTTT GTATTCTCTC AGTGCCTTAC AGCAGGTTAC TCCATTCTGC TATGACAACT | 2100 |
| TGTTTCAAAT GTTAATTTAC ATAGGATTTT TTATAAGCCA TTAAGGCATA TGTATAGTAT | 2160 |
| ATCAGTAAAG ATGGATGGTG CATATATAAA TAGTCTTCTG TAATAGTGAT TGGATTTACT | 2220 |
| TCTCAATTAT GAGAGACAAA AATTATCCCC TCACCTGTCT CTATTCTTTC AACAGGTTGA | 2280 |
| TCCCTTTTCA TGATTTTTCA TTAGGTGGTT CAGGAAGTTT CCATATTACA GCGCTTCAGA | 2340 |
| CTGTATATGT TAGTTTAAAA ATCACTTTTC TCTCTCTCAA CTTCTTTCTT TTTTTTTTGA | 2400 |
| AGACTTAATT TAAAAAATTT GGGTTGTTAG ATCCGTATCA TAGATTTGGC CTAGCCTCTT | 2460 |
| CTGTTAACCT AGTCCACAGA TGAGCGAATC TGGTTAGTTG AAGGACATTG TGATTTGACT | 2520 |
| CTGGTCACGC GAGGAAGTAG AAGGGCAAAG ACAGGACCGG CAGTTTACAT TTCCAGTGGT | 2580 |
| TAAACCTCAC GGTACTTTGG GACTGCTTGT TAACTTTTGT GGTTGTCTGA GGCCAATCTA | 2640 |
| ACGTGACCAT TTCTGACACC TCAACAGAGA GAGGAAAGCA ACTTGAGCAA TGAGAGTAAA | 2700 |
| TAACTTGGGC TCTCAGAGAT TTGAAGATAG AGATCTCATT GTGAGGGGGA CTATTTTGCA | 2760 |
| GGTCCTCATT TCTCCAAGAA AGAGATGGTG TTACAGGAAC CCACTGAAAG CCATATCCCA | 2820 |
| TTAAATGAGG AACTAATTTT GGCTGGGCCT TCTTGTAATG TCCTCGCAGG TGTGTTGTGA | 2880 |
| AGATTAATGC AGGGTAGTAT GTTTGTAGAT TGACACCTAG TCTAAACTTG AGGTAATTGG | 2940 |
| TGCTCTGTGA ATACTCAGTC GTGTTCTTTT ATAGCCTTAA TCATGATTTG AACTAGTCCC | 3000 |
| TTGCTTTTTA AATGACTGAA TGAAGTCCTT CGTGGTAAGG GAGTACGTTG ATAACTTAGT | 3060 |
| TTACTATATG GGTTTGTGGT CGCATCCCAG TCATCAGCTG CTATCATTTT CCTTCTTCAT | 3120 |
| CCCTTATACT GAGATTTGGG TTACAGCTTT TTATTCTTCG AAGGATCACA AAGCAGTGTA | 3180 |
| CAGACACCTG CCTTCTTTAA GGATGAAAGG AAGATAAAGT GGTCTTTTTT TGTTTACTTA | 3240 |
| TTTGTTTCAC CTCTTGTTTG AGTAACTTCT AAGGTGCTAT TCTCTCTCTC TTTTTGCTAC | 3300 |
| CTCATGAGCT CTTGTCACAG CCATGGAAAC CAGCCTCGTT TAGAAAGGGA ACTTAGTTCA | 3360 |
| GAAGGGGTTA AAAGCCTTCC AGAATTTTTC TTTAGCTGCT GAAGTTTTTA CATGTGGTTA | 3420 |
| CATGACTTTA AGTTTTATGC ATTACGCTCT TAATTCTATT ACAAAATGTG GACTCACCAA | 3480 |
| TTGCTTTGTG TTTTCCATGT GACCTGTTAC TTCAGGCTAC TTGGGGAACA TCTTAGTCCT | 3540 |
| CTGTAGCTCC TGAACCCAGC ACTGGTGCTT CAAGAGAGAA GGTAGCACGT CTTTGTTCAA | 3600 |
| AACAAAACAA AACGACACTT CTGGAGGCCA CATCCTGAAT ATGAATGTTC TACTAAGTCA | 3660 |
| CTCAGTTATG GTTCTAAAGG GAAACTGTAA GAAGACCCAC AAGGAGTGGA CCAAGACTAT | 3720 |
| TATTTAATTG CACAACTTGA AACTTTGCTG CCAGAAGAGG CAGCTCCATT CCTTTGACTC | 3780 |
| CAGTGTTGGG CTGTTAACTG CTGCACCTCA TTGCCTTTTT TTGTTTTTGT TTTTGTTTTG | 3840 |
| TAGGAGGGTA GGCACTGTTG GGCCATATGC ACAAATATTG TAACTCTTGG TATCTTTACT | 3900 |
| GCATCATAGT CAATAAACTT CTTTGTACCC TT | 3932 |
| Seq ID NO: 3 | |
| Primekey #: 421221 | |
| Coding sequence: 782..1885 | |
| 1 11 21 31 41 51 | |
| \| \| \| \| \| \| | |
| TGAAGGTAAA ATTTTCCAGA TACGGCAGAC GGCTTTCAGA GTACAATAAA CAGGGAATGA | 60 |
| GAACTATTTA CATGGAAGTT TCTTTCTCAT GATGCGGTGG AGAAGCCTCG GCCACTTGGT | 120 |
| TCTGCCAGAT GTTCCTGGGG TTACTGTAAA TGGGAAGGAC AGGCAGAGCT AAACAAGGTT | 180 |
| TATCATTTAA AAGTGCCTGT GTGAAGTCAC TTTTGCTGGA AAACTGCAGC TTGGGAGCTT | 240 |
| TCTTTGTATT CACATCCCAC TCTTCTGTCA AGTACACTTT ACCCTGACCT TATGAGTGGA | 300 |
| TGAAGATACC TCAGTTGTCT GACTTTGCCA ATTGCTTAAT TTCAGAATTT AAAAAGGGGA | 360 |
| AAGAAAAACA TCCTGCTAAA ATATGAACAT CTGAGTGTCT TATTTTCCAA CATCGTCAAT | 420 |
| AGCTGTGAGC GTCAGCATTA AATATTCTCC CAAGGAGTGC CATGATATTG AAGTCACTTT | 480 |
| ATTAATAACA GCTGTATCTG CAAAACAGTC AAGAGACTCG GACGTTGAAA GCCAGAGATG | 540 |
| ACACTGAGCA TGCTTTTATT GCGGCCTACC ATCTTTAAGT GGGACATATT GATTGATGAG | 600 |
| TGATTGCCTG TCCATACACT CTCTCATCAT CCTGTTCCTT GGATTGGACT TCACTAAGCA | 660 |
| ATTTATCACT CACCTTCAGA CTTACATGTG GGAGTTTTCA CAACAGTAGT TTTGGAATCA | 720 |
| TTAGAACTTG GATTGATTTC ATCATTTAAC AGAAACAAAC AGCCCAAATT ACTTTATCAC | 780 |
| CATGGCTTTG AACGTTGCCC CAGTCAGAGA TACAAAATGG CTGACATTAG AAGTCTGCAG | 840 |
| ACAGTTTCAA AGAGGAACAT GCTCACGCTC TGATGAAGAA TGCAAATTTG CTCATCCCCC | 900 |
| CAAAAGTTGT CAGGTTGAAA ATGGAAGAGT AATTGCCTGC TTTGATTCCC TAAAGGGCCG | 960 |
| TTGTTCGAGA GAGAACTGCA AGTATCTTCA CCCTCCGACA CACTTAAAAA CTCAACTAGA | 1020 |
| AATTAATGGA AGGAACAATT TGATTCAGCA AAAAACTGCA GCAGCAATGC TTGCCCAGCA | 1080 |
| GATGCAATTT ATGTTTCCAG GAACACCACT TCATCCAGTG CCCACTTTCC CTGTAGGTCC | 1140 |
| CGCGATAGGG ACAAATACGG CTATTAGCTT TGCTCCTTAC CTAGCACCTG TAACCCCTGG | 1200 |
| AGTTGGGTTG GTCCCAACGG AAATTCTGCC CACCACGCCT GTTATTGTTC CCGGAAGTCC | 1260 |
| ACCGGTCACT GTCCCGGGCT CAACTGCAAC TCAGAAACTT CTCAGGACTG ACAAACTGGA | 1320 |
| GGTATGCAGG GAGTTCCAGC GAGGAAACTG TGCCCGGGGA GAGACCGACT GCCGCTTTGC | 1380 |
| ACACCCCGCA GACAGCACCA TGATCGACAC AAGTGACAAC ACCGTAACCG TTTGTATGGA | 1440 |
| TTACATAAAG GGGCGTTGCA TGAGGGAGAA ATGCAAATAT TTTCACCCTC CTGCACACTT | 1500 |
| GCAGGCCAAA ATCAAAGCTG CGCAGCACCA AGCCAACCAA GCTGCGGTGG CCGCCCAGGC | 1560 |
| AGCCGCGGCC GCGGCCACAG TCATGGCCTT TCCCCCTGGT GCTCTTCATC CTTTACCAAA | 1620 |
| GAGACAAGCA CTTGAAAAAA GCAATGGTAC CAGCGCGGTC TTTAACCCCA GCGTCTTGCA | 1680 |
| CTACCAGCAG GCTCTCACCA GCGCACAGTT GCAGCAACAC GCCGCGTTCA TTCCAACAGG | 1740 |
| GTCAGTTTTG TGCATGACAC CCGCTACCAG TATTGTACCC ATGATGCACA GCGCTACGTC | 1800 |
| CGCCACTGTC TCTGCAGCAA CAACTCCTGC AACAAGTGTC CCCTTCGCAG CAACAGCCAC | 1860 |
| AGCCAATCAG ATAATTCTGA AATAATCAGC AGAAACGGAA TGGAATGCCA AGAATCTGCA | 1920 |
| TTGAGAATAA CTAAACATTG TTACTGTACA TACTATCCTG TTTCCTCCTC AATAGAATTG | 1980 |
| CCACAAACTG CATGCTAAAT AAAGATGTAG TTCTTCTGGA CAGACCACAA CTCTAAGAAG | 2040 |
| CTAGTGCTGC TATCTCATAT ATGAGTATTA AATATGGTAT GCTTAGTATA TTCCAACCTA | 2100 |
| AGATAGTTAA CTACCTGAGA CCAGCTGTGA TGTTTAAAGA CATAAAGGAT AAAGTTTACT | 2160 |
| TTTAAAGGGT TTCTAAACAT AGTTTCTGTC CTAGGAATAT TGTCTTATCT CCATAACTAT | 2220 |
| AGCTGATGCA GAAAGTCCAG CCAGTTTACT CATTTCGATT CAGAATATTT CAAATTTAGC | 2280 |
| AATAAACAAT TAGCATTAGT TAAAAAAGAA ACATATTCCA AGGGCAGGTT CGATTCTAGC | 2340 |
| TCTAATTACT GTCATGTCAT TTACCCACTG GATCAAAGGG TATGTTTCAC TTCTTGACAA | 2400 |
| TATAAATGCT GCAGCAAAGA TGAGAGGTGA AGTAAAACCG ATACCTGTCC TGCAGGTCTA | 2460 |
| AAATTTGAAT GGAAATTCAA GCACAAGTAC TGGGGACACA TCAAAGTGTG GTGTTTGGTT | 2520 |
| TGCCTGGAGA TGCCACGTTG AATCATGTGA TTCTAGATTA ACATTAAATA GATTGAAAAA | 2580 |
| GAAACTTTGC ACGGTATGAG CTTCATACCC CACCAAACAA AGTCTTGAAG GTATTATTTT | 2640 |
| ACAAGTATAT TTTTAAAGTT GTTTTATAAG AGAGACTTTG TAGAAGTGCC TAGATTTTGC | 2700 |
| CAGACTTCAT CCAGCTTGAC AAGATTGAGA GGCCCATGCC AACAGTCTAA TCTAAGAGAT | 2760 |
| TAGTCTTTCA AACTCACCAT CCAGTTGCCT GTTACAGAAT AACTCTTCTT AACTAAAAAC | 2820 |
| CTAGTCAAAC AAGGAAGCTG TAGGTGAGGA GATCTGTATA ATATTCTAAT TTAAGTAAGT | 2880 |
| TTGAGTTTAG TCACTGCAAA TTTGACTGTG ACTTTAATCT AAATTACTAT GTAAACAAAA | 2940 |
| AGTAGATAGT TTCACTTTTT AAAAAATCCA TTACTGTTTT GCATTTCAAA AGTTGGATTA | 3000 |
| AAGGGTTGTA ACTGACTACA GCATGGAAAA AAATAGTTCT TTTAATTCTT TCACCTTAAA | 3060 |
| GCATATTTTA TGTCTCAAAA GTATAAAAAA CTTTAATACA AGTACATACA TATTATATAT | 3120 |
| ACACATACAT ATATATACTA TATATGGATG AAACATATTT TAATGTTGTT TACTTTTTTA | 3180 |
| AATACTTGGT TGATCTTCAA GGTAATAGCG ATACAATTAA ATTTTGTTCA GAAAGTTTGT | 3240 |
| TTTAAAGTTT ATTTTAAGCA CTATCGTACC AAATATTTCA TATTTCACAT TTTATATGTT | 3300 |
| GCACATAGCC TATACAGTAC CTACATAGTT TTTAAATTAT TGTTTAAAAA ACAAAACAGC | 3360 |
| TGTTATAAAT GAATATTATG TGTAATTGTT TCAAACATCC ATTTTCTTTG TGAACATATT | 3420 |
| AGTGATTGAA GTATTTTGAC TTTTGAGATT GAATGTAAAA TATTTTAAAT TTGGGATCAT | 3480 |
| CGCCTGTTCT GAAAACTAGA TGCACCAACC GTATCATTAT TTGTTTGAGG AAAAAAAGAA | 3540 |
| ATCTGCATTT TAATTCATGT TGGTCAAAGT CGAATTACTA TCTATTTATC TTATATCGTA | 3600 |
| GATCTGATAA CCCTATCTAA AAGAAAGTCA CACGCTAAAT GTATTCTTAC ATAGTGCTTG | 3660 |
| TATCGTTGCA TTTGTTTTAA TTTGTGGAAA AGTATTGTAT CTAACTTGTA TTACTTTGGT | 3720 |
| AGTTTCATCT TTATGTATTA TTGATATTTG TAATTTTCTC AACTATAACA ATGTAGTTAC | 3780 |
| GCTACAACTT GCCTAAAACA TTCAAACTTG TTTTCTTTTT TCTGTTTTTT TCTTTGTTAA | 3840 |
| TTCATTTAAA CTCATTGAAA ACATAGTATA CATTACTAAA AGGTAAATTA TGGGAATCAC | 3900 |
| TGAAATATTT TTGTAGATTA ATTGTTGTAA CATTGTCTTT CTTTTTTTTC TTTTGTTTCA | 3960 |
| TGATTTTGAT TTTTAAAATT ATTAGCACAC AACTATTTTC AGCCCTTTAA TAATGGAGCA | 4020 |
| TCAAAAACAT CACCTGTAAC CCCAAGCAAA TATAGAAGAC TGTATTTTTT ACTATGATAT | 4080 |
| CCATTTTCCA GAATTGTGAT TACAATATGC AAAGAGTCAT AAATATGCCA TTTACAATAA | 4140 |
| GGAGGAGGCA AGGCAAATGC ATAGATGTAC AAATATATGT ACAACAGATT TTGCTTTTTA | 4200 |
| TTTATTTATA ATGTAATTTT ATAGAATAAT TCTGGGATTT GAGAGGATCT AAAACTATTT | 4260 |
| TTCTGTATAA ATATTATTTG CCAAAAGTTT GTTTATATTC AGAAGTCTGA CTATGATGAA | 4320 |
| TAAATCTTAA ATGCTTTGTT TAATTAAAAA ACAAAAATCA CCAATATCCA AGACATGAAG | 4380 |
| ATATCAGTTC AACAAATACT GTAGTTAAGA GACTAACTCT CCACTTGTAT GGGAACTACA | 4440 |
| TTTCACTCTT GGTTTTCAGG ATATAACAGC ACTTCACCGA AATATTCTTT CAGCCATACC | 4500 |
| ACTGGTAACA TTTCTACTAA ATCTTTCTGT AACACTTAAA GAATTCCCTC ATTCATTACC | 4560 |
| TTACAGTGTA AACAGGAGTC TAATTTGTAT CAATACTATG TTTTGGTTGT AATATTCAGT | 4620 |
| TCACTCACCC AATGTACAAC CAATGAAATA AAAGAAGCAT TTAAA | 4665 |
| Seq ID NO: 4 | |
| Primekey #: 449491 | |
| Coding sequence: 168..1727 | |
| 1 11 21 31 41 51 | |
| \| \| \| \| \| \| | |
| AGCAGCCGAC GCCGAGAGGC ACCGTTTCTT CTTAAAAGAG AAACGCTGCG CGCGCGAGGT | 60 |
| GGGCCCCTGT CTTCCAGCAG CTCCGGGCCT GCTCGCTAGG CCCGGGAGGC GCAGGCGCAG | 120 |
| GCGCAGTGGG GGTGAGGGCG CGTGGGGGCG CACAGCCTCT GGTGCACATG GCTTCCTCCC | 180 |
| CGGCGGTGGA CGTGTCCTGC AGGCGGCGGG AGAAGCGGCG GCAGCTGGAC GCGCGCCGCA | 240 |
| GCAAGTGCCG CATCCGCCTG GGCGGCCACA TGGAGCAGTG GTGCCTCCTC AAGGAGCGGC | 300 |
| TGGGCTTCTC CCTGCACTCG CAGCTCGCCA AGTTCCTGTT GGACCGGTAC ACTTCTTCAG | 360 |
| GCTGTGTCCT CTGTGCAGGT CCTGAGCCTT TGCCTCCAAA AGGTCTGCAG TATCTGGTGC | 420 |
| TCTTGTCTCA TGCCCACAGC CGAGAGTGCA GCCTGGTGCC CGGGCTTCGG GGGCCTGGCG | 480 |
| GCCAAGATGG GGGGCTTGTG TGGGAGTGCT CAGCAGGCCA TACCTTCTCC TGGGGACCCT | 540 |
| CTTTGAGCCC TACACCTTCA GAGGCACCCA AGCCAGCCTC CCTTCCACAT ACTACTCGGA | 600 |
| GAAGTTGGTG TTCCGAGGCC ACGAGTGGGC AGGAGCTTGC AGATTTGGAA TCTGAGCATG | 660 |
| ATGAGAGGAC TCAAGAGGCC AGGTTGCCCA GGAGGGTGGG ACCCCCACCA GAGACCTTCC | 720 |
| CACCTCCAGG AGAGGAAGAG GGTGAGGAAG AAGAGGACAA TGATGAGGAT GAAGAGGAGA | 780 |
| TGCTCAGTGA TGCCAGCTTA TGGACCTACA GCTCCTCCCC AGATGATAGT GAGCCTGATG | 840 |
| CCCCCAGACT ACTGCCTTCC CCTGTCACCT GCACACCTAA AGAGGGGGAG ACACCACCAG | 900 |
| CCCCTGCAGC ACTCTCCAGT CCTCTTGCTG TGCCGGCCTT GTCAGCATCC TCATTGAGTT | 960 |
| CCAGAGCTCC TCCACCTGCA GAAGTCAGGG TGCAGCCACA GCTCAGCAGG ACCCCTCAAG | 1020 |
| CGGCCCAGCA GACTGAGGCC CTGGCCAGCA CTGGGAGTCA GGCCCAGTCT GCTCCAACCC | 1080 |
| CGGCCTGGGA TGAGGACACT GCACAAATTG GCCCCAAGAG AATTAGGAAA GCTGCCAAAA | 1140 |
| GAGAGCTGAT GCCTTGTGAC TTCCCTGGCT GTGGAAGGAT CTTCTCCAAC CGGCAGTATT | 1200 |
| TGAATCACCA CAAAAAGTAC CAGCACATCC ACCAGAAGTC TTTCTCCTGC CCAGAGCCAG | 1260 |
| CCTGTGGGAA GTCTTTCAAC TTTAAGAAAC ACCTGAAGGA GCACATGAAG CTGCACAGTG | 1320 |
| ACACCCGGGA CTACATCTGT GAGTTCTGCG CCCGGTCTTT CCGCACTAGC AGCAACCTTG | 1380 |
| TCATCCACAG ACGTATCCAC ACTGGAGAAA AACCCCTGCA GTGTGAGATA TGCGGGTTTA | 1440 |
| CCTGCCGCCA GAAGGCTTCC CTGAACTGGC ACCAGCGCAA GCATGCAGAG ACGGTGGCTG | 1500 |
| CCTTGCGCTT CCCCTGTGAA TTCTGCGGCA AGCGCTTTGA GAAGCCAGAC AGTGTTGCAG | 1560 |
| CCCACCGTAG CAAAAGTCAC CCAGCCCTGC TTCTAGCCCC TCAAGAGTCA CCCAGTGGTC | 1620 |
| CCCTAGAGCC CTGTCCCAGC ATCTCTGCCC CTGGGCCTCT GGGATCCAGC GAGGGGTCCA | 1680 |
| GGCCCTCTGC ATCTCCTCAG GCTCCAACCC TGCTTCCTCA GCAATGAGCT CTCCTCCAGC | 1740 |
| TTTGGCTTTG GGAAGCCAGA CTCCAGGGAC TGAAAAGGAG CAACAAGGAG AGGGTCTGCT | 1800 |
| TGAGAAATGC CAGATGCTTG GTCCCCAGGA ACTAAGGCGA CAGAGTGCAG GGTGGGGGCA | 1860 |
| AGACTGGGCT GTAGGGGAGC TGGACTACTT TAGTCTTCCT AAAGGACAAA ATAAACAGTA | 1920 |
| TTTTATGCAG GAAAAAAAAA AAAAAAAAAA AAAAAAAAAA AAAAAAAAAA AAAAAAAAAA | 1980 |
| AAAAAA | 1986 |
| Seq ID NO: 5 | |
| Primekey #: 429766 | |
| Coding sequence: 483..1145 | |
| 1 11 21 31 41 51 | |
| \| \| \| \| \| \| | |
| CGGACGCGTG GGCTGAGGCG GCGCTGTGTG TGTGAAGCGT ACCTAGGGCG GGAGGCGACA | 60 |
| TGGAGACAGG GGCGGCCGAG CTGTATGACC AGGCCCTTTT GGGCATCCTG CAGCACGTGG | 120 |
| GCAACGTCCA GGATTTCCTG CGCGTTCTCT TTGGCTTCCT CTACCGCAAG ACAGACTTCT | 180 |
| ATCGCTTGCT GCGCCACCCA TCGGACCGCA TGGGCTTCCC GCCCGGGGCC GCGCAGGCCT | 240 |
| TGGTGCTGCA GGTATTCAAA ACCTTTGACC ACATGGCCCG TCAGGATGAT GAGAAGAGAA | 300 |
| GGCAGGAACT TGAAGAGAAA ATCAGAAGAA AGGAAGAGGA AGAGGCCAAG ACTGTGTCAG | 360 |
| CTGCTGCAGC TGAGAAGGAG CCAGTCCCAG TTCCAGTCCA GGAAATAGAG ATTGACTCCA | 420 |
| CCACAGAATT GGATGGGCAT CAGGAAGTAG AGAAAGTGCA GCCTCCAGGC CCTGTGAAGG | 480 |
| AAATGGCCCA TGGTTCACAG GAGGCAGAAG CTCCAGGAGC AGTTGCTGGT GCTGCTGAAG | 540 |
| TCCCTAGGGA ACCACCAATT CTTCCCAGGA TTCAGGAGCA GTTCCAGAAA AATCCCGACA | 600 |
| GTTACAATGG TGCTGTCCGA GAGAACTACA CCTGGTCACA GGACTATACT GACCTGGAGG | 660 |
| TCAGGGTGCC AGTACCCAAG CACGTGGTGA AGGGAAAGCA GGTCTCAGTG GCCCTTAGCA | 720 |
| GCAGCTCCAT TCGTGTGGCC ATGCTGGAGG AAAATGGGGA GCGCGTCCTC ATGGAAGGGA | 780 |
| AGCTCACCCA CAAGATCAAC ACTGAGAGTT CTCTCTGGAG TCTCGAGCCC GGGAAGTGCG | 840 |
| TTTTGGTGAA CCTGAGCAAG GTGGGCGAGT ATTGGTGGAA CGCCATCCTG GAGGGAGAAG | 900 |
| AGCCCATCGA CATTGACAAG ATCAACAAGG AGCGCTCCAT GGCCACCGTG GATGAGGAGG | 960 |
| AACAGGCGGT GTTGGACAGG CTTACCTTTG ACTACCACCA GAAGCTGCAG GGCAAGCCAC | 1020 |
| AGAGCCATGA GCTGAAAGTC CATGAGATGC TGAAGAAGGG GTGGGATGCT GAAGGTTCTC | 1080 |
| CCTTCCGAGG CCAGCGATTC GACCCTGCCA TGTTCAACAT CTCCCCGGGG GCTGTGCAGT | 1140 |
| TTTAATGACC AGAAGGAAAG GAAACCCTCG CCGGTGGGGA GGCAGAGCCT TATCCTCGGC | 1200 |
| TGCCCTTCTT GGCTCCCTGC ATTCCAGGGA CTTGCTCGTC TTGTTTACCC CTAGCCATCC | 1260 |
| TTTCTTTCAA GGGTGAACCA GGCCTTCCAC CCTGACCTTG CATCTCCAGA CTGTTCCAGA | 1320 |
| GAAGGTGCGG GGCCAGCTGC TATGTGGTGG CCGCTGTGGC TGACACTGAG TGAAGGTGTT | 1380 |
| TGAAATGCAG GAGAGGATAT CCCAGCAAAT TGGGATCACA TGCTTTTGTC TCCACAGCAA | 1440 |
| CCAGCCACTG CAGGCAGCAT GTCTTTCCTC CCCTGCTCTC TGCTTGCTGT TGTTTTGACG | 1500 |
| CTATTCTGCT TGCATGTCTT CTGGTTGGGA TGTGGAGTTG TTGCTGGACT CTCAGGCGAA | 1560 |
| GCTGAAGTCA TTGAAGTGTG TGAAGCTCTG TGCTTGCATG AGGGCAAGCA AGGAATGGCT | 1620 |
| GTGCCTGAGG CTGCTCTGGG AAACTCCTTG CCCCTTGACC TCTTTTGAGA GCATTCACGT | 1680 |
| GGTCTTCTTG CTCATCCCCT TATAAATGTG CTTTGCCTGC CTCAGCCTCA TGGTCAGAGC | 1740 |
| AGTGGAGACT GGAGCCCTGT TTGCACGTTC TAGTTGTTCG GAGAAAGCCT AGGTTCTGGG | 1800 |
| CTCAGGTCCA GATGCAGCGG GGATTCTGTT CTCTGACTGT GGCGACCTTG CTTTGGTTCT | 1860 |
| TGTTGAAGTG AACCAAGCCC GGCCACCACG CATGGCATGC TGTGCTTGGC TCCCCATAAG | 1920 |
| ACGTCCTCTT TGGGTGCACG GTGTCAAAGT GTGGGCAGGA GTGGAGAGCT GGTGCCCTCA | 1980 |
| GGAGGAGACC ACAGCATGTC CATCAGCTCA GCAGAGCTCG ACAGCCACAA GTCCTGAGAA | 2040 |
| GCTTTGACCT TGAAGGGCTT CTGGGAGAGG AGGAATTTCT GCATGGGGCG TGAAGGCACA | 2100 |
| CTGTCCCACC ACAACTGAAC CAGAAGAGAG TGAAGACTCC CCTCTTCCCA TCCTCTGTGC | 2160 |
| CAGGTGCCAG ACTGTGCTCC TTGGAACTTA TGGCCCAATC TTACCTGTTC TCCAGGGACT | 2220 |
| GGTCACTGCC TCAGGACCCC CAAGCCTATG CCCTGAGCCA TGGCTGCTGA CTGACTCCAG | 2280 |
| CCAAGGTGCA AAGACGAGAT TATGAGACAG GTCCTCAGGC CTGTGTTCCA AGTACTCACA | 2340 |
| GGGGCTCTGG GTGCCCATCG CCGGGAGTAT GGTTCAGCTG CCACCGGCAC TGTCCATTTG | 2400 |
| CCTGTCTGTC AAGCTCAGAG CATGGATAAG CCACACAGCA GGGCAGTGCA CCCTGGCACC | 2460 |
| ATGCACGGCC AGCAAGAATC AAGGCCCGCA GATGCTAAGA GGGCCTATTG TCAGGGGAAG | 2520 |
| GTCCCCGCTC CTGCACACTC TCTATGGATA CTTGGGTTGT GGGGGCTCTC TTGGAGAGTA | 2580 |
| AGTTTGTGGT TTGTTTCTGG TTTACAGTGG TGGCTGACAC CCCTTGTAAG AAAGCATTCC | 2640 |
| TGGGAAGTCT TCTGTGGGTC CAAACATGTT GCTCCGATCA TCACAGGAGA GCAAAAGGCC | 2700 |
| CTAGATACCC CCTTTGGAAT GTGAGAGTCT TGTTGTCTGA TATTTGCCAC TGAGCTGGTG | 2760 |
| AAGCCCCTCT AAAGAGATCT CGACCCTGGG GAGCAGAATT CTTGTCATCT ATGAGGGGTC | 2820 |
| CTGAGAAAGA CTTGTCATTT TTTTTCCTGG AGTTCTTCCC ATTGAGGTCC TAGGATTTGC | 2880 |
| ACACCACTGT CCCACAAGAG CTTTCCTGCC TAATGAAAGG AGGTCTTGTG GTGTGTGTCT | 2940 |
| CCTCTCTTCT CTATAGTTCC CGAGTTGGCC CCCATTGCAG CCCCCACCCT GTGGGTAGTC | 3000 |
| TTCCAGAAGT GATGCAGTGG TGTGAGATGC CCTGCACCTT GTTATTTGGG AGACTTTGAG | 3060 |
| AGTCATTCAC TTCCATGGTG ACTAGTGTTT GTTTTGCCTG ATTTTATATT CTGTGTTGCA | 3120 |
| TTTCTCCCCA CTCCCTGCCC TGCTTTAATA AACAGCAAAC CAATATCTAG GAAGAATGAC | 3180 |
| TGAGGGATAG TATTGGGTAT TGGCCCCATG GCAGGAACAG CCACTTGCAT CTGGTCCCGG | 3240 |
| TGCCACACTG CGGTGCTTGG TGTGGTTGTG GAGCCTGTCC CTGCGCGCCT TGCTCCCGTT | 3300 |
| GAGCCACGCT GTCTGGTGGG TGATTCTCTG CCCTGAGCCA CCACCCTGGA CTGGCCCAGT | 3360 |
| CTCCAGAGCT GGCACACCCT GCCTGTTTTC TCTTTTTAGA CACAACAGCC GCAGTTTGGC | 3420 |
| CAGCCACTAA GTCCCACCAG CTGAGGTCCG AGGAAAGCGG GGTGACTCAT TTCCCTTGTC | 3480 |
| CAGGGCCCGA GGAGAGTGAG GTGTCCAGCC TGCAAAGCTA TTCCAGCTCC TTGGTGTTGG | 3540 |
| TTTGCAATAA ATTGGTATTT AAGCAAAAAA AAAAAAAAAA AAAA | 3584 |
| Seq ID NO: 6 | |
| Primekey #: 448518 | |
| Coding sequence: 1424..1897 | |
| 1 11 21 31 41 51 | |
| \| \| \| \| \| \| | |
| CGTGATCATG AGGGGTTGTG AAGTGCTTGC CCCATCAGTA GCCATGTGTG CATGTGTAAA | 60 |
| TACCATCCTC TGTGTGCCCT GGAGGCTGTC CTTCAGATAG CATGTACAGG TGGCAGCATA | 120 |
| GGGCCTGTCC CTACTGAGAG TGCAGGGAAC TCAGCACCGT CAACTCCTCG ACCCTGCAGG | 180 |
| TCAGATTATC CTTGTAGAGG CCCCCTGGAT GGCACCAAGA TCGGCCCTGG CAAGTAGGTG | 240 |
| ACCCTGACTT CAGAGCCCTT GCCTGAGGGC CTGGCCTGGC AGCTCTGCTG TTAGAAGCAG | 300 |
| GAGGTGTGCA GAGGGTGGGG AGCAGCCCAG CCTCTGTGAT CTTCTCCATG GCAGGATCTC | 360 |
| CCAGCAGGTA GAGCAGAGCC GGAGCCAGGT GCAGGCCATT GGAGAGAAGG TCTCCTTGGC | 420 |
| CCAGGCCAAG ATTGAGAAGA TCAAGGGCAG CAAGAAGGCC ATCAAGGTAG TCCCCATACC | 480 |
| CCTGTGTCCT GAGGCTACTG GGCAGTCCCT CCATTTCCCC GTGCCTCTGA GGCTGCCCAG | 540 |
| TCTCTGCCCT GCTGCCCACC TGTACCTTGA GCTTTCTTCT CGCCCAGGCT TCCAACTCCA | 600 |
| CCCTCTCCTG CCAAGCAATC CTAGCCCTCT GAGCCTCTTG GGGCCCCCTC AGACTTGTCC | 660 |
| CTGTGTCCAC AGGTGTTCTC CAGTGCCAAG TACCCTGCTC CAGGGCGCCT GCAGGAATAT | 720 |
| GGCTCCATCT TCACGGGCGC CCAGGACCCT GGCCTGCAGA GACGCCCCCG CCACAGGATC | 780 |
| CAGAGCAAGC ACCGCCCCCT GGACGAGCGG GCCCTGCAGG TCTGCTGGCC GCGCATATAG | 840 |
| CCTGTCACAC ACCAGGAGGA CTGGATACTG GGGAGGAGCC GGGGCCACCA TAGGGTTCTG | 900 |
| TCCCCCAGAG GAGGCTGACT GGGATGGGAT GGCAGCTGAT TAGGCCCAGC ACCAAATATT | 960 |
| CACCATCCCT TGGCCATCCT GGCCCTCTCA GGAGAAGCTG AAGGACTTTC CTGTGTGCGT | 1020 |
| GAGCACCAAG CCGGAGCCCG AGGACGATGC AGAAGAGGGA CTTGGGGGTC TTCCCAGCAA | 1080 |
| CATCAGCTCT GTCAGCTCCT TGCTGCTCTT CAACACCACC GAGAACCTGT ATGGCCAGAG | 1140 |
| GGCAGGGCCG AGGGGTGTGG GCGGGAGGCC CGGCCTGGCT TAGTGGGGAC CCAGGGCATC | 1200 |
| AGACACAGGT ACAGCACATA GGCCAGGAGC CAGGGGGTGA CGGGTGGCTC GGCTCGGGAG | 1260 |
| GCCTGGGACC CCACAGTGCA CGCTGTGCCC CTGATGATGT GGGAGAGGAA CATGGGCTCA | 1320 |
| GGACAGCGGG TGTCAGCTTG CCTGACCCCC ATGTCGCCTC TGTAGGTAGA AGAAGTATGT | 1380 |
| CTTCCTGGAC CCCCTGGCTG GTGCTGTAAC AAAGACCCAT GTGATGCTGG GGGCAGAGAC | 1440 |
| AGAGGAGAAG CTGTTTGATG CCCCCTTGTC CATCAGCAAG AGAGAGCAGC TGGAACAGCA | 1500 |
| GGTGGGAGGG GTGGGACAGA GGTGGAGACA GGTGCAGTGG CCCAGGGCCT TGCCAGAGCT | 1560 |
| CCTCTCCAGT CAAGGCTGTT GGGCCCCTTA TTCCACCCAT GGGAGGTGCA CACAAGGTCT | 1620 |
| TGTTGGCTGC CCCTGCAGGT CCCTGTCACC TCTCACATGT CCCTGCCTAA TCTTGCAGGT | 1680 |
| CCCAGAGAAC TACTTCTATG TGCCAGACCT GGGCCAGGTG CCTGAGATTG ATGTTCCATC | 1740 |
| CTACCTGCCT GACCTGCCCG GCATTGCCAA CGACCTCATG TACATTGCCG ACCTGGGCCC | 1800 |
| CGGCATTGCC CCCTCTGCCC CTGGCACCAT TCCAGAACTG CCCACCTTCC ACACTGAGGT | 1860 |
| AGCCGAGCCT CTCAAGACCT ACAAGATGGG GTACTAACAC CACCCCCACC GCCCCCACCA | 1920 |
| CCACCCCCAG CTCCTGAGGT GCTGGCCAGT GCACCCCCAC TCCCACCCTC AACCGCGGCC | 1980 |
| CCTGTAGGCC AAGGCGCCAG GCAGGACGAC AGCAGCAGCA GCGCGTCTCC TTCAGGTGGG | 2040 |
| AGCAGCTCTT TGAGGCCACC TGATTTCTGG CGTGCTCAGT GCACTCGGGT GGATTTTCTG | 2100 |
| TGGGTTTGTT AAGTGGTCAG AAATTCTCAA TTTTTTGAAT AGTTTCCATT TCAAATATCT | 2160 |
| TGTTCTACTT GGTTCATAAA ATAGTGGTTT TCAAACTGTA GAGCTCTGGA CTTCTCACTT | 2220 |
| CTAGGGCAGA GGGAGCCTGA ACAAGTGAGG CTCTGGGTTC CCCATTCCTA ATTAAACCAA | 2280 |
| TGGAAAGAAG GGGTCTAATA ACAAACTACA GCAACACATT TTTCATTTCA GCTTCACTGC | 2340 |
| TGTGTCTCCC AGTGTAACCC TAGCATCCAG AAGTGGCACA AAACCCCTCT GCTGGCTCGT | 2400 |
| GTGTGCAACT GAGACTGTCA GAGCATGGCT AGCTCAGGGG TCCAGCTCTG CAGGGTGGGG | 2460 |
| GCTAGAGAGG AAGCAGGGAG TATCTGCACA CAGGATGCCC GCGCTCAGGT GGTTGCAGAA | 2520 |
| GTCAGTGCCC AGGCCCCCAC ACACAGTCTC CAAAGGTCCG GCCTCCCCAG CGCAGGGCTC | 2580 |
| CTCGTTTGAG GGGAGGTGAC TTCCCTCCCA GCAGGCTCTT GGACACAGTA AGCTTCCCCA | 2640 |
| GCCCTGCCTG AGCAGCCTTT CCTCCTTGCC CTGTTCCCCA CCTCCCGGCT CCAGTCCAGG | 2700 |
| GAGCTCCCAG GGAAGTGGTT GACCCCTCCG GTGGCTGGCC ACTCTGCTAG AGTCCATCCG | 2760 |
| CCAAGCTGGG GGCATCGGCA AGGCCAAGCT GCGCAGCATG AAGGAGCGAA AGCTGGAGAA | 2820 |
| GCAGCAGCAG AAGGAGCAGG AGCAAGGTGA GCGGGCCCTG GAGCTTGCAG TCGGAGGGCC | 2880 |
| TTGGGCAAGA TCGCCTCCTC CCCTCCAGCC CTGAGTCCAC CGGGTGCTTT CTGCCCACCC | 2940 |
| CCTGCTCTTG CCAGCTGGCC CCTGCTTCCC CTAGGGCACA TGCTGGAAGC CCTGGGCCGC | 3000 |
| CACCAGAGGT CCTCAGCCCT CCTGCCTGGG CTATGGCTCC TTCCTGGTTT GGGAGCCATA | 3060 |
| GTGGAGCTTT CCTCTCTAAG CTCACCCAGC TCAAACTGAC AGGAGAATCT TCTTCGACTG | 3120 |
| CCAAGAGCGG TCCAAGGCAA TGGTCAGCCA CTGCAGCCTC CTGAGATATT TTTAGAGACT | 3180 |
| GGACCTGAGG CCTCTGGAGG CTACTGATGA TGCCTGCTGT GAACGCAGAC ACTGGTGTGA | 3240 |
| TGCGATGCCT GCGCCTGCAG CGGCAGTGCC CTGGGCACTA TGGTTTTGAG CTTGTACCCA | 3300 |
| GCGCTGCTTT TGCCTTGCTC TGTGACCCCA GGCAAGCTGC CTCACCTCTC TGGGCCAGTT | 3360 |
| TCCCCATTGT ACAGTGGTGC TGCACACCCT GGCCCTGGCC CCGAGGTGGC TGGGAGGTGG | 3420 |
| CTCCTCAAAC AGCCGCTGTC TCATCAGTGC CCGGTGCTGG GTCAGGGATC GACTGAGGCT | 3480 |
| CTGAGCTAAC TGGGAAACAC AGTGGCCTTG GAGGGCTGGG GAGTGTCATG GGGGTGGGGA | 3540 |
| CAGGGAGTCA CCGGTCGCAT GTGACTGAAC TCTTCACCCC AGTCTGTGGC TTTCCCGTTG | 3600 |
| CAGTGAGAGC CACGAGCCAA GGTGGGCACT TGATGTCGGA TCTCTTCAAC AAGCTGGTCA | 3660 |
| TGAGGCGCAA GGGTAGGAGG CAGGGCCGCT GCCCGCCCTG GGCCAGCACC TTGTAATTCT | 3720 |
| GTCCTGCCTT TTTCTTCCTG TATTTAAGTC TCCGGGGGCT GGGGGAACCA GGGTTTCCCA | 3780 |
| CCAACCACCC TCACTCAGCC TTTTCCCTCC AGGCATCTCT GGGAAAGGAC CTGGGGCTGG | 3840 |
| TGAGGGGCCC GGAGGAGCCT TTGCCCGCGT GTCAGACTCC ATCCCTCCTC TGCCGCCACC | 3900 |
| GCAGCAGCCA CAGGCAGAGG AGGACGAGGA CGACTGGGAA TCGTAGGGGG CTCCATGACA | 3960 |
| CCTTCCCCCC CAGACCCAGA CTTGGGCCGT TGCTCTGACA TGGACACAGC CAGGACAAGC | 4020 |
| TGCTCAGACC TACTTCCTTG GGAGGGGGTG ACGGAACCAG CACTGTGTGG AGACCAGCTT | 4080 |
| CAAGGAGCGG AAGGCTGGCT TGAGGCCACA CAGCTGGGGC GGGGACTTCT GTCTGCCTGT | 4140 |
| GCTCCATGGG GGGACGGCTC CACCCAGCCT GCGCCACTGT GTTCTTAAGA GGCTTCCAGA | 4200 |
| GAAAACGGCA CACCAATCAA TAAAGAACTG AGCAG | 4235 |
| Seq ID NO: 7 | |
| Primekey #: 421999 | |
| Coding sequence: 27..734 | |
| 1 11 21 31 41 51 | |
| \| \| \| \| \| \| | |
| GTGCAAGCAT CTGAAGAGCT GCCGGGATGC AGCAGAGAGG AGCAGCTGGA AGCCGTGGCT | 60 |
| GCGCTCTCTT CCCTCTGCTG GGCGTCCTGT TCTTCCAGGG TGTTTATATC GTCTTTTCCT | 120 |
| TGGAGATTCG TGCAGATGCC CATGTCCGAG GTTATGTTGG AGAAAAGATC AAGTTGAAAT | 180 |
| GCACTTTCAA GTCAACTTCA GATGTCACTG ACAAACTTAC TATAGACTGG ACATATCGCC | 240 |
| CTCCCAGCAG CAGCCACACA GTATCAATAT TTCATTATCA GTCTTTCCAG TACCCAACCA | 300 |
| CAGCAGGCAC ATTTCGGGAT CGGATTTCCT GGGTTGGAAA TGTATACAAA GGGGATGCAT | 360 |
| CTATAAGTAT AAGCAACCCT ACCATAAAGG ACAATGGGAC ATTCAGCTGT GCTGTGAAGA | 420 |
| ATCCCCCAGA TGTGCATCAT AATATTCCCA TGACAGAGCT AACAGTCACA GAAAGGGGTT | 480 |
| TTGGCACCAT GCTTTCCTCT GTGGCCCTTC TTTCCATCCT TGTCTTTGTG CCCTCAGCCG | 540 |
| TGGTGGTTGC TCTGCTGCTG GTGAGAATGG GGAGGAAGGC TGCTGGGCTG AAGAAGAGGA | 600 |
| GCAGGTCTGG CTATAAGAAG TCATCTATTG AGGTTTCCGA TGACACTGAT CAGGAGGAGG | 660 |
| AAGAGGCGTG TATGGCGAGG CTTTGTGTCC GTTGCGCTGA GTGCCTGGAT TCAGACTATG | 720 |
| AAGAGACATA TTGATGAAAG TCTGTATGAC ACAAGAAGAG TCACCTAAAG ACAGGAAACA | 780 |
| TCCCATTCCA CTGGCAGCTA AAGCCTGTCA GAGAAAGTGG AGCTGGCCTG GACCATAGCG | 840 |
| ATGGACAATC CTGGAGATCA TCAGTAAAGA CTTTAGGAAC CACTTATTTA TTGAATAAAT | 900 |
| GTTCTTGTTG TATTTATAAA CTGTTCAGGA ACTCTCATAA GAGACTCATG ACTTCCCCTT | 960 |
| TCAATGAATT ATGCTGTAAT TGAATGAAGA AATTCTTTTC CTGAGCAAAA AGATACTTTT | 1020 |
| TGATTCATCT TTGCTCTGGA ATGTATTACA TGTTTTCTTC CAACTGTTTG AAGGAGAATT | 1080 |
| TTGAATGTTT GCCACACCGC TGATACCCAA ATAATTTTTT AAATGAAGTG GAGCTTGTGG | 1140 |
| CTTCCTGATG TGTCACCAGA CAAAATATTC GCTTGGGATA TGTATTCTTT GTTTTTTGCT | 1200 |
| CCATGTACAC TTTCAGCTGT GAGTTAGTAT AGGGCGTATA CTTACCGGTT TAATGACCTC | 1260 |
| AACCTCAGTT GTGTTTGGAT AACTTAGGGT GTATACCCTT AGTTTCCTTA GAGTTGGTAG | 1320 |
| GATCAAGTCA TTGGTTTGCT TTGACTGGGT TTTTAAAGTA TTAAGTACAG TGTCATCAAT | 1380 |
| TTACAGTTAA GGAAAGGAAT CGTGAAGTAG AAAAATTATT TTCTTTAGTC TTGCTGGTAC | 1440 |
| AATTTGGGCT AAGGAGTCTT TGTTATTTTC TGTCTTGCTT TTTTTTTTTT TTTTTTTTTT | 1500 |
| TTGAGGCAGA GTCTCACTCT GTCGCCAGGC TGGAGTGCAG TGGTGTGATC TTGGCTCACT | 1560 |
| GCAACCTCTG CCTCCTGGGT TCAAGCGATT CTTGTGCCTC AGCCTCTCGA GTAGCTGGGA | 1620 |
| TTACAGGCAT GCGCCACCAC ACCCAGCTAA TTTTTGTGTT TTTAGTAGAG ACGGGGTTTC | 1680 |
| ACCATTTTGG CCAGGATGGT CTCAATCCCC TGACCTCGTG ATCCACCTGC CTCGGCCTCC | 1740 |
| CAAAGTGTTG GGATTACAGG CATGAGCCAC TGTGCTTGGC CTGTTATTTT ATTTTCTTAT | 1800 |
| AACTACAACT TTTCTTCTTG AATTTTCAGG TCAGAGGCAA GAAAAACTCT TTACAGGTTT | 1860 |
| TTAGTGGGGG GCTTATGGAG TATTTCAGGA GTTCTTTGCA AATTAAATCA TCTTTTCACT | 1920 |
| TGTATTGTTT TTCAAAACTT TGTTGATTTC TAAAATGTGC CAACTGTGAG TAAACTATGG | 1980 |
| TATTTGCAAG TGGTTTTTAC ATAATATTTG AGATGAGGAA GTGAGATTGT GCATGACATA | 2040 |
| CTTCTCCTTT GTATTCTCTC AGTGCCTTAC AGCAGGTTAC TCCATTCTGC TATGACAACT | 2100 |
| TGTTTCAAAT GTTAATTTAC ATAGGATTTT TTATAAGCCA TTAAGGCATA TGTATAGTAT | 2160 |
| ATCAGTAAAG ATGGATGGTG CATATATAAA TAGTCTTCTG TAATAGTGAT TGGATTTACT | 2220 |
| TCTCAATTAT GAGAGACAAA AATTATCCCC TCACCTGTCT CTATTCTTTC AACAGGTTGA | 2280 |
| TCCCTTTTCA TGATTTTTCA TTAGGTGGTT CAGGAAGTTT CCATATTACA GCGCTTCAGA | 2340 |
| CTGTATATGT TAGTTTAAAA ATCACTTTTC TCTCTCTCAA CTTCTTTCTT TTTTTTTTGA | 2400 |
| AGACTTAATT TAAAAAATTT GGGTTGTTAG ATCCGTATCA TAGATTTGGC CTAGCCTCTT | 2460 |
| CTGTTAACCT AGTCCACAGA TGAGCGAATC TGGTTAGTTG AAGGACATTG TGATTTGACT | 2520 |
| CTGGTCACGC GAGGAAGTAG AAGGGCAAAG ACAGGACCGG CAGTTTACAT TTCCAGTGGT | 2580 |
| TAAACCTCAC GGTACTTTGG GACTGCTTGT TAACTTTTGT GGTTGTCTGA GGCCAATCTA | 2640 |
| ACGTGACCAT TTCTGACACC TCAACAGAGA GAGGAAAGCA ACTTGAGCAA TGAGAGTAAA | 2700 |
| TAACTTGGGC TCTCAGAGAT TTGAAGATAG AGATCTCATT GTGAGGGGGA CTATTTTGCA | 2760 |
| GGTCCTCATT TCTCCAAGAA AGAGATGGTG TTACAGGAAC CCACTGAAAG CCATATCCCA | 2820 |
| TTAAATGAGG AACTAATTTT GGCTGGGCCT TCTTGTAATG TCCTCGCAGG TGTGTTGTGA | 2880 |
| AGATTAATGC AGGGTAGTAT GTTTGTAGAT TGACACCTAG TCTAAACTTG AGGTAATTGG | 2940 |
| TGCTCTGTGA ATACTCAGTC GTGTTCTTTT ATAGCCTTAA TCATGATTTG AACTAGTCCC | 3000 |
| TTGCTTTTTA AATGACTGAA TGAAGTCCTT CGTGGTAAGG GAGTACGTTG ATAACTTAGT | 3060 |
| TTACTATATG GGTTTGTGGT CGCATCCCAG TCATCAGCTG CTATCATTTT CCTTCTTCAT | 3120 |
| CCCTTATACT GAGATTTGGG TTACAGCTTT TTATTCTTCG AAGGATCACA AAGCAGTGTA | 3180 |
| CAGACACCTG CCTTCTTTAA GGATGAAAGG AAGATAAAGT GGTCTTTTTT TGTTTACTTA | 3240 |
| TTTGTTTCAC CTCTTGTTTG AGTAACTTCT AAGGTGCTAT TCTCTCTCTC TTTTTGCTAC | 3300 |
| CTCATGAGCT CTTGTCACAG CCATGGAAAC CAGCCTCGTT TAGAAAGGGA ACTTAGTTCA | 3360 |
| GAAGGGGTTA AAAGCCTTCC AGAATTTTTC TTTAGCTGCT GAAGTTTTTA CATGTGGTTA | 3420 |
| CATGACTTTA AGTTTTATGC ATTACGCTCT TAATTCTATT ACAAAATGTG GACTCACCAA | 3480 |
| TTGCTTTGTG TTTTCCATGT GACCTGTTAC TTCAGGCTAC TTGGGGAACA TCTTAGTCCT | 3540 |
| CTGTAGCTCC TGAACCCAGC ACTGGTGCTT CAAGAGAGAA GGTAGCACGT CTTTGTTCAA | 3600 |
| AACAAAACAA AACGACACTT CTGGAGGCCA CATCCTGAAT ATGAATGTTC TACTAAGTCA | 3660 |
| CTCAGTTATG GTTCTAAAGG GAAACTGTAA GAAGACCCAC AAGGAGTGGA CCAAGACTAT | 3720 |
| TATTTAATTG CACAACTTGA AACTTTGCTG CCAGAAGAGG CAGCTCCATT CCTTTGACTC | 3780 |
| CAGTGTTGGG CTGTTAACTG CTGCACCTCA TTGCCTTTTT TTGTTTTTGT TTTTGTTTTG | 3840 |
| TAGGAGGGTA GGCACTGTTG GGCCATATGC ACAAATATTG TAACTCTTGG TATCTTTACT | 3900 |
| GCATCATAGT CAATAAACTT CTTTGTACCC TT | 3932 |
| Seq ID NO: 8 | |
| Primekey #: 445909 | |
| Coding sequence: 83..898 | |
| 1 11 21 31 41 51 | |
| \| \| \| \| \| \| | |
| GGCACGAGGC GGGCCAGCGA CGGGCAGGAC GCCCCGTTCG CCTAGCGCGT GCTCAGGAGT | 60 |
| TGGTGTCCTG CCTGCGCTCA GGATGAGGGG GAATCTGGCC CTGGTGGGCG TTCTAATCAG | 120 |
| CCTGGCCTTC CTGTCACTGC TGCCATCTGG ACATCCTCAG CCGGCTGGCG ATGACGCCTG | 180 |
| CTCTGTGCAG ATCCTCGTCC CTGGCCTCAA AGGGGATGCG GGAGAGAAGG GAGACAAAGG | 240 |
| CGCCCCCGGA CGGCCTGGAA GAGTCGGCCC CACGGGAGAA AAAGGAGACA TGGGGGACAA | 300 |
| AGGACAGAAA GGCAGTGTGG GTCGTCATGG AAAAATTGGT CCCATTGGCT CTAAAGGTGA | 360 |
| GAAAGGAGAT TCCGGTGACA TAGGACCCCC TGGTCCTAAT GGAGAACCAG GCCTCCCATG | 420 |
| TGAGTGCAGC CAGCTGCGCA AGGCCATCGG GGAGATGGAC AACCAGGTCT CTCAGCTGAC | 480 |
| CAGCGAGCTC AAGTTCATCA AGAATGCTGT CGCCGGTGTG CGCGAGACGG AGAGCAAGAT | 540 |
| CTACCTGCTG GTGAAGGAGG AGAAGCGCTA CGCGGACGCC CAGCTGTCCT GCCAGGGCCG | 600 |
| CGGGGGCACG CTGAGCATGC CCAAGGACGA GGCTGCCAAT GGCCTGATGG CCGCATACCT | 660 |
| GGCGCAAGCC GGCCTGGCCC GTGTCTTCAT CGGCATCAAC GACCTGGAGA AGGAGGGCGC | 720 |
| CTTCGTGTAC TCTGACCACT CCCCCATGCG GACCTTCAAC AAGTGGCGCA GCGGTGAGCC | 780 |
| CAACAATGCC TACGACGAGG AGGACTGCGT GGAGATGGTG GCCTCGGGCG GCTGGAACGA | 840 |
| CGTGGCCTGC CACACCACCA TGTACTTCAT GTGTGAGTTT GACAAGGAGA ACATGTGAGC | 900 |
| CTCAGGCTGG GGCTGCCCAT TGGGGGCCCC ACATGTCCCT GCAGGGTTGG CAGGGACAGA | 960 |
| GCCCAGACCA TGGTGCCAGC CAGGGAGCTG TCCCTCTGTG AAGGGTGGAG GCTCACTGAG | 1020 |
| TAGAGGGCTG TTGTCTAAAC TGAGAAAATG GCCTATGCTT AAGAGGAAAA TGAAAGTGTT | 1080 |
| CCTGGGGTGC TGTCTCTGAA GAAGCAGAGT TTCATTACCT GTATTGTAGC CCCAATGTCA | 1140 |
| TTATGTAATT ATTACCCAGA ATTGCTCTTC CATAAAGCTT GTGCCTTTGT CCAAGCTATA | 1200 |
| CAATAAAATC TTTAAGTAGT GCAGTAAAAA AAAAAAAAAA AAAAAAAAAA AAAAAAA | 1257 |
| Seq ID NO: 9 | |
| Primekey #: 450628 | |
| Coding sequence: 80..2305 | |
| 1 11 21 31 41 51 | |
| \| \| \| \| \| \| | |
| CAATGCTACA TTAACCCATT ATGTAAGACC AATAAATGCA GAGCCAGCGT TTCAAGCACA | 60 |
| GGAAATACCA GCAGGCAGAA TGGCCAGTTT GCTTAAGAAT GGTGAGCCTG AAGCTGAGTT | 120 |
| ACATAAAGAA ACCACAGGTC CAGGCACTGC TGGCCCTCAG TCCAACACCA CATCTTCTCT | 180 |
| AAAAGGTGAA CGCAAAGCCA TCCACACGCT GCAAGATGTG TCAACATGTG AAACAAAGGA | 240 |
| GCTATTGAAT GTCGGGGTTT CCTCCCTTTG TGCTGGTCCC TACCAAAATA CAGCAGACAC | 300 |
| CAAGGAAAAC CTCAGTAAAG AGCCTTTGGC CTCCTTTGTT TCAGAATCCT TTGATACTTC | 360 |
| TGTTTGTGGA ATAGCCACAG AGCACGTAGA AATTGAGAAC AGTGGGGAGG GGCTCAGGGC | 420 |
| TGAGGCTGGT TCTGAAACCC TAGGCAGAGA TGGAGAGGTC GGTGTGAATT CCGACATGCA | 480 |
| CTATGAACTC TCTGGAGATT CTGATCTAGA CCTGCTTGGT GATTGTAGAA ATCCCAGACT | 540 |
| GGATTTGGAG GATTCTTATA CTTTAAGAGG TAGTTACACC AGGAAAAAAG ATGTTCCCAC | 600 |
| AGATGGCTAT GAGTCGTCGT TGAACTTCCA CAACAACAAC CAAGAGGACT GGGGCTGCTC | 660 |
| TAGCCGGGTT CCAGGCATGG AGACGAGCCT CCCTCCCGGG CACTGGACTG CTGCGGTAAA | 720 |
| GAAAGAAGAG AAGTGTGTGC CGCCTTACGT CCAAATCCGA GATCTCCACG GGATCCTCAG | 780 |
| GACTTACGCC AACTTCTCTA TAACAAAAGA ACTCAAAGAT ACCATGAGAA CTTCACACGG | 840 |
| CCTGAGGAGG CACCCGAGTT TCAGTGCAAA CTGTGGCCTG CCCAGCTCCT GGACAAGCAC | 900 |
| TTGGCAGGTG GCAGACGACC TCACCCAGAA CACTTTAGAC CTGGAGTATC TGCGTTTTGC | 960 |
| ACATAAACTA AAACAGACCA TAAAGAATGG GGATTCTCAG CATTCTGCCT CCTCTGCCAA | 1020 |
| TGTCTTTCCA AAGGAGTCAC CAACCCAGAT CTCCATTGGT GCTTTCCCTT CGACAAAAAT | 1080 |
| CTCTGAGGCC CCATTTCTGC ATCCTGCACC TAGGAGCAGA AGCCCCCTTC TGGTAACAGC | 1140 |
| TGTGGAGTCA GATCCCAGAC CACAGGGACA GCCCAGGAGA GGCTACACAG CCAGCAGTCT | 1200 |
| GGACATCTCT TCCTCTTGGA GAGAGAGATG TAGTCATAAT AGAGATCTTA GAAATTCTCA | 1260 |
| AAGAAATCAC ACTGTTTCAT TCCACCTCAA CAAACTGAAA TACAACAGTA CTGTGAAGGA | 1320 |
| ATCTCGGAAT GATATTTCAC TTATTCTCAA TGAGTATGCT GAATTCAACA AGGTGATGAA | 1380 |
| GAATAGCAAC CAATTCATTT TCCAAGACAA AGAGCTAAAT GATGTTTCTG GAGAAGCCAC | 1440 |
| TGCTCAAGAG ATGTATCTGC CTTTCCCAGG ACGGTCAGCC TCCTATGAAG ACATAATCAT | 1500 |
| AGACGTGTGC ACCAATTTGC ACGTCAAACT AAGAAGTGTT GTGAAAGAGG CTTGTAAAAG | 1560 |
| TACCTTCCTG TTCTACCTTG TCGAAACAGA AGACAAATCA TTCTTTGTAA GAACAAAGAA | 1620 |
| CCTTCTGAGG AAAGGAGGCC ATACAGAAAT TGAACCTCAG CACTTCTGTC AAGCTTTCCA | 1680 |
| CAGAGAGAAT GATACACTAA TCATCATCAT CAGAAATGAA GATATATCAT CACATTTGCA | 1740 |
| TCAGATTCCT TCTTTGCTGA AGCTGAAGCA TTTCCCCAGT GTCATCTTTG CTGGAGTAGA | 1800 |
| CAGCCCTGGA GATGTTCTTG ATCACACCTA CCAAGAACTG TTTCGTGCAG GAGGCTTTGT | 1860 |
| GATATCAGAT GACAAGATAC TAGAAGCTGT AACATTAGTT CAACTGAAGG AAATTATCAA | 1920 |
| AATCCTGGAA AAACTAAATG GAAATGGAAG ATGGAAGTGG TTGCTTCACT ACAGGGAAAA | 1980 |
| TAAAAAGCTA AAAGAAGATG AAAGAGTGGA TTCAACTGCA CATAAGAAGA ACATAATGTT | 2040 |
| GAAGTCATTT CAGAGTGCAA ATATCATTGA ATTGCTTCAT TATCACCAGT GTGACTCTCG | 2100 |
| ATCATCAACA AAAGCAGAAA TTCTGAAATG TTTGCTAAAC CTGCAAATTC AGCATATTGA | 2160 |
| TGCCAGGTTT GCTGTCCTCC TAACAGACAA GCCTACTATC CCCAGAGAAG TCTTTGAAAA | 2220 |
| TAGTGGAATC CTTGTTACAG ATGTAAATAA CTTTATAGAA AACATAGAAA AAATAGCAGC | 2280 |
| TCCATTTAGG AGTAGCTATT GGTGACTCAA CTACAGCCTG CCTGGATATG GATGATGCCA | 2340 |
| ATAAAAAATT AGTATTTTCC CTTTGGAAAA CTTGTGAACA TGTGAATACA CATGTGAAGT | 2400 |
| CTTACATTTG AAAAACCAAT GTTCTACAAC TTGGAAAGTT TTCATTTTTT ATATTTTGCT | 2460 |
| GAAATATGTC ACAGTGGCAT TGCAGTTGTC TGTTAGCTTT GGGTTGCAGT GCTAGATATT | 2520 |
| GTTTTAAATT ATTTTCATTT TAAACAAGAT GCCTTCTAAG CTATTGAGCT TATTAAAAAT | 2580 |
| AATTTTACAT GTTTACTTAG TTGGAGCAAA AATAAGTCTA TTTTAACGAA TAGCTTTGTT | 2640 |
| TTTGCTATGC TAATGTCTAG AAAGGCATAC GATGCTACTA TTATGCTCTG TTTTAAAGGT | 2700 |
| TTTACCTACC CTTGTAAAAA CTATAATCTT AAATGGTTTT ATTTGCTGTT TACTACTTAT | 2760 |
| ACATACTACT ACTATAAAAC TATTTTTTCC TAAATGGTAC AAATTTATAA ACTATCATTT | 2820 |
| TTCACTTACG GTATTTGTAA ATACTACTAC TACAAAAATC AGCTTTCCGA GAAAGAAATA | 2880 |
| ATCATTTATT TATGATATTG AAAATTTCTA CAGTAAACAC TCAAAACCAA GCAAAAAACA | 2940 |
| TTTGTAAGAT ACACGGTATC TATTTGGAGC AACGGTTTTT GTAACTAATG TGTTTCATTT | 3000 |
| TTTAAATAAA GACAACTAAA AATAAAAAAA AAAAAAAAAA A | 3041 |
| Seq ID NO: 10 | |
| Primekey #: 408806 | |
| Coding sequence: 80..3430 | |
| 1 11 21 31 41 51 | |
| \| \| \| \| \| \| | |
| TGCCCAGGAG GAGTAGGAGC AGGAGCAGAA GCAGAAGCGG GGTCCGGAGC TGCGCGCCTA | 60 |
| CGCGGGACCT GTGTCCGAAA TGCCGGTGCG AGGAGACCGC GGGTTTCCAC CCCGGCGGGA | 120 |
| GCTGTCAGGT TGGCTCCGCG CCCCAGGCAT GGAAGAGCTG ATATGGGAAC AGTACACTGT | 180 |
| GACCCTACAA AAGGATTCCA AAAGAGGATT TGGAATTGCA GTGTCCGGAG GCAGAGACAA | 240 |
| CCCCCACTTT GAAAATGGAG AAACGTCAAT TGTCATTTCT GATGTGCTCC CGGGTGGGCC | 300 |
| TGCTGATGGG CTGCTCCAAG AAAATGACAG AGTGGTCATG GTCAATGGCA CCCCCATGGA | 360 |
| GGATGTGCTT CATTCGTTTG CAGTTCAGCA GCTCAGAAAA AGTGGGAAGG TCGCTGCTAT | 420 |
| TGTGGTCAAG AGGCCCCGGA AGGTCCAGGT GGCCGCACTT CAGGCCAGCC CTCCCCTGGA | 480 |
| TCAGGATGAC CGGGCTTTTG AGGTGATGGA CGAGTTTGAT GGCAGAAGTT TCCGGAGTGG | 540 |
| CTACAGCGAG AGGAGCCGGC TGAACAGCCA TGGGGGGCGC AGCCGCAGCT GGGAGGACAG | 600 |
| CCCGGAAAGG GGGCGTCCCC ATGAGCGGGC CCGGAGCCGG GAGCGGGACC TCAGCCGGGA | 660 |
| CCGGAGCCGT GGCCGGAGCC TGGAGCGGGG CCTGGACCAA GACCATGCGC GCACCCGAGA | 720 |
| CCGCAGCCGT GGCCGGAGCC TGGAGCGGGG CCTGGACCAC GACTTTGGGC CATCCCGGGA | 780 |
| CCGGGACCGT GACCGCAGCC GCGGCCGGAG CATTGACCAG GACTACGAGC GAGCCTATCA | 840 |
| CCGGGCCTAC GACCCAGACT ACGAGCGGGC CTACAGCCCG GAGTACAGGC GCGGGGCCCG | 900 |
| CCACGATGCC CGCTCTCGGG GACCCCGAAG CCGCAGCCGC GAGCACCCGC ACTCACGGAG | 960 |
| CCCCAGCCCC GAGCCTAGGG GGCGGCCGGG GCCCATCGGG GTCCTCCTGA TGAAAAGCAG | 1020 |
| AGCGAACGAA GAGTATGGTC TCCGGCTTGG GAGTCAGATC TTCGTAAAGG AAATGACCCG | 1080 |
| AACGGGTCTG GCAACTAAAG ATGGCAACCT TCACGAAGGA GACATAATTC TCAAGATCAA | 1140 |
| TGGGACTGTA ACTGAGAACA TGTCTTTAAC GGATGCTCGA AAATTGATAG AAAAGTCAAG | 1200 |
| AGGAAAACTA CAGCTAGTGG TGTTGAGAGA CAGCCAGCAG ACCCTCATCA ACATCCCGTC | 1260 |
| ATTAAATGAC AGTGACTCAG AAATAGAAGA TATTTCAGAA ATAGAGTCAA CCCGATCATT | 1320 |
| TTCTCCAGAG GAGAGACGTC ATCAGTATTC TGATTATGAT TATCATTCCT CAAGTGAGAA | 1380 |
| GCTGAAGGAA AGGCCAAGTT CCAGAGAGGA CACGCCGAGC AGATTGTCCA GGATGGGTGC | 1440 |
| GACACCCACT CCCTTTAAGT CCACAGGGGA TATTGCAGGC ACAGTTGTCC CAGAGACCAA | 1500 |
| CAAGGAACCC AGATACCAAG AGGAACCCCC AGCTCCTCAA CCAAAAGCAG CCCCGAGAAC | 1560 |
| TTTTCTTCGT CCTAGTCCTG AAGATGAAGC AATATATGGC CCTAATACCA AAATGGTAAG | 1620 |
| GTTCAAGAAG GGAGACAGCG TGGGCCTCCG GTTGGCTGGT GGCAATGATG TCGGGATATT | 1680 |
| TGTTGCTGGC ATTCAAGAAG GGACCTCGGC GGAGCAGGAG GGCCTTCAAG AAGGAGACCA | 1740 |
| GATTCTGAAG GTGAACACAC AGGATTTCAG AGGATTAGTG CGGGAGGATG CCGTTCTCTA | 1800 |
| CCTGTTAGAA ATCCCTAAAG GTGAAATGGT GACCATTTTA GCTCAGAGCC GAGCCGATGT | 1860 |
| GTATAGAGAC ATCCTGGCTT GTGGCAGAGG GGATTCGTTT TTTATAAGAA GCCACTTTGA | 1920 |
| ATGTGAGAAG GAAACTCCAC AGAGCCTGGC CTTCACCAGA GGGGAGGTCT TCCGAGTGGT | 1980 |
| AGACACACTG TATGACGGCA AGCTGGGCAA CTGGCTGGCT GTGAGGATTG GGAACGAGTT | 2040 |
| GGAGAAAGGC TTAATCCCCA ACAAGAGCAG AGCTGAACAA ATGGCCAGTG TTCAAAATGC | 2100 |
| CCAGAGAGAC AACGCTGGGG ACCGGGCAGA TTTCTGGAGA ATGCGTGGCC AGAGGTCTGG | 2160 |
| GGTGAAGAAG AACCTGAGGA AAAGTCGGGA AGACCTCACA GCTGTTGTGT CTGTCAGCAC | 2220 |
| CAAGTTCCCA GCTTATGAGA GGGTTTTGCT GCGAGAAGCT GGTTTCAAGA GACCTGTGGT | 2280 |
| CTTATTCGGC CCCATAGCTG ATATAGCAAT GGAAAAATTG GCTAATGAGT TACCTGACTG | 2340 |
| GTTTCAAACT GCTAAAACGG AACCAAAAGA TGCAGGATCT GAGAAATCCA CTGGAGTGGT | 2400 |
| CCGGTTAAAT ACCGTGAGGC AAGTTATTGA ACAGGATAAG CATGCACTAC TGGATGTGAC | 2460 |
| TCCGAAAGCT GTGGACCTGT TGAATTACAC CCAGTGGTTC TCAATTGTGA TTTCTTTCAC | 2520 |
| GCCAGACTCC AGACAAGGTG TCAACACCAT GAGACAAAGG TTAGACCCAA CGTCCAACAA | 2580 |
| TAGTTCTCGA AAGTTATTTG ATCACGCCAA CAAGCTTAAA AAAACGTGTG CACACCTTTT | 2640 |
| TACAGCTACA ATCAACCTAA ATTCAGCCAA TGATAGCTGG TTTGGCAGCT TAAAGGACAC | 2700 |
| TATTCAGCAT CAGCAAGGAG AAGCGGTTTG GGTCTCTGAA GGAAAGATGG AAGGGATGGA | 2760 |
| TGATGACCCC GAAGACCGCA TGTCCTACTT AACTGCCATG GGCGCAGACT ATCTGAGTTG | 2820 |
| CGACAGCCGC CTCATCAGTG ACTTTGAAGA CACGGACGGT GAAGGAGGCG CCTACACTGA | 2880 |
| CAATGAGCTG GATGAGCCAG CCGAGGAGCC GCTGGTGTCG TCCATCACCC GCTCCTCGGA | 2940 |
| GCCGGTGCAG CACGAGGAGA GCATAAGGAA ACCCAGCCCA GAGCCACGAG CTCAGATGAG | 3000 |
| GAGGGCTGCT AGCAGCGATC AACTTAGGGA CAATAGCCCG CCCCCAGCAT TCAAGCCAGA | 3060 |
| GCCGTCCAAG GCCAAAACCC AGAACAAAGA AGAATCCTAT GACTTCTCCA AATCCTATGA | 3120 |
| ATATAAGTCA AACCCCTCTG CCGTTGCTGG TAATGAAACT CCTGGGGCAT CTACCAAAGG | 3180 |
| TTATCCTCCT CCTGTTGCAG CAAAACCTAC CTTTGGGCGG TCTATACTGA AGCCCTCCAC | 3240 |
| TCCCATCCCT CCTCAAGAGG GTGAGGAGGT GGGAGAGAGC AGTGAGGAGC AAGATAATGC | 3300 |
| TCCCAAATCA GTCCTGGGCA AAGTCAAAAT ATTTGGAGAA GATGGATCAC AAGGGCCAGG | 3360 |
| GTTACAAGAG AATGCAGGAG CTCCAGGAAG CACAGAATGC AAGGATCGAA ATTGCCCAGA | 3420 |
| AGCATCCTGA TATCTATGCA GTTCCAATCA AAACGCACAA GCCAGACCCT GGCACGCCCC | 3480 |
| AGCACACGAG TTCCAGACCC CCTGAGCCAC AGAAAGCTCC TTCCAGACCT TATCAGGATA | 3540 |
| CCAGAGGAAG TTATGGCAGT GATGCCGAGG AGGAGGAGTA CCGCCAGCAG CTGTCAGAAC | 3600 |
| ACTCCAAGCG CGGTTACTAT GGCCAGTCTG CCCGATACCG GGACACAGAA TTATAGATGT | 3660 |
| CTGAGCACGG ACTCTCCCAG GCCTGCCTGC ATGGCATCAG ACTAGCCACT CCTGCCAGGC | 3720 |
| CGCCGGGATG GTTCTTCTCC AGTTAGAATG CACCATGGAG ACGTGGTGGG ACTCCAGCTC | 3780 |
| GTGTGTCCTC ATGGAGAACC CAGGGGACAG CTGGTGCAAA TTCAGAACTG AGGGCTCTGT | 3840 |
| TTGTGGGACT GGGTTAGAGG AGTCTGTGGC TTTTTGTTCA GAATTAAGCA GAACACTGCA | 3900 |
| GTCAGATCCT GTTACTTGCT TCAGTGGACC GAAATCTGTA TTCTGTTTGC GTACTTGTAA | 3960 |
| TATGTATATT AAGAAGCAAT AACTATTTTT CCTCATTAAT AGCTGCCTTC AAGGACTGTT | 4020 |
| TCAGTGTGAG TCAGAATGTG AAAAAGGAAT AAAAAATACT GTTGGGCTCA AACTAAATTC | 4080 |
| AAAGAAGTAC TTTATTGCAA CTCTTTTAAG TGCCTTGGAT GAGAAGTGTC TTAAATTTTC | 4140 |
| TTCCTTTGAA GCTTTAGGCA GAGCCATAAT GGACTAAAAC ATTTTGACTA AGTTTTTATA | 4200 |
| CCAGCTTAAT AGCTGTAGTT TTCCCTGCAC TGTGTCATCT TTTCAAGGCA TTTGTCTTTG | 4260 |
| TAATATTTTC CATAAATTTG GACTGTCTAT ATCATAACTA TACTTGATAG TTTGGCTATA | 4320 |
| AGTGCTCAAT AGCTTGAAGC CCAAGAAGTT GGTATCGAAA TTTGTTGTTT GTTTAAACCC | 4380 |
| AAGTGCTGCA CAAAAGCAGA TACTTGAGGA AAACACTATT TCCAAAAGCA CATGTATTGA | 4440 |
| CAACAGTTTT ATAATTTAAT AAAAAGGAAT ACATTGCAAT CCGT | 4484 |
| Seq ID NO: 11 | |
| Primekey #: 408806 | |
| Coding sequence: 80..3061 | |
| 1 11 21 31 41 51 | |
| \| \| \| \| \| \| | |
| TGCCCAGGAG GAGTAGGAGC AGGAGCAGAA GCAGAAGCGG GGTCCGGAGC TGCGCGCCTA | 60 |
| CGCGGGACCT GTGTCCGAAA TGCCGGTGCG AGGAGACCGC GGGTTTCCAC CCCGGCGGGA | 120 |
| GCTGTCAGGT TGGCTCCGCG CCCCAGGCAT GGAAGAGCTG ATATGGGAAC AGTACACTGT | 180 |
| GACCCTACAA AAGGATTCCA AAAGAGGATT TGGAATTGCA GTGTCCGGAG GCAGAGACAA | 240 |
| CCCCCACTTT GAAAATGGAG AAACGTCAAT TGTCATTTCT GATGTGCTCC CGGGTGGGCC | 300 |
| TGCTGATGGG CTGCTCCAAG AAAATGACAG AGTGGTCATG GTCAATGGCA CCCCCATGGA | 360 |
| GGATGTGCTT CATTCGTTTG CAGTTCAGCA GCTCAGAAAA AGTGGGAAGG TCGCTGCTAT | 420 |
| TGTGGTCAAG AGGCCCCGGA AGGTCCAGGT GGCCGCACTT CAGGCCAGCC CTCCCCTGGA | 480 |
| TCAGGATGAC CGGGCTTTTG AGGTGATGGA CGAGTTTGAT GGCAGAAGTT TCCGGAGTGG | 540 |
| CTACAGCGAG AGGAGCCGGC TGAACAGCCA TGGGGGGCGC AGCCGCAGCT GGGAGGACAG | 600 |
| CCCGGAAAGG GGGCGTCCCC ATGAGCGGGC CCGGAGCCGG GAGCGGGACC TCAGCCGGGA | 660 |
| CCGGAGCCGT GGCCGGAGCC TGGAGCGGGG CCTGGACCAA GACCATGCGC GCACCCGAGA | 720 |
| CCGCAGCCGT GGCCGGAGCC TGGAGCGGGG CCTGGACCAC GACTTTGGGC CATCCCGGGA | 780 |
| CCGGGACCGT GACCGCAGCC GCGGCCGGAG CATTGACCAG GACTACGAGC GAGCCTATCA | 840 |
| CCGGGCCTAC GACCCAGACT ACGAGCGGGC CTACAGCCCG GAGTACAGGC GCGGGGCCCG | 900 |
| CCACGATGCC CGCTCTCGGG GACCCCGAAG CCGCAGCCGC GAGCACCCGC ACTCACGGAG | 960 |
| CCCCAGCCCC GAGCCTAGGG GGCGGCCGGG GCCCATCGGG GTCCTCCTGA TGAAAAGCAG | 1020 |
| AGCGAACGAA GAGTATGGTC TCCGGCTTGG GAGTCAGATC TTCGTAAAGG AAATGACCCG | 1080 |
| AACGGGTCTG GCAACTAAAG ATGGCAACCT TCACGAAGGA GACATAATTC TCAAGATCAA | 1140 |
| TGGGACTGTA ACTGAGAACA TGTCTTTAAC GGATGCTCGA AAATTGATAG AAAAGTCAAG | 1200 |
| AGGAAAACTA CAGCTAGTGG TGTTGAGAGA CAGCCAGCAG ACCCTCATCA ACATCCCGTC | 1260 |
| ATTAAATGAC AGTGACTCAG AAATAGAAGA TATTTCAGAA ATAGAGTCAA CCCGATCATT | 1320 |
| TTCTCCAGAG GAGAGACGTC ATCAGTATTC TGATTATGAT TATCATTCCT CAAGTGAGAA | 1380 |
| GCTGAAGGAA AGGCCAAGTT CCAGAGAGGA CACGCCGAGC AGATTGTCCA GGATGGGTGC | 1440 |
| GACACCCACT CCCTTTAAGT CCACAGGGGA TATTGCAGGC ACAGTTGTCC CAGAGACCAA | 1500 |
| CAAGGAACCC AGATACCAAG AGGAACCCCC AGCTCCTCAA CCAAAAGCAG CCCCGAGAAC | 1560 |
| TTTTCTTCGT CCTAGTCCTG AAGATGAAGC AATATATGGC CCTAATACCA AAATGGTAAG | 1620 |
| GTTCAAGAAG GGAGACAGCG TGGGCCTCCG GTTGGCTGGT GGCAATGATG TCGGGATATT | 1680 |
| TGTTGCTGGC ATTCAAGAAG GGACCTCGGC GGAGCAGGAG GGCCTTCAAG AAGGAGACCA | 1740 |
| GATTCTGAAG GTGAACACAC AGGATTTCAG AGGATTAGTG CGGGAGGATG CCGTTCTCTA | 1800 |
| CCTGTTAGAA ATCCCTAAAG GTGAAATGGT GACCATTTTA GCTCAGAGCC GAGCCGATGT | 1860 |
| GTATAGAGAC ATCCTGGCTT GTGGCAGAGG GGATTCGTTT TTTATAAGAA GCCACTTTGA | 1920 |
| ATGTGAGAAG GAAACTCCAC AGAGCCTGGC CTTCACCAGA GGGGAGGTCT TCCGAGTGGT | 1980 |
| AGACACACTG TATGACGGCA AGCTGGGCAA CTGGCTGGCT GTGAGGATTG GGAACGAGTT | 2040 |
| GGAGAAAGGC TTAATCCCCA ACAAGAGCAG AGCTGAACAA ATGGCCAGTG TTCAAAATGC | 2100 |
| CCAGAGAGAC AACGCTGGGG ACCGGGCAGA TTTCTGGAGA ATGCGTGGCC AGAGGTCTGG | 2160 |
| GGTGAAGAAG AACCTGAGGA AAAGTCGGGA AGACCTCACA GCTGTTGTGT CTGTCAGCAC | 2220 |
| CAAGTTCCCA GCTTATGAGA GGGTTTTGCT GCGAGAAGCT GGTTTCAAGA GACCTGTGGT | 2280 |
| CTTATTCGGC CCCATAGCTG ATATAGCAAT GGAAAAATTG GCTAATGAGT TACCTGACTG | 2340 |
| GTTTCAAACT GCTAAAACGG AACCAAAAGA TGCAGGATCT GAGAAATCCA CTGGAGTGGT | 2400 |
| CCGGTTAAAT ACCGTGAGGC AAGTTATTGA ACAGGATAAG CATGCACTAC TGGATGTGAC | 2460 |
| TCCGAAAGCT GTGGACCTGT TGAATTACAC CCAGTGGTTC CCAATTGTGA TTTTTTTCAA | 2520 |
| CCCAGACTCC AGACAAGGTG TCAAAACCAT GAGACAAAGG TTAAATCCAA CGTCCAACAA | 2580 |
| AAGTTCTCGA AAGTTATTTG ATCAAGCCAA CAAGCTTAAA AAAACGTGTG CACACCTTTT | 2640 |
| TACAGCTACA ATCAACCTAA ATTCAGCCAA TGATAGCTGG TTTGGCAGCT TAAAGGACAC | 2700 |
| TATTCAGCAT CAGCAAGGAG AAGCGGTTTG GGTCTCTGAA GGAAAGATGG AAGGGATGGA | 2760 |
| TGATGACCCC GAAGACCGCA TGTCCTACTT AACCGCCATG GGCGCGGACT ATCTGAGTTG | 2820 |
| CGACAGCCGC CTCATCAGTG ACTTTGAAGA CACGGACGGT GAAGGAGGCG CCTACACTGA | 2880 |
| CAATGAGCTG GATGAGCCAG CCGAGGAGCC GCTGGTGTCG TCCATCACCC GCTCCTCGGA | 2940 |
| GCCGGTGCAG CACGAGGAGG TGAGGCGAGG CAGGCCACGG GCAGGAACAG GAGAGCCTGG | 3000 |
| TGTTTTCCTT GCACTCTCGT GGACAGCTGT GTGTTCAGGG TGCTGTGGAA GGCATTCCTA | 3060 |
| AGGGTTGGAG CAGATGACTT CCAGGGAGTC TCTCGCTTTG AGTCCACGCT GGCATGGTTG | 3120 |
| CAGTCTGTGG GGAAAGTGGG GCAGGCAGGT GGACTTCAGA AGAGCTTGGA GGGGTCAGCA | 3180 |
| CTCCGCACAC CCATGCCCTC AGGTGCGATG GATAAACAGA ATGGCTTTAG GTGCCGTCTG | 3240 |
| TCCAAATTAC CAGCGGAACC TTCCTTCCCA TGCAGTATTG TTGTATGTAC TTGTAACCTT | 3300 |
| TGATTAGGTT TCTCTCTGTA CTCTTAGATG TCCTTGCTTT TCTTCCCCAT CCTGCCTTTA | 3360 |
| ACCTTTCTAA TCTTGCCAAA GCTCTTGAGT GTTTCCCCAT CAGTTTCCTT CTCTCTTATA | 3420 |
| TTTCAGTTTT TTAATTGAGT TCATGATCAA ACCTTCATCT GATCACATCA CATGTACTGT | 3480 |
| GCATCCACTG TGATTAGATA GCTTATGGGA TCCTTGAAAT CACATTGACA GGCACTGTAA | 3540 |
| AGTCACAGCC AAGTTAGCAA TTATTAGTTG CACCTCAGAG AATGTTGGAA TAATGATCTT | 3600 |
| TGAAGATGGG ATTGTTCATA TATTTGGATA ATTATTGCTG TGGATTTCTC TCTAGCATTT | 3660 |
| TAGCTCATTC CAGTAAATGA TTTTTTTCTT TATGAAATAG AACTCCCAAA AAAAAAAAAA | 3720 |
| AAAAAAAAA | 3729 |
| Seq ID NO: 12 | |
| Primekey #: 407584 | |
| Coding sequence: 95..535 | |
| 1 11 21 31 41 51 | |
| \| \| \| \| \| \| | |
| CAAGCCTGGA AGAACTCGTC ATGCTCTTTG TAGCGTGGTG CTTCTGTTGC TCACAGGACA | 60 |
| ACTTGCCTTT GATGATTTTC AAGAGAGTTG TGCTATGATG TGGCAAAAGT ATGCAGGAAG | 120 |
| CAGGCGGTCA ATGCCTCTGG GAGCAAGGAT CCTTTTCCAC GGTGTGTTCT ATGCCGGGGG | 180 |
| CTTTGCCATT GTGTATTACC TCATTCAAAA GTTTCATTCC AGGGCTTTAT ATTACAAGTT | 240 |
| GGCAGTGGAG CAGCTGCAGA GCCATCCCGA GGCACAGGAA GCTCTGGGCC CTCCTCTCAA | 300 |
| CATCCATTAT CTCAAGCTCA TCGACAGGGA AAACTTCGTG GACATTGTTG ATGCCAAGTT | 360 |
| GAAGATTCCT GTCTCTGGAT CCAAATCAGA GGGCCTTCTC TACGTCCACT CATCCAGAGG | 420 |
| TGGCCCCTTT CAGAGGTGGC ACCTTGACGA GGTCTTTTTA GAGCTCAAGG ATGGTCAGCA | 480 |
| GATTCCTGTG TTCAAGCTCA GTGGGGAAAA CGGTGATGAA GTGAAAAAGG AGTAGAGACG | 540 |
| ACCCAGAAGA CCCAGCTTGC TTCTAGTCCA TCCTTCCCTC ATCTCTACCA TATGGCCACT | 600 |
| GGGGTGGTGG CCCATCTCAG TGACAGACAC TCCTGCAACC CAGTTTTCCA GCCACCAGTG | 660 |
| GGATGATGGT ATGTGCCAGC ACATGGTAAT TTTGGTGTAA TTCTAACTTG GGCACAACAA | 720 |
| ATGCTATTTG TCATTTTTAA ACTGAATCCG AAAGAAACTC CTATTATAAA TTTAAGATAA | 780 |
| TGTAATGTAT TTGAAAGTGC TTTGTATAAA AAAGCACATG ATAAAAGGAA TCAGAATTAA | 840 |
| TAAAATGTTT GTTGATCTTT AAAAAAAAAA AAAAAAAAAC TCGAGACTAG TTCTGTCTCT | 900 |
| CCCTCGTGCC GAATTCGGCA CGAGGCAGAG CCTCTTCTCG TCTGTAGGAA CACCGCCAGG | 960 |
| GAGGTCATGG CAGGGCAGGA CCAAAGGGTC CTGTGGCTCT TTTTTTTTCT CCTGTTCTGC | 1020 |
| ATTCCTGCCC ACACCCCCAC CCCTCCATTT CCTTCTGCTC TGGAGGCATC CTCCTTCATT | 1080 |
| GGACACCACA CAGTTTATTT CACTTCTGAC TTCAAGGTTG TGAATTCTTC CCATGGCTTA | 1140 |
| AGTCCTGGGA TACTTCTGCA GTGAAAGGAG GTCTTGTACC TCTTCCTCAG AGTCAGAAGT | 1200 |
| TCTGAGTACC TTTGCCCTAT TCTGAAAAGG GCTAGGGGCT CCTGCTCCCA GCTGCCCTCT | 1260 |
| TCCTTTGGCT TCCAATTCAG TTCCCTCTGC CCCGCATCCT GCAGACAGGC GCTCCCGCAG | 1320 |
| GGGGCCCTTG TGGACCTGCA CTGGAGTCTG TTGCCTTCAC TGAGCTGCCT GTGCTGGCCT | 1380 |
| TGCATGGTGC CTGTAGGGGG ATTTGCTTTG CTGTGCCATT GGGGTACAGC TGCTGCTCTT | 1440 |
| ACTCTAGACC AAAAAGTCGG GTTGAGTGAC TGGTGGCAGG GCCACAGATA GAGACAGCGG | 1500 |
| GGAGGGTGGC TGACCCTGGC GGCCCTGGAC TGAGCGTCTG GAGGAGTCGT GGAGGCTCTT | 1560 |
| TCCCTTCTTT CTCCTCTGAG AGCTCGTTCT TCAGGCTCTT CCAGCTTGTC ATGTCGAGTG | 1620 |
| CCTGGCCACT GCTCAGGGTT GGAGGCTCAG TCCCTTTGCC CTGTCTGTTC CAGCTCTGGA | 1680 |
| GCTAACTCAG GGATCCCTGA TCAGGGTTAC ATAGGTTTGG TAAAATGAGT GCTGGAAATT | 1740 |
| AACTTTCTCC CAGTAGTCTT AGGTCATGCT CAGTGAACTT AAACTTTATC CAGATATGGT | 1800 |
| TTTCCTTCAG CCTTTCTATT CCCTTTCTAG CCAGTGAAAG ACCCGCTGCC CTTTGACCTC | 1860 |
| AGCCCCTCCA AGCCCCCAAG TTTAAAACGC CACCCCCTGC CGGCCCTGGA CTGAGCGTCT | 1920 |
| GGAGGAGTCG TGGAGGCTCT TTCCCTTCTT TCTCCTCTGA GAGCTCGTTC TTCAGGCTCT | 1980 |
| TCCAGCTTGT CATGTCGAGT GCCTGGCCAC TGCTCAGGGT TGGAGGCTCA GTCCCTTTGC | 2040 |
| CCTGTCTGTT CCAGCTCTGG AGCTAACTCA GGGATCCCTG ATCAGGGTTA CATAGGTTTG | 2100 |
| GTAAAATGAG TGCTGGAAAT TAACTTTCTC CCAGTAGTCT TAGGTCATGC TCAGTGAACT | 2160 |
| TAAACTTTAT CCAGATATGG TTTTCCTTCA GCCTTTCTAT TCCCTTTCTA GCCAGTGAAA | 2220 |
| GACCCGCTGC CCTTTGACCT CAGCCCCTCC AAGCCCCCAA GTTTAAAACG CCACCCCCTG | 2280 |
| CCACCAGAAA AAACAGAAAA AAAAAAAAAA AAAAAACTAA AACACCCATC TGGTCTGGGC | 2340 |
| ATCTTCCTTT CCTTTTTCAC TATGTATCCT GTTACTGGGC TTAAACAGCT TTCAGAGAAG | 2400 |
| AGATGTCATT TCTATTAAAT GCTCTTTCAG TAGCGAACTG AGTTCACACT TGACTAAGGA | 2460 |
| TATTTTCCGG ACTGTCTGTC ATCAGCATCC TTAGTGGGTT TCCCCATATT TAAATTGGTA | 2520 |
| GAGGCCAGGG ATGGTGGCTC ACACCTGTAA TCTCAGTACT TTGGGAGGCC AAGGTAGGTG | 2580 |
| GATTGCTTGA GCTCAGAAGA CCAGCCTGGG CAACCTGGTG AAACCCTGTC TCTACTAAAA | 2640 |
| ATTCAAGTTA GCTAGCTGGG CATGGTGATG CACTTCTGTA GTCCCAGCTA CTTGGAGAGG | 2700 |
| GGGTGGTGCT GGGGCAGCAG GATCGCTTGA ACCCAGGAGG TTGAGGTTGC AGTGAGCCAA | 2760 |
| GATGGTACCA GCCTAGGTGA CAAAGTGACA CCCTGTCTCA AAAAAGAAAC CAAACAAACA | 2820 |
| TAAAAAAAAA AAAAAAAAA | 2839 |
| Seq ID NO: 13 | |
| Primekey #: 450177 | |
| Coding sequence: 310..2037 | |
| 1 11 21 31 41 51 | |
| \| \| \| \| \| \| | |
| AGCGGAGGCG GCGGCGGCGG CGGCGGCGGC AGAGGGAGTT TCCGCTTTGC ACTCCACCCC | 60 |
| GGTAGCAGCT CCGCGGCAGG GACAGCTTCC TCCGGACGCT TGGCGGGCTT CGCTCTCGCC | 120 |
| TTACGACAGC CCGGTCGGAT CATGGGTTTG CCCAGGGGGC CGGAGGGCCA GGGTCTCCCG | 180 |
| GAGGTGGAAA CAAGAGAAGA TGAAGAACAA AATGTCAAGT TGACTGAAAT TCTGGAGCTC | 240 |
| TTGGTTGCAG CTGGGCATTT CAGGGCAAGA ATTAAAGGCT TATCACCCTT TGACAAGGTA | 300 |
| GTAGGAGGAA TGACTTGGTG TATCACCACT TGCAACTTTG ATGTAGATGT TGATTTGCTC | 360 |
| TTTCAAGAAA ACTCTACGAT AGGTCAAAAA ATAGCTCTGT CAGAAAAAAT TGTCTCGGTC | 420 |
| CTGCCAAGGA TGAAATGCCC ACACCAGCTG GAGCCCCACC AGATCCAGGG GATGGATTTT | 480 |
| ATTCACATAT TTCCTGTTGT TCAGTGGCTG GTGAAACGAG CTATAGAAAC AAAAGAAGAG | 540 |
| ATGGGTGACT ATATCCGCTC CTACTCTGTA TCCCAGTTCC AGAAGACTTA CAGTCTCCCT | 600 |
| GAGGATGATG ACTTCATAAA GAGAAAAGAA AAGGCCATCA AGACAGTTGT GGACCTCTCA | 660 |
| GAAGTGTACA AGCCCCGTCG GAAATACAAA CGCCACCAGG GAGCAGAGGA GCTACTTGAT | 720 |
| GAAGAATCTC GAATCCATGC TACACTTTTG GAATATGGCA GGAGATATGG ATTTAGCTGC | 780 |
| CAGAGCAAAA TGGAGAAGGC TGAGGACAAG AAAACGGCAC TTCCAGCAGG GCTGTCAGCT | 840 |
| ACAGAAAAAG CTGATGCCCA CGAGGAAGAT GAGCTTCGAG CAGCTGAAGA GCAGCGTATT | 900 |
| CAGTCGCTGA TGACCAAGAT GACCGCTATG GCAAATGAGG AGAGCCGTCT CACCGCAAGC | 960 |
| TCCGTGGGCC AGATTGTGGG ACTCTGCTCT GCTGAGATCA AGCAGATTGT GTCCGAGTAT | 1020 |
| GCAGAGAAGC AGTCTGAGCT ATCAGCTGAA GAAAGTCCAG AAAAATTAGG AACCTCCCAG | 1080 |
| CTACATCGCC GGAAAGTCAT TTCCTTGAAC AAACAGATTG CGCAAAAGAC CAAACATCTT | 1140 |
| GAAGAGCTGC GAGCAAGTCA CACCAGCCTA CAAGCCAGAT ATAATGAAGC CAAGAAAACG | 1200 |
| CTGACAGAGC TGAAGACTTA CAGTGAGAAA CTGGACAAAG AGCAAGCAGC CCTCGAGAAG | 1260 |
| ATAGAATCCA AAGCTGATCC AAGTATCCTA CAGAACCTGA GAGCACTTGT AGCCATGAAT | 1320 |
| GAAAATCTGA AAAGTCAAGA ACAGGAATTT AAAGCACATT GTCGAGAGGA GATGACACGA | 1380 |
| CTACAGCAAG AAATTGAAAA CCTGAAAGCT GAGAGAGCAC CACGTGGAGA TGAAAAGACC | 1440 |
| CTCTCCAGTG GAGAGCCGCC TGGTACCTTG ACCTCTGCAA TGACTCATGA CGAAGACCTA | 1500 |
| GACAGACGGT ATAATATGGA GAAAGAGAAA CTTTACAAGA TACGTTTACT ACAGGCTCGA | 1560 |
| AGAAATCGAG AAATAGCAAT TTTGCACCGC AAGATTGATG AAGTCCCTAG CCGTGCCGAG | 1620 |
| CTAATACAGT ATCAGAAGAG ATTTATTGAA CTCTACCGCC AGATTTCAGC AGTGCACAAA | 1680 |
| GAAACCAAGC AGTTCTTCAC TTTATATAAT ACCCTGGATG ATAAAAAGGT TTATTTGGAA | 1740 |
| AAAGAGATTA GTCTGCTGAA CTCAATTCAT GAGAACTTCT CACAGGCCAT GGCCTCCCCT | 1800 |
| GCTGCCCGGG ACCAGTTTTT ACGTCAGATG GAACAGATTG TGGAAGGAAT TAAGCAAAGT | 1860 |
| AGAATGAAGA TGGAAAAGAA AAAGCAAGAG AACAAAATGA GAAGAGACCA GTTGAACGAC | 1920 |
| CAGTACTTGG AGCTGTTAGA AAAGCAGAGG CTATACTTTA AGACTGTGAA AGAGTTCAAG | 1980 |
| GAGGAGGGCC GCAAGAACGA GATGCTGCTG TCCAAGGTGA AAGCGAAGGC CTCCTGAACA | 2040 |
| TCCCCAGCCG TGGCTGTATG TCATTGATTT TACTTTTAAG CACCGTATAT CACCTACAAG | 2100 |
| ATCATGAAAT GGTTCTGAAA GCGACAGTAG AGAGATGCAG TTGTGATGAT TTCAACAACC | 2160 |
| TGGATGTTTT CTTTCTCCTC TTTGCTTCCA TTCATCTCTG TTGGCTGCTG TTGATGGAGT | 2220 |
| CAGACAGTAA ACACGTGGCT TGGATAACAC CCATCATCCT ATGAAGAATA TAGGGAGTAC | 2280 |
| TTGTTCTCTG TTGATTCAAC TTTTATGTCT CCAGTAACAT TGCGCTTATG AAGGTACCTG | 2340 |
| TATTTGTATG GACTCTGAAT AAAGAAGAAT TCATTTGTTT AGCAAGTATT AGTTCAGCAA | 2400 |
| CCACTGAGAA ATAAGCACTG AGGAAGATTC AGAGACGTGT AAAACACAGT TCCTACTGCA | 2460 |
| CAAGTACCCA GCAGGTGGCC CAGGGAGGCA GATACAGCAC ACTTGACCGC AGAACTGGGC | 2520 |
| TATCCAAGAT GTTTTTCAGT AAACAGAAGG CATTTAGCTG AAATGATCAG CCCATGTAGT | 2580 |
| GTTGGTCACT TGGGCCTTTC ACCTGCCATG GTACCTTTTG TTCCCAGCTC CTCCAGGTGC | 2640 |
| CAGCCAGCAG GCTTGGTGGT GACAGCAACT GGAACGAAAG TTCAGTGTTG TTTTAATTTT | 2700 |
| TATACGTTAC TCAAGTTGAT TTCTCAGAAA ATTGAAAACA GACCTTGTGC TGAGGACACG | 2760 |
| TCAATAAAAA TTATACCTTC CCCTACAAAA AAAAAAAAAA AA | 2802 |
| Seq ID NO: 14 | |
| Primekey #: 407618 | |
| Coding sequence: 39..761 | |
| 1 11 21 31 41 51 | |
| \| \| \| \| \| \| | |
| GGAATTCCGT CGACGGCAGC GGCGGCGGCG GGTGGGAAAT GGCGGAGTAT CTGGCCTCCA | 60 |
| TCTTCGGCAC CGAGAAAGAC AAAGTCAACT GTTCATTTTA TTTCAAAATT GGAGCATGTC | 120 |
| GTCATGGAGA CAGGTGCTCT CGGTTGCACA ATAAACCGAC GTTTAGCCAG ACCATTGCCC | 180 |
| TCTTGAACAT TTACCGTAAC CCTCAAAACT CTTCCCAGTC TGCTGACGGT TTGCGCTGTG | 240 |
| CCGTGAGCGA TGTGGAGATG CAGGAACACT ATGATGAGTT TTTTGAGGAG GTTTTTACAG | 300 |
| AAATGGAGGA GAAGTATGGG GAAGTAGAGG AGATGAACGT CTGTGACAAC CTGGGAGACC | 360 |
| ACCTGGTGGG GAACGTGTAC GTCAAGTTTC GCCGTGAGGA AGATGCGGAA AAGGCTGTGA | 420 |
| TTGACTTGAA TAACCGTTGG TTTAATGGAC AGCCGATCCA CGCCGAGCTG TCACCCGTGA | 480 |
| CGGACTTCAG AGAAGCCTGC TGCCGTCAGT ATGAGATGGG AGAATGCACA CGAGGCGGCT | 540 |
| TCTGCAACTT CATGCATTTG AAGCCCATTT CCAGAGAGCT GCGGCGGGAG CTGTATGGCC | 600 |
| GCCGTCGCAA GAAGCATAGA TCAAGATCCC GATCCCGGGA GCGTCGTTCT CGGTCTAGAG | 660 |
| ACCGTGGTCG TGGCGGTGGC GGTGGCGGTG GTGGAGGTGG CGGCGGACGG GAGCGTGACA | 720 |
| GGAGGCGGTC GAGAGATCGT GAAAGATCTG GGCGATTCTG AGCCATGCCA TTTTTACCTT | 780 |
| ATGTCTGCTA GAAAGTGTTG TAGTTGATTG ACCAAACCAG TTCATAAGGG GAATTTTTTA | 840 |
| AAAAACAACA AAAAAAAAAC ATACAAAGAT GGGTTTCTGA ATAAAAATTT GTAGTGATAA | 900 |
| CAGT | 904 |
| Seq ID NO: 15 | |
| Primekey #: 435937 | |
| Coding sequence: 27..1721 | |
| 1 11 21 31 41 51 | |
| \| \| \| \| \| \| | |
| CGGGTGGTTG AGTGGAAGCG GTCGCCATGT CCGCGGGGAG CGCGACACAT CCTGGAGCTG | 60 |
| GCGGGCGCCG CAGCAAATGG GACCAACCAG CTCCAGCCCC ACTTCTCTTC CTCCCGCCAG | 120 |
| CGGCCCCAGG TGGGGAGGTC ACCAGCAGTG GGGGAAGTCC TGGGGGCACC ACAGCTGCTC | 180 |
| CTTCAGGAGC CTTGGATGCT GCTGCTGCTG TGGCTGCCAA GATTAATGCC ATGCTCATGG | 240 |
| CAAAAGGGAA GCTGAAACCA ACTCAGAATG CTTCTGAGAA GCTTCAGGCT CCTGGCAAAG | 300 |
| GCCTAACTAG CAATAAAAGC AAGGATGACC TGGTGGTAGC TGAAGTAGAA ATTAATGATG | 360 |
| TGCCTCTCAC ATGTAGGAAC TTGCTGACTC GAGGACAGAC TCAAGACGAG ATCAGCCGAC | 420 |
| TTAGTGGGGC TGCAGTATCA ACTCGAGGGA GGTTCATGAC AACTGAGGAA AAAGCCAAAG | 480 |
| TGGGACCAGG GGATCGTCCA TTATATCTTC ATGTTCAGGG CCAGACACGG GAATTAGTGG | 540 |
| ACAGAGCTGT AAACCGGATC AAAGAAATTA TCACCAATGG AGTGGTAAAA GCTGCCACAG | 600 |
| GAACAAGTCC AACTTTTAAT GGTGCAACAG TAACTGTCTA TCACCAGCCA GCACCCATCG | 660 |
| CTCAGTTGTC TCCAGCTGTT AGCCAGAAGC CTCCCTTCCA GTCAGGGATG CATTATGTTC | 720 |
| AAGATAAATT ATTTGTGGGT CTAGAACATG CTGTACCCAC TTTTAATGTC AAGGAGAAGG | 780 |
| TGGAAGGTCC AGGCTGCTCC TATTTGCAGC ACATTCAGAT TGAAACAGGT GCCAAAGTCT | 840 |
| TCCTGCGGGG CAAAGGTTCA GGCTGCATTG AGCCAGCATC TGGCCGAGAA GCTTTTGAAC | 900 |
| CTATGTATAT TTACATCAGT CACCCCAAAC CAGAAGGCCT GGCTGCTGCC AAGAAGCTTT | 960 |
| GTGAGAATCT TTTGCAAACA GTTCATGCTG AATACTCTAG ATTTGTGAAT CAGATTAATA | 1020 |
| CTGCTGTACC TTTACCAGGC TATACACAAC CCTCTGCTAT AAGTAGTGTC CCTCCTCAAC | 1080 |
| CACCATATTA TCCATCCAAT GGCTATCAGT CTGGTTACCC TGTTGTTCCC CCTCCTCAGC | 1140 |
| AGCCAGTTCA ACCTCCCTAC GGAGTACCAA GCATAGTGCC ACCAGCTGTT TCATTAGCAC | 1200 |
| CTGGAGTCTT GCCGGCATTA CCTACTGGAG TCCCACCTGT GCCAACACAA TACCCGATAA | 1260 |
| CACAAGTGCA GCCTCCAGCT AGCACTGGAC AGAGTCCGAT GGGTGGTCCT TTTATTCCTG | 1320 |
| CTGCTCCTGT CAAAACTGCC TTGCCTGCTG GCCCCCAGCC CCAGCCCCAG CCCCAGCCCC | 1380 |
| CACTCCCAAG TCAGCCCCAG GCACAGAAGA GACGATTCAC AGAGGAGCTA CCAGATGAAC | 1440 |
| GGGAATCTGG ACTGCTTGGA TACCAGCATG GACCCATTCA TATGACTAAT TTAGGTACAG | 1500 |
| GCTTCTCCAG TCAGAATGAG ATTGAAGGTG CAGGATCGAA GCCAGCAAGT TCCTCAGGCA | 1560 |
| AAGAGAGAGA GAGGGACAGG CAGTTGATGC CTCCACCAGC CTTTCCAGTG ACTGGAATAA | 1620 |
| AAACAGAGTC CGATGAAAGG AATGGGTCTG GGACCTTAAC AGGGAGCCAT GGTGAGTGTG | 1680 |
| ATATAGCTGG GGGAACAGGG GAGTGGCTAA GACTGGTCTA AAGCTATTAG TTTTCTCAGC | 1740 |
| CGGGCGCAGT GGCTCACGCC TGTAATCCCA GCACTTTGGG AGGCCGAGGT GGGCAGATCA | 1800 |
| CCTAAGGTCA GGAGTTCAAG ACCAGCTTGG CCAACATAGT GAAATCCCAT CTCTACTAAA | 1860 |
| AATACAAAAA CTAGCGGGCA TGGTGGTGGG CGCCTGTAAT TCCAGCTACT CAGGGGGTTG | 1920 |
| AGGCAGGAGA ATCGCTTCAA CCTGGGAGGC AGAGGTTGCA GTGAGCCAAG ATCAGACCAC | 1980 |
| TGCCCTCCAG CCTGGGCAAT AGAGCAAGAC TCCATCTCAT AAATAAATAA ATACATAAAT | 2040 |
| AAAGCTATTA ATTTTCTAAC CTGATGTTCA TTCAGGTGTT TAATCCAACC TCTATAATCT | 2100 |
| GTTGGCCAGT GAAAATACTT TTGGGCTGGG CACGGTGGCT CACGCCTGTA ATCCCAGCAC | 2160 |
| TTTGGGAGGC CAAGGTGGGC GGATAACCTG AGGTCAGGAG TTTGAGACCA GCGTGGCTAA | 2220 |
| CACGGTGAAA CCCCGTCTCT ACTAAAAATA GAAAAATTAA GCTGGGCATG GTGGTGCATG | 2280 |
| CCTGTAATTC CAGCGGCTTG GAAGGCTGAG GCAGGAGAAT CACTTGAACT TGGGAGGTGG | 2340 |
| AGGTTGCAGT GGGCCGAGAT CACACCACTG CATTCCAGCC TGGGCACTAG AGTGAGACTC | 2400 |
| TGTCTCAAAA AAAAAGAAAG AGAAAGAGAA AATAGTTTCT AAAAAATTGT ATACAGACAA | 2460 |
| CCTTTTATTT CCAACAAACG TGTGCCGAGA GAGAGAGAGA GAAAATAGTT TTAAAAAAAT | 2520 |
| TGTATACAGA CAACCTTTTG TTTCCAACCA ACGTGTATCT AGAAAAGAGT TAGTCGACTT | 2580 |
| ATTTTATACA TAGCATCAGT GAATAGTAAT GAGTGGTAGG TCATTTCAAA ATCCTGTTGC | 2640 |
| CTATATTATG TGAATACCAG GAGGTCATCT GATACGGACT TAATAAAGGT TGATTTTGCT | 2700 |
| TTATATTGGG AGCTGAGCCA CACCTCCCCT TATAACTCTA TTGGTCAGTA ATGGTCAGTT | 2760 |
| TGTGGCTGTT AGGAAAATGT TGCCTTTTAG CATTCCAGAA CTCTAAATCC TGTAGAGGTA | 2820 |
| CATGGGATAT TTTATTCTTT GCCTGTACTC ATAAAAATGA ACAGAAGAAA ATACGTTTTT | 2880 |
| TTCTTTTCTT AACTTCTTTT CTTTTAACTC TTTAAAAGGT GAAATATCAG CCCTCAAGAG | 2940 |
| ACTCACTTGC TAACTTTCCT TTTTTTCTTT TTTTTTCTTT TTTTTGTGTT TCTTTTTTCT | 3000 |
| TTCTCTGTTT TCTTACATGG TTCTGGTGGA TTCACATTTG CTGATGCTGG TGCTGTTTTT | 3060 |
| CGTGTGATCT TCAACGTTTT TGGGTGACCA TTGACCCTGT GACCTCAAAA TGGTGTCCAA | 3120 |
| CTAACCACTT AAAATTAACA TCTTTTTTTT AATTAACGAA TTTATGGTAT TTTTTTTTTT | 3180 |
| CCCTTGGCGG GGATGGGGTT GGGGTTGTTT TTTCTCTATT CTAGATTATC CAGCCAAGAA | 3240 |
| GATGAAAACT ACAGAGAAGG GATTTGGCTT GGTGGCTTAT GCTGCAGATT CATCTGATGA | 3300 |
| AGAGGAGGAA CATGGAGGTC ATAAAAATGC AAGTAGTTTT CCACAGGGCT GGAGTTTGGG | 3360 |
| ATACCAATAT CCTTCATCAC AACCACGAGC TAAACAACAG ATGCCATTCT GGATGGCTCC | 3420 |
| CTAGGAAACA GTGGAACAGA GTTTTGACCC TCAGTGACTC TTCTTAGCAA TAATGCATGC | 3480 |
| ATTTGATTTA ACAAGACTCT GGGGCCTGTG CTGGGAACCA TCTGGACCTT TGCAGAAGTT | 3540 |
| AGAGATTCAG TGCCCCCCTT TCTTAAAGGG GTTCCTTAAC AACCACAAAA ATCCTTATTT | 3600 |
| CTGCAGTGGC ATAGAATCTG TTAAAATTTA ATTAGAATCA CAAATTTATC TCAGAAGCTT | 3660 |
| TTTAACAGTT GGTGAAATGT GCTTGTCCAA CAAAGCATCC TAACAGGGTC GTTCCCATAC | 3720 |
| ACATTTGACC TGGTCAGCCT TTTCCAGGTG AATAGCCCCA GTTCTGACAT AAAGAAAGTT | 3780 |
| TTATTTGTAT TTTACTACTG TTTGGTCAAT TTTGATATAT AACTGGTTAC AAACAGAGCC | 3840 |
| TTACTATTTA TTAGTGGGGA AATGATTTTA AGACCGTCCT TTTCAGTATT TAATTCTGAC | 3900 |
| AGATCTGCAT CCCTGTTTTG TTTTGGATTA TTTCTGTTTT GGAAAATGCT GTCTCATTTA | 3960 |
| AAACTGTTGG ATATAGCTGG ATCCTGGATA GGAAAATGAA ATTATTTTTT CATTGTGTTT | 4020 |
| TTTAATTGGG GTGATCCAAA GCTGGCACCT TCAGGCACAT TGGTCTCATA GCCATTACTG | 4080 |
| TTTTTATTGC CCTTCTAAGA TCCTGTCTTC AGCTGGGTCA GAGAAAACTT CTTGACTAAA | 4140 |
| ACTGGTCAGA ACTCATCACA GAAATGAAAT ACAGTGGTCT CTCTCTCCCA GAACTGGTTG | 4200 |
| CAGCTAAAAC AGAGAGATCT GACTGCTGGC TATAGGATTT TGGACTTAAT GACTGAAATT | 4260 |
| GCAAATTGTC CTTTTTCTTG GCATTACAGA TTTTGCCAAA ATAACTTTTT GTATCAAATA | 4320 |
| TTGATGTGTG AAAGTGAAGG AGCTAGTCTG CTGAACCAGG AATAGTTTGA GATATTGAAC | 4380 |
| TGTCATTTTT GCACATTTGA ATACTTTGCA GGCTGGCTTT GTATAAACTT ATCCTCTGGT | 4440 |
| TTCCTATATG TTGTAAATAT TTAGACCATA ATTTCATTAT AAATAAATCT ATAAATATTC | 4500 |
| Seq ID NO: 16 | |
| Primekey #: 421221 | |
| Coding sequence: | |
| 1 11 21 31 41 51 | |
| \| \| \| \| \| \| | |
| TCGACTGCCA AAGCAATGAA GCTTGCGGCC GCGGCCACAG TCATGGCCTT TCCCCCTGGT | 60 |
| GCTCTTCATC CTTTACCAAA GAGACAAGCA CTTGAAAAAA GCAATGGTAC CAGCGCGGTC | 120 |
| TTTAACCCCA GCGTCTTGCA CTACCAGCAG GCTCTCACCA GCGCACAGTT GCAGCAACAC | 180 |
| GCCGCGTTCA TTCCAACAGG TATGTGCCCT TACTGCCCTA CGTCCTGTGC CCTTCTGGTC | 240 |
| ATGTGCTTTC TTCTCATTTC TCTAAGCTGT TTGGTGGCAT CTAGTTTGCT TTTGAAGGTA | 300 |
| TAATACAGTT TGAAATTCAT CGTTGTCCTA GCTATCTAAA TGTATTTACC TTACTTTGAA | 360 |
| TGATAGCTAA AGACTGTTAG GATTCTAAAG CCAAATATTT GATAGATTGA AGAGACAGAT | 420 |
| TTAACCCATG AGAAACAGCA GTTAGGGCTT TTGGTTTCTT GTATTTGCAC AAGCCCTGTA | 480 |
| AAATTGTTTA TGTAAATAAG ACCTTTTATG TGTGACAATT GAAATTTGTC CTTAACTCTG | 540 |
| AATGACCTAA AAATAGCAAT TCCAGTAAAT ACTAACCATT TTTTTCTATT TCTATTCAGA | 600 |
| GCACTAAAAC AATGAGGCTA TTCAAATTAA AGCAATTCTC TACTCATATT TTTATATTCA | 660 |
| TTCTATCTCT TTCTCCATCC TTCTCAACTT TCACCAAGTT CACAAGTATA TAGAGCTCTT | 720 |
| ATCCTCAGTG TCTAAGCCAA TGCCTGATAC TATTACGTAC GATGTGCATT AACTATGATT | 780 |
| CCACTAAAAG ATCCATTGTA ATAGTCATAG AATCTTAGAG TTTAAAGGAC TCTTAGTGAT | 840 |
| CTCCTCATCC AGCTGATTGT TTTACAGATG AGAAAACTGA GGCCCCCTAA ATGAGAAGTG | 900 |
| ACTTTCCAAG GTGCCACAAC TAATGAGAAA AAGAACTGAG TTTCCCTGTG ACCAAACCCA | 960 |
| TTTACATCAC ATTCTACCAC CTGGGCCCGC CTATATATAC ACATTCCACA GAGTTCTCCT | 1020 |
| GAAAAAAAAA AAAAGCAGAT AAAAGTGAAT TTTTAAATAA CTGACCCCAA AAAGTCAGAT | 1080 |
| AAAAGTAAAA AAACAAAAGT ATAAATCATG TCATCCCTCC CCCATTTGCA CCGACATCTC | 1140 |
| TAACCACAGA CACACACACG CACACCATAC GCAAAGATAG TCACCATAAT TGACCATGTT | 1200 |
| TTTCACCTTT TAGTCAATGT TAGAAGCAAG GGGTAACTTA AGTCCTGGTG GGAAGACCAT | 1260 |
| CCATTGAGTT CTTTGAAAGT CAACATTTTT CAGCCCACGA TAGTGAAATG AAAGTAAATA | 1320 |
| TAAATGAATA ACAATTCTAA CAAAAAGAGT TTTTTGATTC AAATCCATTA GTTTGAACTT | 1380 |
| TTCGAGCTTA TTATCCATTT CCTTAAATCC CATAGCTTAT CAGAGTTAAC ATCAGAGGGA | 1440 |
| GGTAAAATAT TTCTGTGATA TTCTTTGTAT AAAATCTACA CTTTGAAATG GATTAGTAAC | 1500 |
| CTGTGAACAA TACATATTTT AGTTAACATA TAAATTATGT GAGCAAAGTG GTTTTCAGTG | 1560 |
| TTTTTTTCTT ATTTTAGTTT TGAACCTGTC TTAAACTCAC AGACTTGTAG AAGAAATCTC | 1620 |
| TAATTCAGTA TTTATTAGGA GTTCACTTTT GCCCTATTAC AGCCTTAATT AGTGACATCC | 1680 |
| CAGTGCTGTT ACAGCATAGC AGTGTCTTAA TATGTAATCT AATTGAAATA ACACATTTGT | 1740 |
| AAAATAATTA CTAGAAGGTA AACTTACGTT AATGTCCTGT GTGGTTTCTA CAAAGTGTGT | 1800 |
| CATTGTAGAC CTCTTGGCCA CTAGATATTT TAAGATAAAA AAAAAAAAAA ATCGACGCGG | 1860 |
| CCGCGAATTT AGTAGTAGTA GTAGGC | 1886 |
| Seq ID NO: 17 | |
| Primekey #: 429766 | |
| Coding sequence: | |
| 1 11 21 31 41 51 | |
| \| \| \| \| \| \| | |
| CGGCACGAGG GCTGCTAAGA AGGCAGACAG CACCAAGCGC TAAATGAGAT GGGGCACCTG | 60 |
| GTGCTCTTCT GTGCTACTGG TAGGGGTGCA GCAGAGTGGT CAGTCTGGAC AGTAGCTGAC | 120 |
| ATCACGTGAC CCAACACACG CATTCCTGGC TACTTACCAA GGAGAATAGA AAGCAGGCAG | 180 |
| ATCTCTACAG CAGCTCTCTA CCTGATTGCA AAACAATGGA AATGCCCACA TGTCCACAAA | 240 |
| CAAGTGTGTG GTCTGCCTGT GCCATGAAGC ACAGTGTGGC TGAGCGTCAA GAGTCCCCAC | 300 |
| ACTCAAAGGA GGCAGCAGAT ACAGGGCTGC ACACTGTGTG ATTCCACACA TGTGACATTC | 360 |
| TGGACACGGA CATGCTGGAT GGCAAAACGA GCATCGGGCT GAGAGGACTG CTGAGAAGGG | 420 |
| GAACGGGGCT GCTGGGATGT GGGTTGATTG TAGCAGTAGC TCATGGAGAT GTGACCTCAA | 480 |
| AAGAGTGATT TTTACTATGT GCATACTATA CCTCCACAAA CTTGACTTTA AAAAAATAAA | 540 |
| ATATTCACAG AAAAAAACAA AAACAAATGT AAAACCATCA GACTACTTTA TCAGAGGTGT | 600 |
| TATTTTTAGA TAGAGGTCTT TGAACTCCAT CCTAGGAACA TTGTACCCAT GTCCTCCCAG | 660 |
| AACTGCATCT TGCACTGGGT GTCGGAAGAC AGCCCTGCAA GACCTGTATG CTCTGTACCA | 720 |
| TTCAGTGGTT TTTAAGGTTA ACTACCAGAA GTCATATCTG AGGCCTCCCA GAAGCATTAC | 780 |
| TCTAAGGAAA GTAGTTAAAT GTGGACAGTG ACAGCAGAAA CATTTACACA TTAAACCAGT | 840 |
| TTATAGAACA TGANNNNNNN NNNNNNNNAA AGAAGCTTGT CAGCTCAATG ACTTACGAGG | 900 |
| CGTGGGCCAT TAAAAAAAAA GGTCTGGAGT TTGGGAAGGA GAAAGGAATG GGGATGTGCA | 960 |
| GCTCAAGAGT GTGATTTTTA CTATGTGCAT AGTATACAGT GTGGAGACTT GACTTTAGGA | 1020 |
| AAGTAAAATA TTCACAGAAA AA | 1042 |
| Seq ID NO: 18 | |
| Primekey #: 450628 | |
| Coding sequence: | |
| 1 11 21 31 41 51 | |
| \| \| \| \| \| \| | |
| CAACTTCACG GACGCATTCA AGACCATGCT ATCATGGGAA ATCTGGTTAT GTTGTAATTT | 60 |
| TTAATATAAT TAAGGTAAAG CTTAAATGTG CTGTTACGTG ATTTCCTTTT AAAGTTTAAG | 120 |
| GTTATCTACC TTTGATATTC TCTGTAGATA TTAGTTGAAC ATAGTTCTCA CCAAAGTTAG | 180 |
| CTATCCAAAT TCAGGAAAAG CAAAACTATT TTTCCTTTTC TTTAAAAAGA AAACTTTGAT | 240 |
| TCATTTACTA GATTGTAAAC TTTTTTTTAA CTTCAAAAAT AATAAAAGGG TATGCAGGGA | 300 |
| AAAATCTTCC TCTCACCTGT CAGAGCTACT TTTTAAATAT GAAATAAGAG AAAACAAGTA | 360 |
| GCTGCTTATA AGGTGATGTG ATTACACTTA TAAAAGATGA ATTTAGAAAA CAACATTCAT | 420 |
| TGTCTAATTT AAATGGTCAA TAGAATCTTT ATTTTCTTTC TCCATAAGAC ATCCAGCTTC | 480 |
| ACAGCTTCAT GTGCTACCTA GAACTGATGA TGCCACAAAT CCTTAAATGT CCTAAATGGT | 540 |
| ACTGTTAAGT GAATCGTGCA ATTAGAATTT TCACCCAAAC AGAAGGGAAA CTGATTTTAG | 600 |
| ATGTGATTGG GCTTCTTGAG GACATTTCTG TGGTCTCGTT TTATTGTTTT TTTTTTTAGC | 660 |
| TTTGTTACTA TCTTAAATTC TTTGGTTATC AGCCTAGCAC TAAATGACCT TTAATTAAAA | 720 |
| AAAAAAAAAA AATCGTGCCG | 740 |
| Seq ID NO: 19 | |
| Primekey #: 450177 | |
| Coding sequence: | |
| 1 11 21 31 41 51 | |
| \| \| \| \| \| \| | |
| AATAGAATGA ATCCAATTTC TTGCCTTGGG TTACTGACTC TTTCAATTGT AACTAAGTAC | 60 |
| AATAGCAGTT AAGCTCAAGC TGTAATAGTA GAGCTCAGTG GAAGCTAAAC CAGGCACAGT | 120 |
| AACTGACACC ATGTAGGTTG ATTATATTTT GCATCTCCCT GCAAGTCTGT TTTATGTTAT | 180 |
| TTATAGCTTC CTATTCGTGT AGACACCAGC AGTAAACTGG GGAATATTTG TGGCAGGAAT | 240 |
| TTCTAAGAAC AACCTTTAGC ATCATCTCAG GCCCTGATCC ATTTCCTTTT CCACAAAATT | 300 |
| GTTTGAGATT ATATCGTATG TGTTACAGAA AGAATGTTTT TCTGTATGCT CGAAACTGTA | 360 |
| TACTAAAGTA AAATAATAAA GTTAACCAGA ATTATCCATG GGGAACAATT CCAATTAAAA | 420 |
| TAAAATGCCA GTATCTGGTA AAACCTGGTA GTAATGCTTT TTGTGGTGAT ATCCAGGTAA | 480 |
| TGATTAGATG CAGTAAACCC GGGTAGTAGG GAAGAAGAGA GATGTGGGGA CAAGCAGCCC | 540 |
| GAATACCTTG CTGGCATAGC AGCTGCCTAC CTGCACCCGG AGACCTGAGC AGATATTACT | 600 |
| AGGGTATTAT TTGACAGCCA GCTTAGCAGT CAAGAAGGAC ATTGATTTGG GGTAGCATGG | 660 |
| CAGACCACTT CATTGGGGCT GAAGACCTGC ATTTATTGAT CACTTACTAC ATGCCACGTA | 720 |
| TTTCGTTTAG GATATATATG TGTGCATGTG TATAATTTTA AAATATACCC CACGGTAGAG | 780 |
| GCAGAGCTGT TGGCAGTGAG CCGAGATCGC GCCACTGCAT TCCAGCCTGA GCGACAGAGC | 840 |
| GAGACTCTGT CTCAAAAAA | 859 |
| Seq ID NO: 20 | |
| Primekey #: 407618 | |
| Coding sequence: | |
| 1 11 21 31 41 51 | |
| \| \| \| \| \| \| | |
| TGCGCTACTT TTTTTGAGCC TGGGCGACAG ATTGAGACTC CGTCTCAAAA AAAAGAAAAA | 60 |
| AAAAAGAATG CTTTCATCAG CAAAACATTG TAACATTCCC TTTACTTGAG GGCGTCCACA | 120 |
| ATACCGTAAG GTTGCGTGAA CTGTCCTACT GAATCTTCAT GGTTGCTTGG ATTTTAATCA | 180 |
| CATCAGAAGA ATTTGAGAGC ATACCATGGC TGGCAGTCCA TAAAAGACTA GTTAGGAACA | 240 |
| TCAGCTTTTA ATCATCGACC CTGCTTTCAG GTTTCATTTT AAACTTATAG AAGAGGGGAA | 300 |
| GACATCAGTG TGCTTATTTG GCCTTTACTC TAAATCTTAA AAGGAAGAAA ATTTTAATAT | 360 |
| TTCTTAGTTT GAGCCCAGGT GCGGTGTCTC ACGCCTGTAA TCACAGCACT TTGGGAGGCC | 420 |
| AAGGCAGGCG GATCACTTGA GGTCAGGAGT TCAAGACCAG CCTGCAACGT GGTGAAACCC | 480 |
| TGTCTGTACT AAAAATTAAA AAAAAAAAAA AAAAAATTAG CCGGGCGTGG TGGCAGTCGC | 540 |
| CTGTAGTCCC AGCAACTCCA GAGGCTGAGA CAGGAGAATC GCTTGAACCC CAGAGGTGGA | 600 |
| GGTTGCAGTG AGCTGAGATG GTGCCACTGC ACTCCAGCCG TGGGCGACAG AGCCAGACTG | 660 |
| CATCTTGTGG GTGTAAAAAA AAAAATTTGT AGTTTGAGAG TCAACTTTTT CCTCACAGCT | 720 |
| TTCTGAAAAT GTGGCCCTTT GGATGCTGAT AAAAGCTGGT GGTGATTTTA ACACCTTAGT | 780 |
| AGCCAGAATC GAGACTGTCA TGGGGCACTT TTAAAATCTC ACCACGATTT GACTCCCATT | 840 |
| CACAAGGTAG CCATTGGGGC TCAGTCTCCC TGAATGCTCC TGCAAAAGTG CAGTCTGCCA | 900 |
| AGGTTTTCTC TAGAATAATC TCGGTGTGTG TTCACTGTAA CAGTTCTGAG TTACACCCAG | 960 |
| AGTTCATTCG GTTAACATTG TTCCTACCAG GCAAGACTTC TGGTGTTAGA AG | 1012 |
| Seq ID NO: 21 | |
| Primekey #: 435937 | |
| Coding sequence: | |
| 1 11 21 31 41 51 | |
| \| \| \| \| \| \| | |
| CATGATTACG GATTTTAATC CGCCTCATTA TAGGGAATTT GGCCCTCGAG GCCAAGAATT | 60 |
| CGGCCCCCAG GCACAGAAGA GACGATTCAC AGAGGAGCTA CCAGATGAAC GGGAATTTGG | 120 |
| ACTGCTTGGA TACCAGGTTA AATAAAATAC CCTGTTTTCC TATCTTCACC TTATTCTTCT | 180 |
| ACTATATTCT CCCTTTAAAA AAGATAAATT CACATCATTC TCCCAGTACT AGGATTTCTG | 240 |
| CTTTCTGGAA TTCATTTTGG TTAGGTTTTT TATCCTATTC AACAGACTCT TGAAAGCCTC | 300 |
| TGAGAGTTCT TACTTTCTTA TACATCTCAC TCAAAGCTCT TGATCTACCA GTATGTGGTT | 360 |
| TGTATTTAAA ACCTTGGCTT TCAGTGGTGC TCTCTCTTTT ACCCTCCACC TAAAAAAGAG | 420 |
| AGTGATATCT CCCTCCAGTC TCCCCACCCC TCAAGACTGC TAGAAAAGGA GTGATTCTGT | 480 |
| ACATGTAATT GTAAAGTTAG CCACTAAAGT TAAAAAGATT CTTAATTTGT AGTTTTGGTG | 540 |
| CAATTTTATC AGAAGTACCT TTCCATTTTG CCAGAATCCT TGAATCATTC TTTAAACCAA | 600 |
| AGCATTTTTT TATAGTTTCT AGCTAGGTTT ATAGAAACTA GTGGAGCTAT GGGCAGTCAG | 660 |
| TTAAAAACAG GCCATAGATA GCATAATGAA TTATAACACC CCTGTCCAAG TCCTATAGAG | 720 |
| AAAAAAAAAA AAAAA | 735 |
| PROTEIN SEQUENCES | |
| Seq ID NO: 22 | |
| Primekey #: 446619 | |
| 1 11 21 31 41 51 | |
| \| \| \| \| \| \| | |
| MRIAVICFCL LGITCAIPVK QADSGSSEEK QLYNKYPDAV ATWLNPDPSQ KQNLLAPQTL | 60 |
| PSKSNESHDH MDDMDDEDDD DHVDSQDSID SNDSDDVDDT DDSHQSDESH HSDESDELVT | 120 |
| DFPTDLPATE VFTPVVPTVD TYDGRGDSVV YGLRSKSKKF RRPDIQYPDA TDEDITSHME | 180 |
| SEELNGAYKA IPVAQDLNAP SDWDSRGKDS YETSQLDDQS AETHSHKQSR LYKRKANDES | 240 |
| NEHSDVIDSQ ELSKVSREFH SHEFHSHEDM LVVDPKSKEE DKHLKFRISH ELDSASSEVN | 300 |
| Seq ID NO: 23 | |
| Primekey #: 408199 | |
| 1 11 21 31 41 51 | |
| \| \| \| \| \| \| | |
| MQQRGAAGSR GCALFPLLGV LFFQGVYIVF SLEIRADAHV RGYVGEKIKL KCTFKSTSDV | 60 |
| TDKLTIDWTY RPPSSSHTVS IFHYQSFQYP TTAGTFRDRI SWVGNVYKGD ASISISNPTI | 120 |
| KDNGTFSCAV KNPPDVHHNI PMTELTVTER GFGTMLSSVA LLSILVFVPS AVVVALLLVR | 180 |
| MGRKAAGLKK RSRSGYKKSS IEVSDDTDQE EEEACMARLC VRCAECLDSD YEETY | 235 |
| Seq ID NO: 24 | |
| Primekey #: 421221 | |
| 1 11 21 31 41 51 | |
| \| \| \| \| \| \| | |
| MALNVAPVRD TKWLTLEVCR QFQRGTCSRS DEECKFAHPP KSCQVENGRV IACFDSLKGR | 60 |
| CSRENCKYLH PPTHLKTQLE INGRNNLIQQ KTAAAMLAQQ MQFMFPGTPL HPVPTFPVGP | 120 |
| AIGTNTAISF APYLAPVTPG VGLVPTEILP TTPVIVPGSP PVTVPGSTAT QKLLRTDKLE | 180 |
| VCREFQRGNC ARGETDCRFA HPADSTMIDT SDNTVTVCMD YIKGRCMREK CKYFHPPAHL | 240 |
| QAKIKAAQHQ ANQAAVAAQA AAAAATVMAF PPGALHPLPK RQALEKSNGT SAVFNPSVLH | 300 |
| YQQALTSAQL QQHAAFIPTG SVLCMTPATS IVPMMHSATS ATVSAATTPA TSVPFAATAT | 360 |
| ANQIILK | 367 |
| Seq ID NO: 25 | |
| Primekey #: 449491 | |
| 1 11 21 31 41 51 | |
| \| \| \| \| \| \| | |
| MASSPAVDVS CRRREKRRQL DARRSKCRIR LGGHMEQWCL LKERLGFSLH SQLAKFLLDR | 60 |
| YTSSGCVLCA GPEPLPPKGL QYLVLLSHAH SRECSLVPGL RGPGGQDGGL VWECSAGHTF | 120 |
| SWGPSLSPTP SEAPKPASLP HTTRRSWCSE ATSGQELADL ESEHDERTQE ARLPRRVGPP | 180 |
| PETFPPPGEE EGEEEEDNDE DEEEMLSDAS LWTYSSSPDD SEPDAPRLLP SPVTCTPKEG | 240 |
| ETPPAPAALS SPLAVPALSA SSLSSRAPPP AEVRVQPQLS RTPQAAQQTE ALASTGSQAQ | 300 |
| SAPTPAWDED TAQIGPKRIR KAAKRELMPC DFPGCGRIFS NRQYLNHHKK YQHIHQKSFS | 360 |
| CPEPACGKSF NFKKHLKEHM KLHSDTRDYI CEFCARSFRT SSNLVIHRRI HTGEKPLQCE | 420 |
| ICGFTCRQKA SLNWHQRKHA ETVAALRFPC EFCGKRFEKP DSVAAHRSKS HPALLLAPQE | 480 |
| SPSGPLEPCP SISAPGPLGS SEGSRPSASP QAPTLLPQQ | 519 |
| Seq ID NO: 26 | |
| Primekey #: 429766 | |
| 1 11 21 31 41 51 | |
| \| \| \| \| \| \| | |
| MAHGSQEAEA PGAVAGAAEV PREPPILPRI QEQFQKNPDS YNGAVRENYT WSQDYTDLEV | 60 |
| RVPVPKHVVK GKQVSVALSS SSIRVAMLEE NGERVLMEGK LTHKINTESS LWSLEPGKCV | 120 |
| LVNLSKVGEY WWNAILEGEE PIDIDKINKE RSMATVDEEE QAVLDRLTFD YHQKLQGKPQ | 180 |
| SHELKVHEML KKGWDAEGSP FRGQRFDPAM FNISPGAVQF | 220 |
| Seq ID NO: 27 | |
| Primekey #: 448518 | |
| 1 11 21 31 41 51 | |
| \| \| \| \| \| \| | |
| MLGAETEEKL FDAPLSISKR EQLEQQVGGV GQRWRQVQWP RALPELLSSQ GCWAPYSTHG | 60 |
| RCTQGLVGCP CRSLSPLTCP CLILQVPENY FYVPDLGQVP EIDVPSYLPD LPGIANDLMY | 120 |
| IADLGPGIAP SAPGTIPELP TFHTEVAEPL KTYKMGY | 157 |
| Seq ID NO: 28 | |
| Primekey #: 421999 | |
| 1 11 21 31 41 51 | |
| \| \| \| \| \| \| | |
| MQQRGAAGSR GCALFPLLGV LFFQGVYIVF SLEIRADAHV RGYVGEKIKL KCTFKSTSDV | 60 |
| TDKLTIDWTY RPPSSSHTVS IFHYQSFQYP TTAGTFRDRI SWVGNVYKGD ASISISNPTI | 120 |
| KDNGTFSCAV KNPPDVHHNI PMTELTVTER GFGTMLSSVA LLSILVFVPS AVVVALLLVR | 180 |
| MGRKAAGLKK RSRSGYKKSS IEVSDDTDQE EEEACMARL | 219 |
| Seq ID NO: 29 | |
| Primekey #: 450628 | |
| 1 11 21 31 41 51 | |
| \| \| \| \| \| \| | |
| MRGNLALVGV LISLAFLSLL PSGHPQPAGD DACSVQILVP GLKGDAGEKG DKGAPGRPGR | 60 |
| VGPTGEKGDM GDKGQKGSVG RHGKIGPIGS KGEKGDSGDI GPPGPNGEPG LPCECSQLRK | 120 |
| AIGEMDNQVS QLTSELKFIK NAVAGVRETE SKIYLLVKEE KRYADAQLSC QGRGGTLSMP | 180 |
| KDEAANGLMA AYLAQAGLAR VFIGINDLEK EGAFVYSDHS PMRTFNKWRS GEPNNAYDEE | 240 |
| DCVEMVASGG WNDVACHTTM YFMCEFDKEN M | 271 |
| Seq ID NO: 30 | |
| Primekey #: 450628 | |
| 1 11 21 31 41 51 | |
| \| \| \| \| \| \| | |
| MASLLKNGEP EAELHKETTG PGTAGPQSNT TSSLKGERKA IHTLQDVSTC ETKELLNVGV | 60 |
| SSLCAGPYQN TADTKENLSK EPLASFVSES FDTSVCGIAT EHVEIENSGE GLRAEAGSET | 120 |
| LGRDGEVGVN SDMHYELSGD SDLDLLGDCR NPRLDLEDSY TLRGSYTRKK DVPTDGYESS | 180 |
| LNFHNNNQED WGCSSRVPGM ETSLPPGHWT AAVKKEEKCV PPYVQIRDLH GILRTYANFS | 240 |
| ITKELKDTMR TSHGLRRHPS FSANCGLPSS WTSTWQVADD LTQNTLDLEY LRFAHKLKQT | 300 |
| IKNGDSQHSA SSANVFPKES PTQISIGAFP STKISEAPFL HPAPRSRSPL LVTAVESDPR | 360 |
| PQGQPRRGYT ASSLDISSSW RERCSHNRDL RNSQRNHTVS FHLNKLKYNS TVKESRNDIS | 420 |
| LILNEYAEFN KVMKNSNQFI FQDKELNDVS GEATAQEMYL PFPGRSASYE DIIIDVCTNL | 480 |
| HVKLRSVVKE ACKSTFLFYL VETEDKSFFV RTKNLLRKGG HTEIEPQHFC QAFHRENDTL | 540 |
| IIIIRNEDIS SHLHQIPSLL KLKHFPSVIF AGVDSPGDVL DHTYQELFPA GGFVISDDKI | 600 |
| LEAVTLVQLK EIIKILEKLN GNGRWKWLLH YRENKKLKED ERVDSTAHKK NIMLKSFQSA | 660 |
| NIIELLHYHQ CDSRSSTKAE ILKCLLNLQI QHIDARFAVL LTDKPTIPRE VFENSGILVT | 720 |
| DVNNFIENIE KIAAPFRSSY W | 741 |
| Seq ID NO: 31 | |
| Primekey #: 408806 | |
| 1 11 21 31 41 51 | |
| \| \| \| \| \| \| | |
| MPVRGDRGFP PRRELSGWLR APGMEELIWE QYTVTLQKDS KRGFGIAVSG GRDNPHFENG | 60 |
| ETSIVISDVL PGGPADGLLQ ENDRVVMVNG TPMEDVLHSF AVQQLRKSGK VAAIVVKRPR | 120 |
| KVQVAALQAS PPLDQDDRAF EVMDEFDGRS FRSGYSERSR LNSHGGRSRS WEDSPERGRP | 180 |
| HERARSRERD LSRDRSRGRS LERGLDQDHA RTRDRSRGRS LERGLDHDFG PSRDRDRDRS | 240 |
| RGRSIDQDYE RAYHRAYDPD YERAYSPEYR RGARHDARSR GPRSRSREHP HSRSPSPEPR | 300 |
| GRPGPIGVLL MKSRANEEYG LRLGSQIFVK EMTRTGLATK DGNLHEGDII LKINGTVTEN | 360 |
| MSLTDARKLI EKSRGKLQLV VLRDSQQTLI NIPSLNDSDS EIEDISEIES TRSFSPEERR | 420 |
| HQYSDYDYHS SSEKLKERPS SREDTPSRLS RMGATPTPFK STGDIAGTVV PETNKEPRYQ | 480 |
| EEPPAPQPKA APRTFLRPSP EDEAIYGPNT KMVRFKKGDS VGLRLAGGND VGIFVAGIQE | 540 |
| GTSAEQEGLQ EGDQILKVNT QDFRGLVRED AVLYLLEIPK GEMVTILAQS RADVYRDILA | 600 |
| CGRGDSFFIR SHFECEKETP QSLAFTRGEV FRVVDTLYDG KLGNWLAVRI GNELEKGLIP | 660 |
| NKSRAEQMAS VQNAQRDNAG DRADFWRMRG QRSGVKKNLR KSREDLTAVV SVSTKFPAYE | 720 |
| RVLLREAGFK RPVVLFGPIA DIAMEKLANE LPDWFQTAKT EPKDAGSEKS TGVVRLNTVR | 780 |
| QVIEQDKHAL LDVTPKAVDL LNYTQWFSIV ISFTPDSRQG VNTMRQRLDP TSNNSSRKLF | 840 |
| DHANKLKKTC AHLFTATINL NSANDSWFGS LKDTIQHQQG EAVWVSEGKM EGMDDDPEDR | 900 |
| MSYLTAMGAD YLSCDSRLIS DFEDTDGEGG AYTDNELDEP AEEPLVSSIT RSSEPVQHEE | 960 |
| SIRKPSPEPR AQMRRAASSD QLRDNSPPPA FKPEPSKAKT QNKEESYDFS KSYEYKSNPS | 1020 |
| AVAGNETPGA STKGYPPPVA AKPTFGRSIL KPSTPIPPQE GEEVGESSEE QDNAPKSVLG | 1080 |
| KVKIFGEDGS QGPGLQENAG APGSTECKDR NCPEAS | 1116 |
| Seq ID NO: 32 | |
| Primekey #: 408806 | |
| 1 11 21 31 41 51 | |
| \| \| \| \| \| \| | |
| MPVRGDRGFP PRRELSGWLR APGMEELIWE QYTVTLQKDS KRGFGIAVSG GRDNPHFENG | 60 |
| ETSIVISDVL PGGPADGLLQ ENDRVVMVNG TPMEDVLHSF AVQQLRKSGK VAAIVVKRPR | 120 |
| KVQVAALQAS PPLDQDDRAF EVMDEFDGRS FRSGYSERSR LNSHGGRSRS WEDSPERGRP | 180 |
| HERARSRERD LSRDRSRGRS LERGLDQDHA RTRDRSRGRS LERGLDHDFG PSRDRDRDRS | 240 |
| RGRSIDQDYE RAYHRAYDPD YERAYSPEYR RGARHDARSR GPRSRSREHP HSRSPSPEPR | 300 |
| GRPGPIGVLL MKSRANEEYG LRLGSQIFVK EMTRTGLATK DGNLHEGDII LKINGTVTEN | 360 |
| MSLTDARKLI EKSRGKLQLV VLRDSQQTLI NIPSLNDSDS EIEDISEIES TRSFSPEERR | 420 |
| HQYSDYDYHS SSEKLKERPS SREDTPSRLS RMGATPTPFK STGDIAGTVV PETNKEPRYQ | 480 |
| EEPPAPQPKA APRTFLRPSP EDEAIYGPNT KMVRFKKGDS VGLRLAGGND VGIFVAGIQE | 540 |
| GTSAEQEGLQ EGDQILKVNT QDFRGLVRED AVLYLLEIPK GEMVTILAQS RADVYRDILA | 600 |
| CGRGDSFFIR SHFECEKETP QSLAFTRGEV FRVVDTLYDG KLGNWLAVRI GNELEKGLIP | 660 |
| NKSRAEQMAS VQNAQRDNAG DRADFWRMRG QRSGVKKNLR KSREDLTAVV SVSTKFPAYE | 720 |
| RVLLREAGFK RPVVLFGPIA DIAMEKLANE LPDWFQTAKT EPKDAGSEKS TGVVRLNTVR | 780 |
| QVIEQDKHAL LDVTPKAVDL LNYTQWFPIV IFFNPDSRQG VKTMRQRLNP TSNKSSRKLF | 840 |
| DQANKLKKTC AHLFTATINL NSANDSWFGS LKDTIQHQQG EAVWVSEGKM EGMDDDPEDR | 900 |
| MSYLTAMGAD YLSCDSRLIS DFEDTDGEGG AYTDNELDEP AEEPLVSSIT RSSEPVQHEE | 960 |
| VRRGRPRAGT GEPGVFLALS WTAVCSGCCG RHS | 993 |
| Seq ID NO: 33 | |
| Primekey #: 407584 | |
| 1 11 21 31 41 51 | |
| \| \| \| \| \| \| | |
| MMWQKYAGSR RSMPLGARIL FHGVFYAGGF AIVYYLIQKF HSRALYYKLA VEQLQSHPEA | 60 |
| QEALGPPLNI HYLKLIDREN FVDIVDAKLK IPVSGSKSEG LLYVHSSRGG PFQRWHLDEV | 120 |
| FLELKDGQQI PVFKLSGENG DEVKKE | 146 |
| Seq ID NO: 34 | |
| Primekey #: 450177 | |
| 1 11 21 31 41 51 | |
| \| \| \| \| \| \| | |
| MTWCITTCNF DVDVDLLFQE NSTIGQKIAL SEKIVSVLPR MKCPHQLEPH QIQGMDFIHI | 60 |
| FPVVQWLVKR AIETKEEMGD YIRSYSVSQF QKTYSLPEDD DFIKRKEKAI KTVVDLSEVY | 120 |
| KPRRKYKRHQ GAEELLDEES RIHATLLEYG RRYGFSCQSK MEKAEDKKTA LPAGLSATEK | 180 |
| ADAHEEDELR AAEEQRIQSL MTKMTAMANE ESRLTASSVG QIVGLCSAEI KQIVSEYAEK | 240 |
| QSELSAEESP EKLGTSQLHR RKVISLNKQI AQKTKHLEEL RASHTSLQAR YNEAKKTLTE | 300 |
| LKTYSEKLDK EQAALEKIES KADPSILQNL RALVAMNENL KSQEQEFKAH CREEMTRLQQ | 360 |
| EIENLKAERA PRGDEKTLSS GEPPGTLTSA MTHDEDLDRR YNMEKEKLYK IRLLQARRNR | 420 |
| EIAILHRKID EVPSRAELIQ YQKRFIELYR QISAVHKETK QFFTLYNTLD DKKVYLEKEI | 480 |
| SLLNSIHENF SQAMASPAAR DQFLRQMEQI VEGIKQSRMK MEKKKQENKM RRDQLNDQYL | 540 |
| ELLEKQRLYF KTVKEFKEEG RKNEMLLSKV KAKAS | 575 |
| Seq ID NO: 35 | |
| Primekey #: 407618 | |
| 1 11 21 31 41 51 | |
| \| \| \| \| \| \| | |
| MAEYLASIFG TEKDKVNCSF YFKIGACRHG DRCSRLHNKP TFSQTIALLN IYRNPQNSSQ | 60 |
| SADGLRCAVS DVEMQEHYDE FFEEVFTEME EKYGEVEEMN VCDNLGDHLV GNVYVKFRRE | 120 |
| EDAEKAVIDL NNRWFNGQPI HAELSPVTDF REACCRQYEM GECTRGGFCN FMHLKPISRE | 180 |
| LRRELYGRRR KKHRSRSRSR ERRSRSRDRG RGGGGGGGGG GGGRERDRRR SRDRERSGRF | 240 |
| Seq ID NO: 36 | |
| Primekey #: 435937 | |
| 1 11 21 31 41 51 | |
| \| \| \| \| \| \| | |
| MSAGSATHPG AGGRRSKWDQ PAPAPLLFLP PAAPGGEVTS SGGSPGGTTA APSGALDAAA | 60 |
| AVAAKINAML MAKGKLKPTQ NASEKLQAPG KGLTSNKSKD DLVVAEVEIN DVPLTCRNLL | 120 |
| TRGQTQDEIS RLSGAAVSTR GRFMTTEEKA KVGPGDRPLY LHVQGQTREL VDPAVNRIKE | 180 |
| IITNGVVKAA TGTSPTFNGA TVTVYHQPAP IAQLSPAVSQ KPPFQSGMHY VQDKLFVGLE | 240 |
| HAVPTFNVKE KVEGPGCSYL QHIQIETGAK VFLRGKGSGC IEPASGREAF EPMYIYISHP | 300 |
| KPEGLAAAKK LCENLLQTVH AEYSRFVNQI NTAVPLPGYT QPSAISSVPP QPPYYPSNGY | 360 |
| QSGYPVVPPP QQPVQPPYGV PSIVPPAVSL APGVLPALPT GVPPVPTQYP ITQVQPPAST | 420 |
| GQSPMGGPFI PAAPVKTALP AGPQPQPQPQ PPLPSQPQAQ KRRFTEELPD ERESGLLGYQ | 480 |
| HGPIHMTNLG TGFSSQNEIE GAGSKPASSS GKERERDRQL MPPPAFPVTG IKTESDERNG | 540 |
| SGTLTGSHGE CDIAGGTGEW LRLV | 564 |

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

1. A method of diagnosing the health status of a biological sample, said method comprising the steps of:
a) generating a gene expression pattern of the biological sample, and
b) comparing the gene expression pattern of the biological sample with the reference sets of the Tables 1-6,
wherein a match between the gene expression pattern of the biological sample and one or more genes of the reference sets provides a diagnosis of the biological sample.

2. The method of claim 1, wherein the biological sample comprises cells obtained from a biopsy sample.

3. The method of claim 1, the biological sample is diagnosed as healthy tissue.

4. The method of claim 1, wherein the biological sample is diagnosed as having the potential to metastasize.

5. The method of claim 1, wherein the diagnosis identifies the tissue as having metastatic cancer.

7. The method of claim 1, wherein the comparison of the gene expression pattern of the biological sample and the reference sets is made with reference to at least one classifier genes from the Tables 1-6.

8. The method of claim 1, wherein the comparison of the gene expression pattern of the biological sample and the reference sets is made by comparing RNA expression profiles.

9. The method of claim 1, wherein the comparison of the gene expression pattern of the biological sample and the reference sets is made by comparing protein expression profiles.

10. The method of claim 10, wherein the protein expression profile is evaluated using antibodies.

11. A method for prognostic evaluation of the metastatic potential of colorectal cancer comprising the steps of
a) generating a gene expression pattern of a biological sample from the colorectal cancer, and
b) comparing the gene expression pattern of the biological sample with the reference sets of the Tables 1-6,
wherein a match between the gene expression pattern of the biological sample and one or more reference sets provides a prognosis evaluation of the metastatic potential of the colorectal cancer.

12. The method of claim 12, wherein a match between the gene expression pattern of the biological sample and the reference set representing colon cancer metastasis or Duke's stage D colorectal cancer is indicative of poor prognosis.

13. A method for evaluating the progress of a treatment regimen for metastatic colorectal cancer comprising the steps of:
a) generating a first gene expression pattern of a first biological sample from a patient,
b) comparing the first gene expression pattern of the first biological sample with the reference sets of the Tables 1-6,
c) obtaining a match between the first gene expression pattern of the first biological sample and one or more reference sets of the Tables 1-6, thereby providing an initial diagnosis of metastatic colorectal cancer,
d) administering to the patient a therapeutically effective amount of a compound that modulates the metastatic colorectal cancer,
e) generating a second gene expression profile of a second biological sample from the patient,
f) comparing the second gene expression pattern of the second biological sample with the reference sets of the Tables 1-6,
g) obtaining a match between the second gene expression pattern of the second biological sample and one or more reference sets of the Tables 1-6,
h) comparing the match between the first gene expression pattern of the first biological sample and the match between the second gene expression pattern of the second biological sample,
wherein the comparison indicates the progress of the treatment for metastatic colorectal cancer.

14. A method for evaluating the efficacy of drug candidates for use in the treatment of metastatic colorectal cancer comprising the steps of;
a) contacting a cell or tissue culture that has a gene expression profile indicative of metastatic colorectal cancer with an effective amount of a test compound,
b) generating a gene expression profile of the contacted cell or tissue culture,
c) comparing the gene expression pattern of the contacted cell culture with the defined sets of genes of the Tables 1-6,
d) obtaining a match between the gene expression pattern of the contacted cell culture and one or more reference sets of the Tables 1-6, thereby determining the efficacy of the drug for the treatment of metastatic colorectal cancer.

15. A kit for diagnosing the health status of a biological sample said kit comprising:
a) nucleic acid probes that specifically bind to nucleotide sequences from reference sets of the Tables 1-6, and
b) means of labeling nucleic acids.

17. The kit of claim 15, wherein the nucleic acid probes identify metastatic cancer derived from a primary tumor in an organ selected from the group consisting of heart, lung, pancreas, breast, prostate, and colon.

18. A kit for diagnosing the health status of a biological sample said kit comprising:
a) antibodies or ligands that specifically bind to polypeptides encoded by a genes of the reference sets of the Tables 1-6, and
c) means of labeling the antibodies or ligands that specifically bind to polypeptides encoded by genes of the reference sets of the Tables 1-6.

19. The kit of claim 17, wherein the antibodies or ligands identify metastatic cancer derived from a primary tumor in an organ selected from the group consisting of heart, lung, pancreas, breast, prostate, and colon.

20. A method for selecting patients for therapy of colon cancer based on the steps of:
a) generating a gene expression pattern of a biological sample from the patient, and
b) comparing the gene expression pattern of the biological sample with the reference sets of the Tables 1-6,
wherein a match between the gene expression pattern of the biological sample and one or more genes from the reference sets provides an evaluation of the metastatic potential of the colorectal cancer and thereby determines whether a patient will be selected for therapy.


SEQUENCE LISTING (36 sequences)

SEQ ID NO: 1 - DNA, 1524 bp, Homo Sapiens
gcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaa

SEQ ID NO: 2 - DNA, 3932 bp, Homo Sapiens
gtgcaagcatctgaagagctgccgggatgcagcagagaggagcagctggaagccgtggctgcgctctcttccctctgctgggcgtcctgttcttccagggtgtttatatcgtcttttccttggagattcgtgcagatgcccatgtccgaggttatgttggagaaaagatcaagttgaaatgcactttcaagtcaacttcagatgtcactgacaaacttactatagactggacatatcgccctcccagcagcagccacacagtatcaatatttcattatcagtctttccagtacccaaccacagcaggcacatttcgggatcggatttcctgggttggaaatgtatacaaaggggatgcatctataagtataagcaaccctaccataaaggacaatgggacattcagctgtgctgtgaagaatcccccagatgtgcatcataatattcccatgacagagctaacagtcacagaaaggggttttggcaccatgctttcctctgtggcccttctttccatccttgtctttgtgccctcagccgtggtggttgctctgctgctggtgagaatggggaggaaggctgctgggctgaagaagaggagcaggtctggctataagaagtcatctattgaggtttccgatgacactgatcaggaggaggaagaggcgtgtatggcgaggctttgtgtccgttgcgctgagtgcctggattcagactatgaagagacatattgatgaaagtctgtatgacacaagaagagtcacctaaagacaggaaacatcccattccactggcagctaaagcctgtcagagaaagtggagctggcctggaccatagcgatggacaatcctggagatcatcagtaaagactttaggaaccacttatttattgaataaatgttcttgttgtatttataaactgttcaggaactctcataagagactcatgacttcccctttcaatgaattatgctgtaattgaatgaagaaattcttttcctgagcaaaaagatactttttgattcatctttgctctggaatgtattacatgttttcttccaactgtttgaaggagaattttgaatgtttgccacaccgctgatacccaaataattttttaaatgaagtggagcttgtggcttcctgatgtgtcaccagacaaaatattcgcttgggatatgtattctttgttttttgctccatgtacactttcagctgtgagttagtatagggcgtatacttaccggtttaatgacctcaacctcagttgtgtttggataacttagggtgtatacccttagtttccttagagttggtaggatcaagtcattggtttgctttgactgggtttttaaagtattaagtacagtgtcatcaatttacagttaaggaaaggaatcgtgaagtagaaaaattattttctttagtcttgctggtacaatttgggctaaggagtctttgttattttctgtcttgcttttttttttttttttttttttttgaggcagagtctcactctgtcgccaggctggagtgcagtggtgtgatcttggctcactgcaacctctgcctcctgggttcaagcgattcttgtgcctcagcctctcgagtagctgggattacaggcatgcgccaccacacccagctaatttttgtgtttttagtagagacggggtttcaccattttggccaggatggtctcaatcccctgacctcgtgatccacctgcctcggcctcccaaagtgttgggattacaggcatgagccactgtgcttggcctgttattttattttcttataactacaacttttcttcttgaattttcaggtcagaggcaagaaaaactctttacaggtttttagtggggggcttatggagtatttcaggagttctttgcaaattaaatcatcttttcacttgtattgtttttcaaaactttgttgatttctaaaatgtgccaactgtgagtaaactatggtatttgcaagtggtttttacataatatttgagatgaggaagtgagattgtgcatgacatacttctcctttgtattctctcagtgccttacagcaggttactccattctgctatgacaacttgtttcaaatgttaatttacataggattttttataagccattaaggcatatgtatagtatatcagtaaagatggatggtgcatatataaatagtcttctgtaatagtgattggatttacttctcaattatgagagacaaaaattatcccctcacctgtctctattctttcaacaggttgatcccttttcatgatttttcattaggtggttcaggaagtttccatattacagcgcttcagactgtatatgttagtttaaaaatcacttttctctctctcaacttctttcttttttttttgaagacttaatttaaaaaatttgggttgttagatccgtatcatagatttggcctagcctcttctgttaacctagtccacagatgagcgaatctggttagttgaaggacattgtgatttgactctggtcacgcgaggaagtagaagggcaaagacaggaccggcagtttacatttccagtggttaaacctcacggtactttgggactgcttgttaacttttgtggttgtctgaggccaatctaacgtgaccatttctgacacctcaacagagagaggaaagcaacttgagcaatgagagtaaataacttgggctctcagagatttgaagatagagatctcattgtgagggggactattttgcaggtcctcatttctccaagaaagagatggtgttacaggaacccactgaaagccatatcccattaaatgaggaactaattttggctgggccttcttgtaatgtcctcgcaggtgtgttgtgaagattaatgcagggtagtatgtttgtagattgacacctagtctaaacttgaggtaattggtgctctgtgaatactcagtcgtgttcttttatagccttaatcatgatttgaactagtcccttgctttttaaatgactgaatgaagtccttcgtggtaagggagtacgttgataacttagtttactatatgggtttgtggtcgcatcccagtcatcagctgctatcattttccttcttcatcccttatactgagatttgggttacagctttttattcttcgaaggatcacaaagcagtgtacagacacctgccttctttaaggatgaaaggaagataaagtggtctttttttgtttacttatttgtttcacctcttgtttgagtaacttctaaggtgctattctctctctctttttgctacctcatgagctcttgtcacagccatggaaaccagcctcgtttagaaagggaacttagttcagaaggggttaaaagccttccagaatttttctttagctgctgaagtttttacatgtggttacatgactttaagttttatgcattacgctcttaattctattacaaaatgtggactcaccaattgctttgtgttttccatgtgacctgttacttcaggctacttggggaacatcttagtcctctgtagctcctgaacccagcactggtgcttcaagagagaaggtagcacgtctttgttcaaaacaaaacaaaacgacacttctggaggccacatcctgaatatgaatgttctactaagtcactcagttatggttctaaagggaaactgtaagaagacccacaaggagtggaccaagactattatttaattgcacaacttgaaactttgctgccagaagaggcagctccattcctttgactccagtgttgggctgttaactgctgcacctcattgcctttttttgtttttgtttttgttttgtaggagggtaggcactgttgggccatatgcacaaatattgtaactcttggtatctttactgcatcatagtcaataaacttctttgtaccctt

SEQ ID NO: 3 - DNA, 4665 bp, Homo Sapiens
tgaaggtaaaattttccagatacggcagacggctttcagagtacaataaacagggaatgagaactatttacatggaagtttctttctcatgatgcggtggagaagcctcggccacttggttctgccagatgttcctggggttactgtaaatgggaaggacaggcagagctaaacaaggtttatcatttaaaagtgcctgtgtgaagtcacttttgctggaaaactgcagcttgggagctttctttgtattcacatcccactcttctgtcaagtacactttaccctgaccttatgagtggatgaagatacctcagttgtctgactttgccaattgcttaatttcagaatttaaaaaggggaaagaaaaacatcctgctaaaatatgaacatctgagtgtcttattttccaacatcgtcaatagctgtgagcgtcagcattaaatattctcccaaggagtgccatgatattgaagtcactttattaataacagctgtatctgcaaaacagtcaagagactcggacgttgaaagccagagatgacactgagcatgcttttattgcggcctaccatctttaagtgggacatattgattgatgagtgattgcctgtccatacactctctcatcatcctgttccttggattggacttcactaagcaatttatcactcaccttcagacttacatgtgggagttttcacaacagtagttttggaatcattagaacttggattgatttcatcatttaacagaaacaaacagcccaaattactttatcaccatggctttgaacgttgccccagtcagagatacaaaatggctgacattagaagtctgcagacagtttcaaagaggaacatgctcacgctctgatgaagaatgcaaatttgctcatccccccaaaagttgtcaggttgaaaatggaagagtaattgcctgctttgattccctaaagggccgttgttcgagagagaactgcaagtatcttcaccctccgacacacttaaaaactcaactagaaattaatggaaggaacaatttgattcagcaaaaaactgcagcagcaatgcttgcccagcagatgcaatttatgtttccaggaacaccacttcatccagtgcccactttccctgtaggtcccgcgatagggacaaatacggctattagctttgctccttacctagcacctgtaacccctggagttgggttggtcccaacggaaattctgcccaccacgcctgttattgttcccggaagtccaccggtcactgtcccgggctcaactgcaactcagaaacttctcaggactgacaaactggaggtatgcagggagttccagcgaggaaactgtgcccggggagagaccgactgccgctttgcacaccccgcagacagcaccatgatcgacacaagtgacaacaccgtaaccgtttgtatggattacataaaggggcgttgcatgagggagaaatgcaaatattttcaccctcctgcacacttgcaggccaaaatcaaagctgcgcagcaccaagccaaccaagctgcggtggccgcccaggcagccgcggccgcggccacagtcatggcctttccccctggtgctcttcatcctttaccaaagagacaagcacttgaaaaaagcaatggtaccagcgcggtctttaaccccagcgtcttgcactaccagcaggctctcaccagcgcacagttgcagcaacacgccgcgttcattccaacagggtcagttttgtgcatgacacccgctaccagtattgtacccatgatgcacagcgctacgtccgccactgtctctgcagcaacaactcctgcaacaagtgtccccttcgcagcaacagccacagccaatcagataattctgaaataatcagcagaaacggaatggaatgccaagaatctgcattgagaataactaaacattgttactgtacatactatcctgtttcctcctcaatagaattgccacaaactgcatgctaaataaagatgtagttcttctggacagaccacaactctaagaagctagtgctgctatctcatatatgagtattaaatatggtatgcttagtatattccaacctaagatagttaactacctgagaccagctgtgatgtttaaagacataaaggataaagtttacttttaaagggtttctaaacatagtttctgtcctaggaatattgtcttatctccataactatagctgatgcagaaagtccagccagtttactcatttcgattcagaatatttcaaatttagcaataaacaattagcattagttaaaaaagaaacatattccaagggcaggttcgattctagctctaattactgtcatgtcatttacccactggatcaaagggtatgtttcacttcttgacaatataaatgctgcagcaaagatgagaggtgaagtaaaaccgatacctgtcctgcaggtctaaaatttgaatggaaattcaagcacaagtactggggacacatcaaagtgtggtgtttggtttgcctggagatgccacgttgaatcatgtgattctagattaacattaaatagattgaaaaagaaactttgcacggtatgagcttcataccccaccaaacaaagtcttgaaggtattattttacaagtatatttttaaagttgttttataagagagactttgtagaagtgcctagattttgccagacttcatccagcttgacaagattgagaggcccatgccaacagtctaatctaagagattagtctttcaaactcaccatccagttgcctgttacagaataactcttcttaactaaaaacctagtcaaacaaggaagctgtaggtgaggagatctgtataatattctaatttaagtaagtttgagtttagtcactgcaaatttgactgtgactttaatctaaattactatgtaaacaaaaagtagatagtttcactttttaaaaaatccattactgttttgcatttcaaaagttggattaaagggttgtaactgactacagcatggaaaaaaatagttcttttaattctttcaccttaaagcatattttatgtctcaaaagtataaaaaactttaatacaagtacatacatattatatatacacatacatatatatactatatatggatgaaacatattttaatgttgtttacttttttaaatacttggttgatcttcaaggtaatagcgatacaattaaattttgttcagaaagtttgttttaaagtttattttaagcactatcgtaccaaatatttcatatttcacattttatatgttgcacatagcctatacagtacctacatagtttttaaattattgtttaaaaaacaaaacagctgttataaatgaatattatgtgtaattgtttcaaacatccattttctttgtgaacatattagtgattgaagtattttgacttttgagattgaatgtaaaatattttaaatttgggatcatcgcctgttctgaaaactagatgcaccaaccgtatcattatttgtttgaggaaaaaaagaaatctgcattttaattcatgttggtcaaagtcgaattactatctatttatcttatatcgtagatctgataaccctatctaaaagaaagtcacacgctaaatgtattcttacatagtgcttgtatcgttgcatttgttttaatttgtggaaaagtattgtatctaacttgtattactttggtagtttcatctttatgtattattgatatttgtaattttctcaactataacaatgtagttacgctacaacttgcctaaaacattcaaacttgttttcttttttctgtttttttctttgttaattcatttaaactcattgaaaacatagtatacattactaaaaggtaaattatgggaatcactgaaatatttttgtagattaattgttgtaacattgtctttcttttttttcttttgtttcatgattttgatttttaaaattattagcacacaactattttcagccctttaataatggagcatcaaaaacatcacctgtaaccccaagcaaatatagaagactgtattttttactatgatatccattttccagaattgtgattacaatatgcaaagagtcataaatatgccatttacaataaggaggaggcaaggcaaatgcatagatgtacaaatatatgtacaacagattttgctttttatttatttataatgtaattttatagaataattctgggatttgagaggatctaaaactatttttctgtataaatattatttgccaaaagtttgtttatattcagaagtctgactatgatgaataaatcttaaatgctttgtttaattaaaaaacaaaaatcaccaatatccaagacatgaagatatcagttcaacaaatactgtagttaagagactaactctccacttgtatgggaactacatttcactcttggttttcaggatataacagcacttcaccgaaatattctttcagccataccactggtaacatttctactaaatctttctgtaacacttaaagaattccctcattcattaccttacagtgtaaacaggagtctaatttgtatcaatactatgttttggttgtaatattcagttcactcacccaatgtacaaccaatgaaataaaagaagcatttaaa

SEQ ID NO: 4 - DNA, 1980 bp, Homo Sapiens
agcagccgacgccgagaggcaccgtttcttcttaaaagagaaacgctgcgcgcgcgaggtgggcccctgtcttccagcagctccgggcctgctcgctaggcccgggaggcgcaggcgcaggcgcagtgggggtgagggcgcgtgggggcgcacagcctctggtgcacatggcttcctccccggcggtggacgtgtcctgcaggcggcgggagaagcggcggcagctggacgcgcgccgcagcaagtgccgcatccgcctgggcggccacatggagcagtggtgcctcctcaaggagcggctgggcttctccctgcactcgcagctcgccaagttcctgttggaccggtacacttcttcaggctgtgtcctctgtgcaggtcctgagcctttgcctccaaaaggtctgcagtatctggtgctcttgtctcatgcccacagccgagagtgcagcctggtgcccgggcttcgggggcctggcggccaagatggggggcttgtgtgggagtgctcagcaggccataccttctcctggggaccctctttgagccctacaccttcagaggcacccaagccagcctcccttccacatactactcggagaagttggtgttccgaggccacgagtgggcaggagcttgcagatttggaatctgagcatgatgagaggactcaagaggccaggttgcccaggagggtgggacccccaccagagaccttcccacctccaggagaggaagagggtgaggaagaagaggacaatgatgaggatgaagaggagatgctcagtgatgccagcttatggacctacagctcctccccagatgatagtgagcctgatgcccccagactactgccttcccctgtcacctgcacacctaaagagggggagacaccaccagcccctgcagcactctccagtcctcttgctgtgccggccttgtcagcatcctcattgagttccagagctcctccacctgcagaagtcagggtgcagccacagctcagcaggacccctcaagcggcccagcagactgaggccctggccagcactgggagtcaggcccagtctgctccaaccccggcctgggatgaggacactgcacaaattggccccaagagaattaggaaagctgccaaaagagagctgatgccttgtgacttccctggctgtggaaggatcttctccaaccggcagtatttgaatcaccacaaaaagtaccagcacatccaccagaagtctttctcctgcccagagccagcctgtgggaagtctttcaactttaagaaacacctgaaggagcacatgaagctgcacagtgacacccgggactacatctgtgagttctgcgcccggtctttccgcactagcagcaaccttgtcatccacagacgtatccacactggagaaaaacccctgcagtgtgagatatgcgggtttacctgccgccagaaggcttccctgaactggcaccagcgcaagcatgcagagacggtggctgccttgcgcttcccctgtgaattctgcggcaagcgctttgagaagccagacagtgttgcagcccaccgtagcaaaagtcacccagccctgcttctagcccctcaagagtcacccagtggtcccctagagccctgtcccagcatctctgcccctgggcctctgggatccagcgaggggtccaggccctctgcatctcctcaggctccaaccctgcttcctcagcaatgagctctcctccagctttggctttgggaagccagactccagggactgaaaaggagcaacaaggagagggtctgcttgagaaatgccagatgcttggtccccaggaactaaggcgacagagtgcagggtgggggcaagactgggctgtaggggagctggactactttagtcttcctaaaggacaaaataaacagtattttatgcaggaaaaaaaaaaaaaaaaaaaaaaaaaaaaaaaaaaaaaaaaaaaaaaaaa

SEQ ID NO: 5 - DNA, 3584 bp, Homo Sapiens
cggacgcgtgggctgaggcggcgctgtgtgtgtgaagcgtacctagggcgggaggcgacatggagacaggggcggccgagctgtatgaccaggcccttttgggcatcctgcagcacgtgggcaacgtccaggatttcctgcgcgttctctttggcttcctctaccgcaagacagacttctatcgcttgctgcgccacccatcggaccgcatgggcttcccgcccggggccgcgcaggccttggtgctgcaggtattcaaaacctttgaccacatggcccgtcaggatgatgagaagagaaggcaggaacttgaagagaaaatcagaagaaaggaagaggaagaggccaagactgtgtcagctgctgcagctgagaaggagccagtcccagttccagtccaggaaatagagattgactccaccacagaattggatgggcatcaggaagtagagaaagtgcagcctccaggccctgtgaaggaaatggcccatggttcacaggaggcagaagctccaggagcagttgctggtgctgctgaagtccctagggaaccaccaattcttcccaggattcaggagcagttccagaaaaatcccgacagttacaatggtgctgtccgagagaactacacctggtcacaggactatactgacctggaggtcagggtgccagtacccaagcacgtggtgaagggaaagcaggtctcagtggcccttagcagcagctccattcgtgtggccatgctggaggaaaatggggagcgcgtcctcatggaagggaagctcacccacaagatcaacactgagagttctctctggagtctcgagcccgggaagtgcgttttggtgaacctgagcaaggtgggcgagtattggtggaacgccatcctggagggagaagagcccatcgacattgacaagatcaacaaggagcgctccatggccaccgtggatgaggaggaacaggcggtgttggacaggcttacctttgactaccaccagaagctgcagggcaagccacagagccatgagctgaaagtccatgagatgctgaagaaggggtgggatgctgaaggttctcccttccgaggccagcgattcgaccctgccatgttcaacatctccccgggggctgtgcagttttaatgaccagaaggaaaggaaaccctcgccggtggggaggcagagccttatcctcggctgcccttcttggctccctgcattccagggacttgctcgtcttgtttacccctagccatcctttctttcaagggtgaaccaggccttccaccctgaccttgcatctccagactgttccagagaaggtgcggggccagctgctatgtggtggccgctgtggctgacactgagtgaaggtgtttgaaatgcaggagaggatatcccagcaaattgggatcacatgcttttgtctccacagcaaccagccactgcaggcagcatgtctttcctcccctgctctctgcttgctgttgttttgacgctattctgcttgcatgtcttctggttgggatgtggagttgttgctggactctcaggcgaagctgaagtcattgaagtgtgtgaagctctgtgcttgcatgagggcaagcaaggaatggctgtgcctgaggctgctctgggaaactccttgccccttgacctcttttgagagcattcacgtggtcttcttgctcatccccttataaatgtgctttgcctgcctcagcctcatggtcagagcagtggagactggagccctgtttgcacgttctagttgttcggagaaagcctaggttctgggctcaggtccagatgcagcggggattctgttctctgactgtggcgaccttgctttggttcttgttgaagtgaaccaagcccggccaccacgcatggcatgctgtgcttggctccccataagacgtcctctttgggtgcacggtgtcaaagtgtgggcaggagtggagagctggtgccctcaggaggagaccacagcatgtccatcagctcagcagagctcgacagccacaagtcctgagaagctttgaccttgaagggcttctgggagaggaggaatttctgcatggggcgtgaaggcacactgtcccaccacaactgaaccagaagagagtgaagactcccctcttcccatcctctgtgccaggtgccagactgtgctccttggaacttatggcccaatcttacctgttctccagggactggtcactgcctcaggacccccaagcctatgccctgagccatggctgctgactgactccagccaaggtgcaaagacgagattatgagacaggtcctcaggcctgtgttccaagtactcacaggggctctgggtgcccatcgccgggagtatggttcagctgccaccggcactgtccatttgcctgtctgtcaagctcagagcatggataagccacacagcagggcagtgcaccctggcaccatgcacggccagcaagaatcaaggcccgcagatgctaagagggcctattgtcaggggaaggtccccgctcctgcacactctctatggatacttgggttgtgggggctctcttggagagtaagtttgtggtttgtttctggtttacagtggtggctgacaccccttgtaagaaagcattcctgggaagtcttctgtgggtccaaacatgttgctccgatcatcacaggagagcaaaaggccctagataccccctttggaatgtgagagtcttgttgtctgatatttgccactgagctggtgaagcccctctaaagagatctcgaccctggggagcagaattcttgtcatctatgaggggtcctgagaaagacttgtcattttttttcctggagttcttcccattgaggtcctaggatttgcacaccactgtcccacaagagctttcctgcctaatgaaaggaggtcttgtggtgtgtgtctcctctcttctctatagttcccgagttggcccccattgcagcccccaccctgtgggtagtcttccagaagtgatgcagtggtgtgagatgccctgcaccttgttatttgggagactttgagagtcattcacttccatggtgactagtgtttgttttgcctgattttatattctgtgttgcatttctccccactccctgccctgctttaataaacagcaaaccaatatctaggaagaatgactgagggatagtattgggtattggccccatggcaggaacagccacttgcatctggtcccggtgccacactgcggtgcttggtgtggttgtggagcctgtccctgcgcgccttgctcccgttgagccacgctgtctggtgggtgattctctgccctgagccaccaccctggactggcccagtctccagagctggcacaccctgcctgttttctctttttagacacaacagccgcagtttggccagccactaagtcccaccagctgaggtccgaggaaagcggggtgactcatttcccttgtccagggcccgaggagagtgaggtgtccagcctgcaaagctattccagctccttggtgttggtttgcaataaattggtatttaagcaaaaaaaaaaaaaaaaaaaa

SEQ ID NO: 6 - DNA, 4235 bp, Homo Sapiens
cgtgatcatgaggggttgtgaagtgcttgccccatcagtagccatgtgtgcatgtgtaaataccatcctctgtgtgccctggaggctgtccttcagatagcatgtacaggtggcagcatagggcctgtccctactgagagtgcagggaactcagcaccgtcaactcctcgaccctgcaggtcagattatccttgtagaggccccctggatggcaccaagatcggccctggcaagtaggtgaccctgacttcagagcccttgcctgagggcctggcctggcagctctgctgttagaagcaggaggtgtgcagagggtggggagcagcccagcctctgtgatcttctccatggcaggatctcccagcaggtagagcagagccggagccaggtgcaggccattggagagaaggtctccttggcccaggccaagattgagaagatcaagggcagcaagaaggccatcaaggtagtccccatacccctgtgtcctgaggctactgggcagtccctccatttccccgtgcctctgaggctgcccagtctctgccctgctgcccacctgtaccttgagctttcttctcgcccaggcttccaactccaccctctcctgccaagcaatcctagccctctgagcctcttggggccccctcagacttgtccctgtgtccacaggtgttctccagtgccaagtaccctgctccagggcgcctgcaggaatatggctccatcttcacgggcgcccaggaccctggcctgcagagacgcccccgccacaggatccagagcaagcaccgccccctggacgagcgggccctgcaggtctgctggccgcgcatatagcctgtcacacaccaggaggactggatactggggaggagccggggccaccatagggttctgtcccccagaggaggctgactgggatgggatggcagctgattaggcccagcaccaaatattcaccatcccttggccatcctggccctctcaggagaagctgaaggactttcctgtgtgcgtgagcaccaagccggagcccgaggacgatgcagaagagggacttgggggtcttcccagcaacatcagctctgtcagctccttgctgctcttcaacaccaccgagaacctgtatggccagagggcagggccgaggggtgtgggcgggaggcccggcctggcttagtggggacccagggcatcagacacaggtacagcacataggccaggagccagggggtgacgggtggctcggctcgggaggcctgggaccccacagtgcacgctgtgcccctgatgatgtgggagaggaacatgggctcaggacagcgggtgtcagcttgcctgacccccatgtcgcctctgtaggtagaagaagtatgtcttcctggaccccctggctggtgctgtaacaaagacccatgtgatgctgggggcagagacagaggagaagctgtttgatgcccccttgtccatcagcaagagagagcagctggaacagcaggtgggaggggtgggacagaggtggagacaggtgcagtggcccagggccttgccagagctcctctccagtcaaggctgttgggccccttattccacccatgggaggtgcacacaaggtcttgttggctgcccctgcaggtccctgtcacctctcacatgtccctgcctaatcttgcaggtcccagagaactacttctatgtgccagacctgggccaggtgcctgagattgatgttccatcctacctgcctgacctgcccggcattgccaacgacctcatgtacattgccgacctgggccccggcattgccccctctgcccctggcaccattccagaactgcccaccttccacactgaggtagccgagcctctcaagacctacaagatggggtactaacaccacccccaccgcccccaccaccacccccagctcctgaggtgctggccagtgcacccccactcccaccctcaaccgcggcccctgtaggccaaggcgccaggcaggacgacagcagcagcagcgcgtctccttcaggtgggagcagctctttgaggccacctgatttctggcgtgctcagtgcactcgggtggattttctgtgggtttgttaagtggtcagaaattctcaattttttgaatagtttccatttcaaatatcttgttctacttggttcataaaatagtggttttcaaactgtagagctctggacttctcacttctagggcagagggagcctgaacaagtgaggctctgggttccccattcctaattaaaccaatggaaagaaggggtctaataacaaactacagcaacacatttttcatttcagcttcactgctgtgtctcccagtgtaaccctagcatccagaagtggcacaaaacccctctgctggctcgtgtgtgcaactgagactgtcagagcatggctagctcaggggtccagctctgcagggtgggggctagagaggaagcagggagtatctgcacacaggatgcccgcgctcaggtggttgcagaagtcagtgcccaggcccccacacacagtctccaaaggtccggcctccccagcgcagggctcctcgtttgaggggaggtgacttccctcccagcaggctcttggacacagtaagcttccccagccctgcctgagcagcctttcctccttgccctgttccccacctcccggctccagtccagggagctcccagggaagtggttgacccctccggtggctggccactctgctagagtccatccgccaagctgggggcatcggcaaggccaagctgcgcagcatgaaggagcgaaagctggagaagcagcagcagaaggagcaggagcaaggtgagcgggccctggagcttgcagtcggagggccttgggcaagatcgcctcctcccctccagccctgagtccaccgggtgctttctgcccaccccctgctcttgccagctggcccctgcttcccctagggcacatgctggaagccctgggccgccaccagaggtcctcagccctcctgcctgggctatggctccttcctggtttgggagccatagtggagctttcctctctaagctcacccagctcaaactgacaggagaatcttcttcgactgccaagagcggtccaaggcaatggtcagccactgcagcctcctgagatatttttagagactggacctgaggcctctggaggctactgatgatgcctgctgtgaacgcagacactggtgtgatgcgatgcctgcgcctgcagcggcagtgccctgggcactatggttttgagcttgtacccagcgctgcttttgccttgctctgtgaccccaggcaagctgcctcacctctctgggccagtttccccattgtacagtggtgctgcacaccctggccctggccccgaggtggctgggaggtggctcctcaaacagccgctgtctcatcagtgcccggtgctgggtcagggatcgactgaggctctgagctaactgggaaacacagtggccttggagggctggggagtgtcatgggggtggggacagggagtcaccggtcgcatgtgactgaactcttcaccccagtctgtggctttcccgttgcagtgagagccacgagccaaggtgggcacttgatgtcggatctcttcaacaagctggtcatgaggcgcaagggtaggaggcagggccgctgcccgccctgggccagcaccttgtaattctgtcctgcctttttcttcctgtatttaagtctccgggggctgggggaaccagggtttcccaccaaccaccctcactcagccttttccctccaggcatctctgggaaaggacctggggctggtgaggggcccggaggagcctttgcccgcgtgtcagactccatccctcctctgccgccaccgcagcagccacaggcagaggaggacgaggacgactgggaatcgtagggggctccatgacaccttcccccccagacccagacttgggccgttgctctgacatggacacagccaggacaagctgctcagacctacttccttgggagggggtgacggaaccagcactgtgtggagaccagcttcaaggagcggaaggctggcttgaggccacacagctggggcggggacttctgtctgcctgtgctccatggggggacggctccacccagcctgcgccactgtgttcttaagaggcttccagagaaaacggcacaccaatcaataaagaactgagcag

SEQ ID NO: 7 - DNA, 3932 bp, Homo Sapiens; identical to SEQ ID NO: 2

SEQ ID NO: 8 - DNA, 1257 bp, Homo Sapiens
ggcacgaggcgggccagcgacgggcaggacgccccgttcgcctagcgcgtgctcaggagttggtgtcctgcctgcgctcaggatgagggggaatctggccctggtgggcgttctaatcagcctggccttcctgtcactgctgccatctggacatcctcagccggctggcgatgacgcctgctctgtgcagatcctcgtccctggcctcaaaggggatgcgggagagaagggagacaaaggcgcccccggacggcctggaagagtcggccccacgggagaaaaaggagacatgggggacaaaggacagaaaggcagtgtgggtcgtcatggaaaaattggtcccattggctctaaaggtgagaaaggagattccggtgacataggaccccctggtcctaatggagaaccaggcctcccatgtgagtgcagccagctgcgcaaggccatcggggagatggacaaccaggtctctcagctgaccagcgagctcaagttcatcaagaatgctgtcgccggtgtgcgcgagacggagagcaagatctacctgctggtgaaggaggagaagcgctacgcggacgcccagctgtcctgccagggccgcgggggcacgctgagcatgcccaaggacgaggctgccaatggcctgatggccgcatacctggcgcaagccggcctggcccgtgtcttcatcggcatcaacgacctggagaaggagggcgccttcgtgtactctgaccactcccccatgcggaccttcaacaagtggcgcagcggtgagcccaacaatgcctacgacgaggaggactgcgtggagatggtggcctcgggcggctggaacgacgtggcctgccacaccaccatgtacttcatgtgtgagtttgacaaggagaacatgtgagcctcaggctggggctgcccattgggggccccacatgtccctgcagggttggcagggacagagcccagaccatggtgccagccagggagctgtccctctgtgaagggtggaggctcactgagtagagggctgttgtctaaactgagaaaatggcctatgcttaagaggaaaatgaaagtgttcctggggtgctgtctctgaagaagcagagtttcattacctgtattgtagccccaatgtcattatgtaattattacccagaattgctcttccataaagcttgtgcctttgtccaagctatacaataaaatctttaagtagtgcagtaaaaaaaaaaaaaaaaaaaaaaaaaaaaaaaa

SEQ ID NO: 9 - DNA, 3041 bp, Homo Sapiens
caatgctacattaacccattatgtaagaccaataaatgcagagccagcgtttcaagcacaggaaataccagcaggcagaatggccagtttgcttaagaatggtgagcctgaagctgagttacataaagaaaccacaggtccaggcactgctggccctcagtccaacaccacatcttctctaaaaggtgaacgcaaagccatccacacgctgcaagatgtgtcaacatgtgaaacaaaggagctattgaatgtcggggtttcctccctttgtgctggtccctaccaaaatacagcagacaccaaggaaaacctcagtaaagagcctttggcctcctttgtttcagaatcctttgatacttctgtttgtggaatagccacagagcacgtagaaattgagaacagtggggaggggctcagggctgaggctggttctgaaaccctaggcagagatggagaggtcggtgtgaattccgacatgcactatgaactctctggagattctgatctagacctgcttggtgattgtagaaatcccagactggatttggaggattcttatactttaagaggtagttacaccaggaaaaaagatgttcccacagatggctatgagtcgtcgttgaacttccacaacaacaaccaagaggactggggctgctctagccgggttccaggcatggagacgagcctccctcccgggcactggactgctgcggtaaagaaagaagagaagtgtgtgccgccttacgtccaaatccgagatctccacgggatcctcaggacttacgccaacttctctataacaaaagaactcaaagataccatgagaacttcacacggcctgaggaggcacccgagtttcagtgcaaactgtggcctgcccagctcctggacaagcacttggcaggtggcagacgacctcacccagaacactttagacctggagtatctgcgttttgcacataaactaaaacagaccataaagaatggggattctcagcattctgcctcctctgccaatgtctttccaaaggagtcaccaacccagatctccattggtgctttcccttcgacaaaaatctctgaggccccatttctgcatcctgcacctaggagcagaagcccccttctggtaacagctgtggagtcagatcccagaccacagggacagcccaggagaggctacacagccagcagtctggacatctcttcctcttggagagagagatgtagtcataatagagatcttagaaattctcaaagaaatcacactgtttcattccacctcaacaaactgaaatacaacagtactgtgaaggaatctcggaatgatatttcacttattctcaatgagtatgctgaattcaacaaggtgatgaagaatagcaaccaattcattttccaagacaaagagctaaatgatgtttctggagaagccactgctcaagagatgtatctgcctttcccaggacggtcagcctcctatgaagacataatcatagacgtgtgcaccaatttgcacgtcaaactaagaagtgttgtgaaagaggcttgtaaaagtaccttcctgttctaccttgtcgaaacagaagacaaatcattctttgtaagaacaaagaaccttctgaggaaaggaggccatacagaaattgaacctcagcacttctgtcaagctttccacagagagaatgatacactaatcatcatcatcagaaatgaagatatatcatcacatttgcatcagattccttctttgctgaagctgaagcatttccccagtgtcatctttgctggagtagacagccctggagatgttcttgatcacacctaccaagaactgtttcgtgcaggaggctttgtgatatcagatgacaagatactagaagctgtaacattagttcaactgaaggaaattatcaaaatcctggaaaaactaaatggaaatggaagatggaagtggttgcttcactacagggaaaataaaaagctaaaagaagatgaaagagtggattcaactgcacataagaagaacataatgttgaagtcatttcagagtgcaaatatcattgaattgcttcattatcaccagtgtgactctcgatcatcaacaaaagcagaaattctgaaatgtttgctaaacctgcaaattcagcatattgatgccaggtttgctgtcctcctaacagacaagcctactatccccagagaagtctttgaaaatagtggaatccttgttacagatgtaaataactttatagaaaacatagaaaaaatagcagctccatttaggagtagctattggtgactcaactacagcctgcctggatatggatgatgccaataaaaaattagtattttccctttggaaaacttgtgaacatgtgaatacacatgtgaagtcttacatttgaaaaaccaatgttctacaacttggaaagttttcattttttatattttgctgaaatatgtcacagtggcattgcagttgtctgttagctttgggttgcagtgctagatattgttttaaattattttcattttaaacaagatgccttctaagctattgagcttattaaaaataattttacatgtttacttagttggagcaaaaataagtctattttaacgaatagctttgtttttgctatgctaatgtctagaaaggcatacgatgctactattatgctctgttttaaaggttttacctacccttgtaaaaactataatcttaaatggttttatttgctgtttactacttatacatactactactataaaactattttttcctaaatggtacaaatttataaactatcatttttcacttacggtatttgtaaatactactactacaaaaatcagctttccgagaaagaaataatcatttatttatgatattgaaaatttctacagtaaacactcaaaaccaagcaaaaaacatttgtaagatacacggtatctatttggagcaacggtttttgtaactaatgtgtttcattttttaaataaagacaactaaaaataaaaaaaaaaaaaaaaaa

SEQ ID NO: 10 - DNA, 4484 bp, Homo Sapiens
tgcccaggaggagtaggagcaggagcagaagcagaagcggggtccggagctgcgcgcctacgcgggacctgtgtccgaaatgccggtgcgaggagaccgcgggtttccaccccggcgggagctgtcaggttggctccgcgccccaggcatggaagagctgatatgggaacagtacactgtgaccctacaaaaggattccaaaagaggatttggaattgcagtgtccggaggcagagacaacccccactttgaaaatggagaaacgtcaattgtcatttctgatgtgctcccgggtgggcctgctgatgggctgctccaagaaaatgacagagtggtcatggtcaatggcacccccatggaggatgtgcttcattcgtttgcagttcagcagctcagaaaaagtgggaaggtcgctgctattgtggtcaagaggccccggaaggtccaggtggccgcacttcaggccagccctcccctggatcaggatgaccgggcttttgaggtgatggacgagtttgatggcagaagtttccggagtggctacagcgagaggagccggctgaacagccatggggggcgcagccgcagctgggaggacagcccggaaagggggcgtccccatgagcgggcccggagccgggagcgggacctcagccgggaccggagccgtggccggagcctggagcggggcctggaccaagaccatgcgcgcacccgagaccgcagccgtggccggagcctggagcggggcctggaccacgactttgggccatcccgggaccgggaccgtgaccgcagccgcggccggagcattgaccaggactacgagcgagcctatcaccgggcctacgacccagactacgagcgggcctacagcccggagtacaggcgcggggcccgccacgatgcccgctctcggggaccccgaagccgcagccgcgagcacccgcactcacggagccccagccccgagcctagggggcggccggggcccatcggggtcctcctgatgaaaagcagagcgaacgaagagtatggtctccggcttgggagtcagatcttcgtaaaggaaatgacccgaacgggtctggcaactaaagatggcaaccttcacgaaggagacataattctcaagatcaatgggactgtaactgagaacatgtctttaacggatgctcgaaaattgatagaaaagtcaagaggaaaactacagctagtggtgttgagagacagccagcagaccctcatcaacatcccgtcattaaatgacagtgactcagaaatagaagatatttcagaaatagagtcaacccgatcattttctccagaggagagacgtcatcagtattctgattatgattatcattcctcaagtgagaagctgaaggaaaggccaagttccagagaggacacgccgagcagattgtccaggatgggtgcgacacccactccctttaagtccacaggggatattgcaggcacagttgtcccagagaccaacaaggaacccagataccaagaggaacccccagctcctcaaccaaaagcagccccgagaacttttcttcgtcctagtcctgaagatgaagcaatatatggccctaataccaaaatggtaaggttcaagaagggagacagcgtgggcctccggttggctggtggcaatgatgtcgggatatttgttgctggcattcaagaagggacctcggcggagcaggagggccttcaagaaggagaccagattctgaaggtgaacacacaggatttcagaggattagtgcgggaggatgccgttctctacctgttagaaatccctaaaggtgaaatggtgaccattttagctcagagccgagccgatgtgtatagagacatcctggcttgtggcagaggggattcgttttttataagaagccactttgaatgtgagaaggaaactccacagagcctggccttcaccagaggggaggtcttccgagtggtagacacactgtatgacggcaagctgggcaactggctggctgtgaggattgggaacgagttggagaaaggcttaatccccaacaagagcagagctgaacaaatggccagtgttcaaaatgcccagagagacaacgctggggaccgggcagatttctggagaatgcgtggccagaggtctggggtgaagaagaacctgaggaaaagtcgggaagacctcacagctgttgtgtctgtcagcaccaagttcccagcttatgagagggttttgctgcgagaagctggtttcaagagacctgtggtcttattcggccccatagctgatatagcaatggaaaaattggctaatgagttacctgactggtttcaaactgctaaaacggaaccaaaagatgcaggatctgagaaatccactggagtggtccggttaaataccgtgaggcaagttattgaacaggataagcatgcactactggatgtgactccgaaagctgtggacctgttgaattacacccagtggttctcaattgtgatttctttcacgccagactccagacaaggtgtcaacaccatgagacaaaggttagacccaacgtccaacaatagttctcgaaagttatttgatcacgccaacaagcttaaaaaaacgtgtgcacacctttttacagctacaatcaacctaaattcagccaatgatagctggtttggcagcttaaaggacactattcagcatcagcaaggagaagcggtttgggtctctgaaggaaagatggaagggatggatgatgaccccgaagaccgcatgtcctacttaactgccatgggcgcagactatctgagttgcgacagccgcctcatcagtgactttgaagacacggacggtgaaggaggcgcctacactgacaatgagctggatgagccagccgaggagccgctggtgtcgtccatcacccgctcctcggagccggtgcagcacgaggagagcataaggaaacccagcccagagccacgagctcagatgaggagggctgctagcagcgatcaacttagggacaatagcccgcccccagcattcaagccagagccgtccaaggccaaaacccagaacaaagaagaatcctatgacttctccaaatcctatgaatataagtcaaacccctctgccgttgctggtaatgaaactcctggggcatctaccaaaggttatcctcctcctgttgcagcaaaacctacctttgggcggtctatactgaagccctccactcccatccctcctcaagagggtgaggaggtgggagagagcagtgaggagcaagataatgctcccaaatcagtcctgggcaaagtcaaaatatttggagaagatggatcacaagggccagggttacaagagaatgcaggagctccaggaagcacagaatgcaaggatcgaaattgcccagaagcatcctgatatctatgcagttccaatcaaaacgcacaagccagaccctggcacgccccagcacacgagttccagaccccctgagccacagaaagctccttccagaccttatcaggataccagaggaagttatggcagtgatgccgaggaggaggagtaccgccagcagctgtcagaacactccaagcgcggttactatggccagtctgcccgataccgggacacagaattatagatgtctgagcacggactctcccaggcctgcctgcatggcatcagactagccactcctgccaggccgccgggatggttcttctccagttagaatgcaccatggagacgtggtgggactccagctcgtgtgtcctcatggagaacccaggggacagctggtgcaaattcagaactgagggctctgtttgtgggactgggttagaggagtctgtggctttttgttcagaattaagcagaacactgcagtcagatcctgttacttgcttcagtggaccgaaatctgtattctgtttgcgtacttgtaatatgtatattaagaagcaataactatttttcctcattaatagctgccttcaaggactgtttcagtgtgagtcagaatgtgaaaaaggaataaaaaatactgttgggctcaaactaaattcaaagaagtactttattgcaactcttttaagtgccttggatgagaagtgtcttaaattttcttcctttgaagctttaggcagagccataatggactaaaacattttgactaagtttttataccagcttaatagctgtagttttccctgcactgtgtcatcttttcaaggcatttgtctttgtaatattttccataaatttggactgtctatatcataactatacttgatagtttggctataagtgctcaatagcttgaagcccaagaagttggtatcgaaatttgttgtttgtttaaacccaagtgctgcacaaaagcagatacttgaggaaaacactatttccaaaagcacatgtattgacaacagttttataatttaataaaaaggaatacattgcaatccgt

SEQ ID NO: 11 - DNA, 3720 bp, Homo Sapiens
tgcccaggaggagtaggagcaggagcagaagcagaagcggggtccggagctgcgcgcctacgcgggacctgtgtccgaaatgccggtgcgaggagaccgcgggtttccaccccggcgggagctgtcaggttggctccgcgccccaggcatggaagagctgatatgggaacagtacactgtgaccctacaaaaggattccaaaagaggatttggaattgcagtgtccggaggcagagacaacccccactttgaaaatggagaaacgtcaattgtcatttctgatgtgctcccgggtgggcctgctgatgggctgctccaagaaaatgacagagtggtcatggtcaatggcacccccatggaggatgtgcttcattcgtttgcagttcagcagctcagaaaaagtgggaaggtcgctgctattgtggtcaagaggccccggaaggtccaggtggccgcacttcaggccagccctcccctggatcaggatgaccgggcttttgaggtgatggacgagtttgatggcagaagtttccggagtggctacagcgagaggagccggctgaacagccatggggggcgcagccgcagctgggaggacagcccggaaagggggcgtccccatgagcgggcccggagccgggagcgggacctcagccgggaccggagccgtggccggagcctggagcggggcctggaccaagaccatgcgcgcacccgagaccgcagccgtggccggagcctggagcggggcctggaccacgactttgggccatcccgggaccgggaccgtgaccgcagccgcggccggagcattgaccaggactacgagcgagcctatcaccgggcctacgacccagactacgagcgggcctacagcccggagtacaggcgcggggcccgccacgatgcccgctctcggggaccccgaagccgcagccgcgagcacccgcactcacggagccccagccccgagcctagggggcggccggggcccatcggggtcctcctgatgaaaagcagagcgaacgaagagtatggtctccggcttgggagtcagatcttcgtaaaggaaatgacccgaacgggtctggcaactaaagatggcaaccttcacgaaggagacataattctcaagatcaatgggactgtaactgagaacatgtctttaacggatgctcgaaaattgatagaaaagtcaagaggaaaactacagctagtggtgttgagagacagccagcagaccctcatcaacatcccgtcattaaatgacagtgactcagaaatagaagatatttcagaaatagagtcaacccgatcattttctccagaggagagacgtcatcagtattctgattatgattatcattcctcaagtgagaagctgaaggaaaggccaagttccagagaggacacgccgagcagattgtccaggatgggtgcgacacccactccctttaagtccacaggggatattgcaggcacagttgtcccagagaccaacaaggaacccagataccaagaggaacccccagctcctcaaccaaaagcagccccgagaacttttcttcgtcctagtcctgaagatgaagcaatatatggccctaataccaaaatggtaaggttcaagaagggagacagcgtgggcctccggttggctggtggcaatgatgtcgggatatttgttgctggcattcaagaagggacctcggcggagcaggagggccttcaagaaggagaccagattctgaaggtgaacacacaggatttcagaggattagtgcgggaggatgccgttctctacctgttagaaatccctaaaggtgaaatggtgaccattttagctcagagccgagccgatgtgtatagagacatcctggcttgtggcagaggggattcgttttttataagaagccactttgaatgtgagaaggaaactccacagagcctggccttcaccagaggggaggtcttccgagtggtagacacactgtatgacggcaagctgggcaactggctggctgtgaggattgggaacgagttggagaaaggcttaatccccaacaagagcagagctgaacaaatggccagtgttcaaaatgcccagagagacaacgctggggaccgggcagatttctggagaatgcgtggccagaggtctggggtgaagaagaacctgaggaaaagtcgggaagacctcacagctgttgtgtctgtcagcaccaagttcccagcttatgagagggttttgctgcgagaagctggtttcaagagacctgtggtcttattcggccccatagctgatatagcaatggaaaaattggctaatgagttacctgactggtttcaaactgctaaaacggaaccaaaagatgcaggatctgagaaatccactggagtggtccggttaaataccgtgaggcaagttattgaacaggataagcatgcactactggatgtgactccgaaagctgtggacctgttgaattacacccagtggttcccaattgtgatttttttcaacccagactccagacaaggtgtcaaaaccatgagacaaaggttaaatccaacgtccaacaaaagttctcgaaagttatttgatcaagccaacaagcttaaaaaaacgtgtgcacacctttttacagctacaatcaacctaaattcagccaatgatagctggtttggcagcttaaaggacactattcagcatcagcaaggagaagcggtttgggtctctgaaggaaagatggaagggatggatgatgaccccgaagaccgcatgtcctacttaaccgccatgggcgcggactatctgagttgcgacagccgcctcatcagtgactttgaagacacggacggtgaaggaggcgcctacactgacaatgagctggatgagccagccgaggagccgctggtgtcgtccatcacccgctcctcggagccggtgcagcacgaggaggtgaggcgaggcaggccacgggcaggaacaggagagcctggtgttttccttgcactctcgtggacagctgtgtgttcagggtgctgtggaaggcattcctaagggttggagcagatgacttccagggagtctctcgctttgagtccacgctggcatggttgcagtctgtggggaaagtggggcaggcaggtggacttcagaagagcttggaggggtcagcactccgcacacccatgccctcaggtgcgatggataaacagaatggctttaggtgccgtctgtccaaattaccagcggaaccttccttcccatgcagtattgttgtatgtacttgtaacctttgattaggtttctctctgtactcttagatgtccttgcttttcttccccatcctgcctttaacctttctaatcttgccaaagctcttgagtgtttccccatcagtttccttctctcttatatttcagttttttaattgagttcatgatcaaaccttcatctgatcacatcacatgtactgtgcatccactgtgattagatagcttatgggatccttgaaatcacattgacaggcactgtaaagtcacagccaagttagcaattattagttgcacctcagagaatgttggaataatgatctttgaagatgggattgttcatatatttggataattattgctgtggatttctctctagcattttagctcattccagtaaatgatttttttctttatgaaatagaactcccaaaaaaaaaaaaa

SEQ ID NO: 12 - DNA, 2820 bp, Homo Sapiens
caagcctggaagaactcgtcatgctctttgtagcgtggtgcttctgttgctcacaggacaacttgcctttgatgattttcaagagagttgtgctatgatgtggcaaaagtatgcaggaagcaggcggtcaatgcctctgggagcaaggatccttttccacggtgtgttctatgccgggggctttgccattgtgtattacctcattcaaaagtttcattccagggctttatattacaagttggcagtggagcagctgcagagccatcccgaggcacaggaagctctgggccctcctctcaacatccattatctcaagctcatcgacagggaaaacttcgtggacattgttgatgccaagttgaagattcctgtctctggatccaaatcagagggccttctctacgtccactcatccagaggtggcccctttcagaggtggcaccttgacgaggtctttttagagctcaaggatggtcagcagattcctgtgttcaagctcagtggggaaaacggtgatgaagtgaaaaaggagtagagacgacccagaagacccagcttgcttctagtccatccttccctcatctctaccatatggccactggggtggtggcccatctcagtgacagacactcctgcaacccagttttccagccaccagtgggatgatggtatgtgccagcacatggtaattttggtgtaattctaacttgggcacaacaaatgctatttgtcatttttaaactgaatccgaaagaaactcctattataaatttaagataatgtaatgtatttgaaagtgctttgtataaaaaagcacatgataaaaggaatcagaattaataaaatgtttgttgatctttaaaaaaaaaaaaaaaaaaactcgagactagttctgtctctccctcgtgccgaattcggcacgaggcagagcctcttctcgtctgtaggaacaccgccagggaggtcatggcagggcaggaccaaagggtcctgtggctctttttttttctcctgttctgcattcctgcccacacccccacccctccatttccttctgctctggaggcatcctccttcattggacaccacacagtttatttcacttctgacttcaaggttgtgaattcttcccatggcttaagtcctgggatacttctgcagtgaaaggaggtcttgtacctcttcctcagagtcagaagttctgagtacctttgccctattctgaaaagggctaggggctcctgctcccagctgccctcttcctttggcttccaattcagttccctctgccccgcatcctgcagacaggcgctcccgcagggggcccttgtggacctgcactggagtctgttgccttcactgagctgcctgtgctggccttgcatggtgcctgtagggggatttgctttgctgtgccattggggtacagctgctgctcttactctagaccaaaaagtcgggttgagtgactggtggcagggccacagatagagacagcggggagggtggctgaccctggcggccctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacataggtttggtaaaatgagtgctggaaattaactttctcccagtagtcttaggtcatgctcagtgaacttaaactttatccagatatggttttccttcagcctttctattccctttctagccagtgaaagacccgctgccctttgacctcagcccctccaagcccccaagtttaaaacgccaccccctgccggccctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacataggtttggtaaaatgagtgctggaaattaactttctcccagtagtcttaggtcatgctcagtgaacttaaactttatccagatatggttttccttcagcctttctattccctttctagccagtgaaagacccgctgccctttgacctcagcccctccaagcccccaagtttaaaacgccaccccctgccaccagaaaaaacagaaaaaaaaaaaaaaaaaaaactaaaacacccatctggtctgggcatcttcctttcctttttcactatgtatcctgttactgggcttaaacagctttcagagaagagatgtcatttctattaaatgctctttcagtagcgaactgagttcacacttgactaaggatattttccggactgtctgtcatcagcatccttagtgggtttccccatatttaaattggtagaggccagggatggtggctcacacctgtaatctcagtactttgggaggccaaggtaggtggattgcttgagctcagaagaccagcctgggcaacctggtgaaaccctgtctctactaaaaattcaagttagctagctgggcatggtgatgcacttctgtagtcccagctacttggagagggggtggtgctggggcagcaggatcgcttgaacccaggaggttgaggttgcagtgagccaagatggtaccagcctaggtgacaaagtgacaccctgtctcaaaaaagaaaccaaacaaaca

SEQ ID NO: 13 - DNA, 2802 bp, Homo Sapiens
agcggaggcggcggcggcggcggcggcggcagagggagtttccgctttgcactccaccccggtagcagctccgcggcagggacagcttcctccggacgcttggcgggcttcgctctcgccttacgacagcccggtcggatcatgggtttgcccagggggccggagggccagggtctcccggaggtggaaacaagagaagatgaagaacaaaatgtcaagttgactgaaattctggagctcttggttgcagctgggcatttcagggcaagaattaaaggcttatcaccctttgacaaggtagtaggaggaatgacttggtgtatcaccacttgcaactttgatgtagatgttgatttgctctttcaagaaaactctacgataggtcaaaaaatagctctgtcagaaaaaattgtctcggtcctgccaaggatgaaatgcccacaccagctggagccccaccagatccaggggatggattttattcacatatttcctgttgttcagtggctggtgaaacgagctatagaaacaaaagaagagatgggtgactatatccgctcctactctgtatcccagttccagaagacttacagtctccctgaggatgatgacttcataaagagaaaagaaaaggccatcaagacagttgtggacctctcagaagtgtacaagccccgtcggaaatacaaacgccaccagggagcagaggagctacttgatgaagaatctcgaatccatgctacacttttggaatatggcaggagatatggatttagctgccagagcaaaatggagaaggctgaggacaagaaaacggcacttccagcagggctgtcagctacagaaaaagctgatgcccacgaggaagatgagcttcgagcagctgaagagcagcgtattcagtcgctgatgaccaagatgaccgctatggcaaatgaggagagccgtctcaccgcaagctccgtgggccagattgtgggactctgctctgctgagatcaagcagattgtgtccgagtatgcagagaagcagtctgagctatcagctgaagaaagtccagaaaaattaggaacctcccagctacatcgccggaaagtcatttccttgaacaaacagattgcgcaaaagaccaaacatcttgaagagctgcgagcaagtcacaccagcctacaagccagatataatgaagccaagaaaacgctgacagagctgaagacttacagtgagaaactggacaaagagcaagcagccctcgagaagatagaatccaaagctgatccaagtatcctacagaacctgagagcacttgtagccatgaatgaaaatctgaaaagtcaagaacaggaatttaaagcacattgtcgagaggagatgacacgactacagcaagaaattgaaaacctgaaagctgagagagcaccacgtggagatgaaaagaccctctccagtggagagccgcctggtaccttgacctctgcaatgactcatgacgaagacctagacagacggtataatatggagaaagagaaactttacaagatacgtttactacaggctcgaagaaatcgagaaatagcaattttgcaccgcaagattgatgaagtccctagccgtgccgagctaatacagtatcagaagagatttattgaactctaccgccagatttcagcagtgcacaaagaaaccaagcagttcttcactttatataataccctggatgataaaaaggtttatttggaaaaagagattagtctgctgaactcaattcatgagaacttctcacaggccatggcctcccctgctgcccgggaccagtttttacgtcagatggaacagattgtggaaggaattaagcaaagtagaatgaagatggaaaagaaaaagcaagagaacaaaatgagaagagaccagttgaacgaccagtacttggagctgttagaaaagcagaggctatactttaagactgtgaaagagttcaaggaggagggccgcaagaacgagatgctgctgtccaaggtgaaagcgaaggcctcctgaacatccccagccgtggctgtatgtcattgattttacttttaagcaccgtatatcacctacaagatcatgaaatggttctgaaagcgacagtagagagatgcagttgtgatgatttcaacaacctggatgttttctttctcctctttgcttccattcatctctgttggctgctgttgatggagtcagacagtaaacacgtggcttggataacacccatcatcctatgaagaatatagggagtacttgttctctgttgattcaacttttatgtctccagtaacattgcgcttatgaaggtacctgtatttgtatggactctgaataaagaagaattcatttgtttagcaagtattagttcagcaaccactgagaaataagcactgaggaagattcagagacgtgtaaaacacagttcctactgcacaagtacccagcaggtggcccagggaggcagatacagcacacttgaccgcagaactgggctatccaagatgtttttcagtaaacagaaggcatttagctgaaatgatcagcccatgtagtgttggtcacttgggcctttcacctgccatggtaccttttgttcccagctcctccaggtgccagccagcaggcttggtggtgacagcaactggaacgaaagttcagtgttgttttaatttttatacgttactcaagttgatttctcagaaaattgaaaacagaccttgtgctgaggacacgtcaataaaaattataccttcccctacaaaaaaaaaaaaaaaa

SEQ ID NO: 14 - DNA, 900 bp, Homo Sapiens
ggaattccgtcgacggcagcggcggcggcgggtgggaaatggcggagtatctggcctccatcttcggcaccgagaaagacaaagtcaactgttcattttatttcaaaattggagcatgtcgtcatggagacaggtgctctcggttgcacaataaaccgacgtttagccagaccattgccctcttgaacatttaccgtaaccctcaaaactcttcccagtctgctgacggtttgcgctgtgccgtgagcgatgtggagatgcaggaacactatgatgagttttttgaggaggtttttacagaaatggaggagaagtatggggaagtagaggagatgaacgtctgtgacaacctgggagaccacctggtggggaacgtgtacgtcaagtttcgccgtgaggaagatgcggaaaaggctgtgattgacttgaataaccgttggtttaatggacagccgatccacgccgagctgtcacccgtgacggacttcagagaagcctgctgccgtcagtatgagatgggagaatgcacacgaggcggcttctgcaacttcatgcatttgaagcccatttccagagagctgcggcgggagctgtatggccgccgtcgcaagaagcatagatcaagatcccgatcccgggagcgtcgttctcggtctagagaccgtggtcgtggcggtggcggtggcggtggtggaggtggcggcggacgggagcgtgacaggaggcggtcgagagatcgtgaaagatctgggcgattctgagccatgccatttttaccttatgtctgctagaaagtgttgtagttgattgaccaaaccagttcataaggggaattttttaaaaaacaacaaaaaaaaaacatacaaagatgggtttctgaataaaaatttgtagtgataa

SEQ ID NO: 15 - DNA, 4500 bp, Homo Sapiens
cgggtggttgagtggaagcggtcgccatgtccgcggggagcgcgacacatcctggagctggcgggcgccgcagcaaatgggaccaaccagctccagccccacttctcttcctcccgccagcggccccaggtggggaggtcaccagcagtgggggaagtcctgggggcaccacagctgctccttcaggagccttggatgctgctgctgctgtggctgccaagattaatgccatgctcatggcaaaagggaagctgaaaccaactcagaatgcttctgagaagcttcaggctcctggcaaaggcctaactagcaataaaagcaaggatgacctggtggtagctgaagtagaaattaatgatgtgcctctcacatgtaggaacttgctgactcgaggacagactcaagacgagatcagccgacttagtggggctgcagtatcaactcgagggaggttcatgacaactgaggaaaaagccaaagtgggaccaggggatcgtccattatatcttcatgttcagggccagacacgggaattagtggacagagctgtaaaccggatcaaagaaattatcaccaatggagtggtaaaagctgccacaggaacaagtccaacttttaatggtgcaacagtaactgtctatcaccagccagcacccatcgctcagttgtctccagctgttagccagaagcctcccttccagtcagggatgcattatgttcaagataaattatttgtgggtctagaacatgctgtacccacttttaatgtcaaggagaaggtggaaggtccaggctgctcctatttgcagcacattcagattgaaacaggtgccaaagtcttcctgcggggcaaaggttcaggctgcattgagccagcatctggccgagaagcttttgaacctatgtatatttacatcagtcaccccaaaccagaaggcctggctgctgccaagaagctttgtgagaatcttttgcaaacagttcatgctgaatactctagatttgtgaatcagattaatactgctgtacctttaccaggctatacacaaccctctgctataagtagtgtccctcctcaaccaccatattatccatccaatggctatcagtctggttaccctgttgttccccctcctcagcagccagttcaacctccctacggagtaccaagcatagtgccaccagctgtttcattagcacctggagtcttgccggcattacctactggagtcccacctgtgccaacacaatacccgataacacaagtgcagcctccagctagcactggacagagtccgatgggtggtccttttattcctgctgctcctgtcaaaactgccttgcctgctggcccccagccccagccccagccccagcccccactcccaagtcagccccaggcacagaagagacgattcacagaggagctaccagatgaacgggaatctggactgcttggataccagcatggacccattcatatgactaatttaggtacaggcttctccagtcagaatgagattgaaggtgcaggatcgaagccagcaagttcctcaggcaaagagagagagagggacaggcagttgatgcctccaccagcctttccagtgactggaataaaaacagagtccgatgaaaggaatgggtctgggaccttaacagggagccatggtgagtgtgatatagctgggggaacaggggagtggctaagactggtctaaagctattagttttctcagccgggcgcagtggctcacgcctgtaatcccagcactttgggaggccgaggtgggcagatcacctaaggtcaggagttcaagaccagcttggccaacatagtgaaatcccatctctactaaaaatacaaaaactagcgggcatggtggtgggcgcctgtaattccagctactcagggggttgaggcaggagaatcgcttcaacctgggaggcagaggttgcagtgagccaagatcagaccactgccctccagcctgggcaatagagcaagactccatctcataaataaataaatacataaataaagctattaattttctaacctgatgttcattcaggtgtttaatccaacctctataatctgttggccagtgaaaatacttttgggctgggcacggtggctcacgcctgtaatcccagcactttgggaggccaaggtgggcggataacctgaggtcaggagtttgagaccagcgtggctaacacggtgaaaccccgtctctactaaaaatagaaaaattaagctgggcatggtggtgcatgcctgtaattccagcggcttggaaggctgaggcaggagaatcacttgaacttgggaggtggaggttgcagtgggccgagatcacaccactgcattccagcctgggcactagagtgagactctgtctcaaaaaaaaagaaagagaaagagaaaatagtttctaaaaaattgtatacagacaaccttttatttccaacaaacgtgtgccgagagagagagagagaaaatagttttaaaaaaattgtatacagacaaccttttgtttccaaccaacgtgtatctagaaaagagttagtcgacttattttatacatagcatcagtgaatagtaatgagtggtaggtcatttcaaaatcctgttgcctatattatgtgaataccaggaggtcatctgatacggacttaataaaggttgattttgctttatattgggagctgagccacacctccccttataactctattggtcagtaatggtcagtttgtggctgttaggaaaatgttgccttttagcattccagaactctaaatcctgtagaggtacatgggatattttattctttgcctgtactcataaaaatgaacagaagaaaatacgtttttttcttttcttaacttcttttcttttaactctttaaaaggtgaaatatcagccctcaagagactcacttgctaactttcctttttttctttttttttcttttttttgtgtttcttttttctttctctgttttcttacatggttctggtggattcacatttgctgatgctggtgctgtttttcgtgtgatcttcaacgtttttgggtgaccattgaccctgtgacctcaaaatggtgtccaactaaccacttaaaattaacatcttttttttaattaacgaatttatggtatttttttttttcccttggcggggatggggttggggttgttttttctctattctagattatccagccaagaagatgaaaactacagagaagggatttggcttggtggcttatgctgcagattcatctgatgaagaggaggaacatggaggtcataaaaatgcaagtagttttccacagggctggagtttgggataccaatatccttcatcacaaccacgagctaaacaacagatgccattctggatggctccctaggaaacagtggaacagagttttgaccctcagtgactcttcttagcaataatgcatgcatttgatttaacaagactctggggcctgtgctgggaaccatctggacctttgcagaagttagagattcagtgcccccctttcttaaaggggttccttaacaaccacaaaaatccttatttctgcagtggcatagaatctgttaaaatttaattagaatcacaaatttatctcagaagctttttaacagttggtgaaatgtgcttgtccaacaaagcatcctaacagggtcgttcccatacacatttgacctggtcagccttttccaggtgaatagccccagttctgacataaagaaagttttatttgtattttactactgtttggtcaattttgatatataactggttacaaacagagccttactatttattagtggggaaatgattttaagaccgtccttttcagtatttaattctgacagatctgcatccctgttttgttttggattatttctgttttggaaaatgctgtctcatttaaaactgttggatatagctggatcctggataggaaaatgaaattattttttcattgtgttttttaattggggtgatccaaagctggcaccttcaggcacattggtctcatagccattactgtttttattgcccttctaagatcctgtcttcagctgggtcagagaaaacttcttgactaaaactggtcagaactcatcacagaaatgaaatacagtggtctctctctcccagaactggttgcagctaaaacagagagatctgactgctggctataggattttggacttaatgactgaaattgcaaattgtcctttttcttggcattacagattttgccaaaataactttttgtatcaaatattgatgtgtgaaagtgaaggagctagtctgctgaaccaggaatagtttgagatattgaactgtcatttttgcacatttgaatactttgcaggctggctttgtataaacttatcctctggtttcctatatgttgtaaatatttagaccataatttcattataaataaatctataaatattc

SEQ ID NO: 16 - DNA, 1886 bp, Homo Sapiens
tcgactgccaaagcaatgaagcttgcggccgcggccacagtcatggcctttccccctggtgctcttcatcctttaccaaagagacaagcacttgaaaaaagcaatggtaccagcgcggtctttaaccccagcgtcttgcactaccagcaggctctcaccagcgcacagttgcagcaacacgccgcgttcattccaacaggtatgtgcccttactgccctacgtcctgtgcccttctggtcatgtgctttcttctcatttctctaagctgtttggtggcatctagtttgcttttgaaggtataatacagtttgaaattcatcgttgtcctagctatctaaatgtatttaccttactttgaatgatagctaaagactgttaggattctaaagccaaatatttgatagattgaagagacagatttaacccatgagaaacagcagttagggcttttggtttcttgtatttgcacaagccctgtaaaattgtttatgtaaataagaccttttatgtgtgacaattgaaatttgtccttaactctgaatgacctaaaaatagcaattccagtaaatactaaccatttttttctatttctattcagagcactaaaacaatgaggctattcaaattaaagcaattctctactcatatttttatattcattctatctctttctccatccttctcaactttcaccaagttcacaagtatatagagctcttatcctcagtgtctaagccaatgcctgatactattacgtacgatgtgcattaactatgattccactaaaagatccattgtaatagtcatagaatcttagagtttaaaggactcttagtgatctcctcatccagctgattgttttacagatgagaaaactgaggccccctaaatgagaagtgactttccaaggtgccacaactaatgagaaaaagaactgagtttccctgtgaccaaacccatttacatcacattctaccacctgggcccgcctatatatacacattccacagagttctcctgaaaaaaaaaaaaagcagataaaagtgaatttttaaataactgaccccaaaaagtcagataaaagtaaaaaaacaaaagtataaatcatgtcatccctcccccatttgcaccgacatctctaaccacagacacacacacgcacaccatacgcaaagatagtcaccataattgaccatgtttttcaccttttagtcaatgttagaagcaaggggtaacttaagtcctggtgggaagaccatccattgagttctttgaaagtcaacatttttcagcccacgatagtgaaatgaaagtaaatataaatgaataacaattctaacaaaaagagttttttgattcaaatccattagtttgaacttttcgagcttattatccatttccttaaatcccatagcttatcagagttaacatcagagggaggtaaaatatttctgtgatattctttgtataaaatctacactttgaaatggattagtaacctgtgaacaatacatattttagttaacatataaattatgtgagcaaagtggttttcagtgtttttttcttattttagttttgaacctgtcttaaactcacagacttgtagaagaaatctctaattcagtatttattaggagttcacttttgccctattacagccttaattagtgacatcccagtgctgttacagcatagcagtgtcttaatatgtaatctaattgaaataacacatttgtaaaataattactagaaggtaaacttacgttaatgtcctgtgtggtttctacaaagtgtgtcattgtagacctcttggccactagatattttaagataaaaaaaaaaaaaaatcgacgcggccgcgaatttagtagtagtagtaggc

SEQ ID NO: 17 - DNA, 1042 bp, Homo Sapiens
  misc_feature (854)..(868): n is a, c, g, or t
cggcacgagggctgctaagaaggcagacagcaccaagcgctaaatgagatggggcacctggtgctcttctgtgctactggtaggggtgcagcagagtggtcagtctggacagtagctgacatcacgtgacccaacacacgcattcctggctacttaccaaggagaatagaaagcaggcagatctctacagcagctctctacctgattgcaaaacaatggaaatgcccacatgtccacaaacaagtgtgtggtctgcctgtgccatgaagcacagtgtggctgagcgtcaagagtccccacactcaaaggaggcagcagatacagggctgcacactgtgtgattccacacatgtgacattctggacacggacatgctggatggcaaaacgagcatcgggctgagaggactgctgagaaggggaacggggctgctgggatgtgggttgattgtagcagtagctcatggagatgtgacctcaaaagagtgatttttactatgtgcatactatacctccacaaacttgactttaaaaaaataaaatattcacagaaaaaaacaaaaacaaatgtaaaaccatcagactactttatcagaggtgttatttttagatagaggtctttgaactccatcctaggaacattgtacccatgtcctcccagaactgcatcttgcactgggtgtcggaagacagccctgcaagacctgtatgctctgtaccattcagtggtttttaaggttaactaccagaagtcatatctgaggcctcccagaagcattactctaaggaaagtagttaaatgtggacagtgacagcagaaacatttacacattaaaccagtttatagaacatgannnnnnnnnnnnnnnaaagaagcttgtcagctcaatgacttacgaggcgtgggccattaaaaaaaaaggtctggagtttgggaaggagaaaggaatggggatgtgcagctcaagagtgtgatttttactatgtgcatagtatacagtgtggagacttgactttaggaaagtaaaatattcacagaaaaa

SEQ ID NO: 18 - DNA, 740 bp, Homo Sapiens
caacttcacggacgcattcaagaccatgctatcatgggaaatctggttatgttgtaatttttaatataattaaggtaaagcttaaatgtgctgttacgtgatttccttttaaagtttaaggttatctacctttgatattctctgtagatattagttgaacatagttctcaccaaagttagctatccaaattcaggaaaagcaaaactatttttccttttctttaaaaagaaaactttgattcatttactagattgtaaacttttttttaacttcaaaaataataaaagggtatgcagggaaaaatcttcctctcacctgtcagagctactttttaaatatgaaataagagaaaacaagtagctgcttataaggtgatgtgattacacttataaaagatgaatttagaaaacaacattcattgtctaatttaaatggtcaatagaatctttattttctttctccataagacatccagcttcacagcttcatgtgctacctagaactgatgatgccacaaatccttaaatgtcctaaatggtactgttaagtgaatcgtgcaattagaattttcacccaaacagaagggaaactgattttagatgtgattgggcttcttgaggacatttctgtggtctcgttttattgtttttttttttagctttgttactatcttaaattctttggttatcagcctagcactaaatgacctttaattaaaaaaaaaaaaaaaatcgtgccg

SEQ ID NO: 19 - DNA, 840 bp, Homo Sapiens
aatagaatgaatccaatttcttgccttgggttactgactctttcaattgtaactaagtacaatagcagttaagctcaagctgtaatagtagagctcagtggaagctaaaccaggcacagtaactgacaccatgtaggttgattatattttgcatctccctgcaagtctgttttatgttatttatagcttcctattcgtgtagacaccagcagtaaactggggaatatttgtggcaggaatttctaagaacaacctttagcatcatctcaggccctgatccatttccttttccacaaaattgtttgagattatatcgtatgtgttacagaaagaatgtttttctgtatgctcgaaactgtatactaaagtaaaataataaagttaaccagaattatccatggggaacaattccaattaaaataaaatgccagtatctggtaaaacctggtagtaatgctttttgtggtgatatccaggtaatgattagatgcagtaaacccgggtagtagggaagaagagagatgtggggacaagcagcccgaataccttgctggcatagcagctgcctacctgcacccggagacctgagcagatattactagggtattatttgacagccagcttagcagtcaagaaggacattgatttggggtagcatggcagaccacttcattggggctgaagacctgcatttattgatcacttactacatgccacgtatttcgtttaggatatatatgtgtgcatgtgtataattttaaaatataccccacggtagaggcagagctgttggcagtgagccgagatcgcgccactgcattccagcctgagcgacagagc

SEQ ID NO: 20 - DNA, 1012 bp, Homo Sapiens
tgcgctactttttttgagcctgggcgacagattgagactccgtctcaaaaaaaagaaaaaaaaaagaatgctttcatcagcaaaacattgtaacattccctttacttgagggcgtccacaataccgtaaggttgcgtgaactgtcctactgaatcttcatggttgcttggattttaatcacatcagaagaatttgagagcataccatggctggcagtccataaaagactagttaggaacatcagcttttaatcatcgaccctgctttcaggtttcattttaaacttatagaagaggggaagacatcagtgtgcttatttggcctttactctaaatcttaaaaggaagaaaattttaatatttcttagtttgagcccaggtgcggtgtctcacgcctgtaatcacagcactttgggaggccaaggcaggcggatcacttgaggtcaggagttcaagaccagcctgcaacgtggtgaaaccctgtctgtactaaaaattaaaaaaaaaaaaaaaaaaattagccgggcgtggtggcagtcgcctgtagtcccagcaactccagaggctgagacaggagaatcgcttgaaccccagaggtggaggttgcagtgagctgagatggtgccactgcactccagccgtgggcgacagagccagactgcatcttgtgggtgtaaaaaaaaaaatttgtagtttgagagtcaactttttcctcacagctttctgaaaatgtggccctttggatgctgataaaagctggtggtgattttaacaccttagtagccagaatcgagactgtcatggggcacttttaaaatctcaccacgatttgactcccattcacaaggtagccattggggctcagtctccctgaatgctcctgcaaaagtgcagtctgccaaggttttctctagaataatctcggtgtgtgttcactgtaacagttctgagttacacccagagttcattcggttaacattgttcctaccaggcaagacttctggtgttagaag

SEQ ID NO: 21 - DNA, 720 bp, Homo Sapiens
catgattacggattttaatccgcctcattatagggaatttggccctcgaggccaagaattcggcccccaggcacagaagagacgattcacagaggagctaccagatgaacgggaatttggactgcttggataccaggttaaataaaataccctgttttcctatcttcaccttattcttctactatattctccctttaaaaaagataaattcacatcattctcccagtactaggatttctgctttctggaattcattttggttaggttttttatcctattcaacagactcttgaaagcctctgagagttcttactttcttatacatctcactcaaagctcttgatctaccagtatgtggtttgtatttaaaaccttggctttcagtggtgctctctcttttaccctccacctaaaaaagagagtgatatctccctccagtctccccacccctcaagactgctagaaaaggagtgattctgtacatgtaattgtaaagttagccactaaagttaaaaagattcttaatttgtagttttggtgcaattttatcagaagtacctttccattttgccagaatccttgaatcattctttaaaccaaagcatttttttatagtttctagctaggtttatagaaactagtggagctatgggcagtcagttaaaaacaggccatagatagcataatgaattataacacccctgtccaagtcctatagag

SEQ ID NO: 22 - protein, 300 aa, Homo Sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 23 - protein, 235 aa, Homo Sapiens
MQQRGAAGSRGCALFPLLGVLFFQGVYIVFSLEIRADAHVRGYVGEKIKLKCTFKSTSDVTDKLTIDWTYRPPSSSHTVSIFHYQSFQYPTTAGTFRDRISWVGNVYKGDASISISNPTIKDNGTFSCAVKNPPDVHHNIPMTELTVTERGFGTMLSSVALLSILVFVPSAVVVALLLVRMGRKAAGLKKRSRSGYKKSSIEVSDDTDQEEEEACMARLCVRCAECLDSDYEETY

SEQ ID NO: 24 - protein, 360 aa, Homo Sapiens
MALNVAPVRDTKWLTLEVCRQFQRGTCSRSDEECKFAHPPKSCQVENGRVIACFDSLKGRCSRENCKYLHPPTHLKTQLEINGRNNLIQQKTAAAMLAQQMQFMFPGTPLHPVPTFPVGPAIGTNTAISFAPYLAPVTPGVGLVPTEILPTTPVIVPGSPPVTVPGSTATQKLLRTDKLEVCREFQRGNCARGETDCRFAHPADSTMIDTSDNTVTVCMDYIKGRCMREKCKYFHPPAHLQAKIKAAQHQANQAAVAAQAAAAAATVMAFPPGALHPLPKRQALEKSNGTSAVFNPSVLHYQQALTSAQLQQHAAFIPTGSVLCMTPATSIVPMMHSATSATVSAATTPATSVPFAATAT

SEQ ID NO: 25 - protein, 519 aa, Homo Sapiens
MASSPAVDVSCRRREKRRQLDARRSKCRIRLGGHMEQWCLLKERLGFSLHSQLAKFLLDRYTSSGCVLCAGPEPLPPKGLQYLVLLSHAHSRECSLVPGLRGPGGQDGGLVWECSAGHTFSWGPSLSPTPSEAPKPASLPHTTRRSWCSEATSGQELADLESEHDERTQEARLPRRVGPPPETFPPPGEEEGEEEEDNDEDEEEMLSDASLWTYSSSPDDSEPDAPRLLPSPVTCTPKEGETPPAPAALSSPLAVPALSASSLSSRAPPPAEVRVQPQLSRTPQAAQQTEALASTGSQAQSAPTPAWDEDTAQIGPKRIRKAAKRELMPCDFPGCGRIFSNRQYLNHHKKYQHIHQKSFSCPEPACGKSFNFKKHLKEHMKLHSDTRDYICEFCARSFRTSSNLVIHRRIHTGEKPLQCEICGFTCRQKASLNWHQRKHAETVAALRFPCEFCGKRFEKPDSVAAHRSKSHPALLLAPQESPSGPLEPCPSISAPGPLGSSEGSRPSASPQAPTLLPQQ

SEQ ID NO: 26 - protein, 220 aa, Homo Sapiens
MAHGSQEAEAPGAVAGAAEVPREPPILPRIQEQFQKNPDSYNGAVRENYTWSQDYTDLEVRVPVPKHVVKGKQVSVALSSSSIRVAMLEENGERVLMEGKLTHKINTESSLWSLEPGKCVLVNLSKVGEYWWNAILEGEEPIDIDKINKERSMATVDEEEQAVLDRLTFDYHQKLQGKPQSHELKVHEMLKKGWDAEGSPFRGQRFDPAMFNISPGAVQF

SEQ ID NO: 27 - protein, 157 aa, Homo Sapiens
MLGAETEEKLFDAPLSISKREQLEQQVGGVGQRWRQVQWPRALPELLSSQGCWAPYSTHGRCTQGLVGCPCRSLSPLTCPCLILQVPENYFYVPDLGQVPEIDVPSYLPDLPGIANDLMYIADLGPGIAPSAPGTIPELPTFHTEVAEPLKTYKMGY

SEQ ID NO: 28 - protein, 219 aa, Homo Sapiens
MQQRGAAGSRGCALFPLLGVLFFQGVYIVFSLEIRADAHVRGYVGEKIKLKCTFKSTSDVTDKLTIDWTYRPPSSSHTVSIFHYQSFQYPTTAGTFRDRISWVGNVYKGDASISISNPTIKDNGTFSCAVKNPPDVHHNIPMTELTVTERGFGTMLSSVALLSILVFVPSAVVVALLLVRMGRKAAGLKKRSRSGYKKSSIEVSDDTDQEEEEACMARL

SEQ ID NO: 29 - protein, 271 aa, Homo Sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 30 - protein, 741 aa, Homo Sapiens
MASLLKNGEPEAELHKETTGPGTAGPQSNTTSSLKGERKAIHTLQDVSTCETKELLNVGVSSLCAGPYQNTADTKENLSKEPLASFVSESFDTSVCGIATEHVEIENSGEGLRAEAGSETLGRDGEVGVNSDMHYELSGDSDLDLLGDCRNPRLDLEDSYTLRGSYTRKKDVPTDGYESSLNFHNNNQEDWGCSSRVPGMETSLPPGHWTAAVKKEEKCVPPYVQIRDLHGILRTYANFSITKELKDTMRTSHGLRRHPSFSANCGLPSSWTSTWQVADDLTQNTLDLEYLRFAHKLKQTIKNGDSQHSASSANVFPKESPTQISIGAFPSTKISEAPFLHPAPRSRSPLLVTAVESDPRPQGQPRRGYTASSLDISSSWRERCSHNRDLRNSQRNHTVSFHLNKLKYNSTVKESRNDISLILNEYAEFNKVMKNSNQFIFQDKELNDVSGEATAQEMYLPFPGRSASYEDIIIDVCTNLHVKLRSVVKEACKSTFLFYLVETEDKSFFVRTKNLLRKGGHTEIEPQHFCQAFHRENDTLIIIIRNEDISSHLHQIPSLLKLKHFPSVIFAGVDSPGDVLDHTYQELFRAGGFVISDDKILEAVTLVQLKEIIKILEKLNGNGRWKWLLHYRENKKLKEDERVDSTAHKKNIMLKSFQSANIIELLHYHQCDSRSSTKAEILKCLLNLQIQHIDARFAVLLTDKPTIPREVFENSGILVTDVNNFIENIEKIAAPFRSSYW

SEQ ID NO: 31 - protein, 1116 aa, Homo Sapiens
MPVRGDRGFPPRRELSGWLRAPGMEELIWEQYTVTLQKDSKRGFGIAVSGGRDNPHFENGETSIVISDVLPGGPADGLLQENDRVVMVNGTPMEDVLHSFAVQQLRKSGKVAAIVVKRPRKVQVAALQASPPLDQDDRAFEVMDEFDGRSFRSGYSERSRLNSHGGRSRSWEDSPERGRPHERARSRERDLSRDRSRGRSLERGLDQDHARTRDRSRGRSLERGLDHDFGPSRDRDRDRSRGRSIDQDYERAYHRAYDPDYERAYSPEYRRGARHDARSRGPRSRSREHPHSRSPSPEPRGRPGPIGVLLMKSRANEEYGLRLGSQIFVKEMTRTGLATKDGNLHEGDIILKINGTVTENMSLTDARKLIEKSRGKLQLVVLRDSQQTLINIPSLNDSDSEIEDISEIESTRSFSPEERRHQYSDYDYHSSSEKLKERPSSREDTPSRLSRMGATPTPFKSTGDIAGTVVPETNKEPRYQEEPPAPQPKAAPRTFLRPSPEDEAIYGPNTKMVRFKKGDSVGLRLAGGNDVGIFVAGIQEGTSAEQEGLQEGDQILKVNTQDFRGLVREDAVLYLLEIPKGEMVTILAQSRADVYRDILACGRGDSFFIRSHFECEKETPQSLAFTRGEVFRVVDTLYDGKLGNWLAVRIGNELEKGLIPNKSRAEQMASVQNAQRDNAGDRADFWRMRGQRSGVKKNLRKSREDLTAVVSVSTKFPAYERVLLREAGFKRPVVLFGPIADIAMEKLANELPDWFQTAKTEPKDAGSEKSTGVVRLNTVRQVIEQDKHALLDVTPKAVDLLNYTQWFSIVISFTPDSRQGVNTMRQRLDPTSNNSSRKLFDHANKLKKTCAHLFTATINLNSANDSWFGSLKDTIQHQQGEAVWVSEGKMEGMDDDPEDRMSYLTAMGADYLSCDSRLISDFEDTDGEGGAYTDNELDEPAEEPLVSSITRSSEPVQHEESIRKPSPEPRAQMRRAASSDQLRDNSPPPAFKPEPSKAKTQNKEESYDFSKSYEYKSNPSAVAGNETPGASTKGYPPPVAAKPTFGRSILKPSTPIPPQEGEEVGESSEEQDNAPKSVLGKVKIFGEDGSQGPGLQENAGAPGSTECKDRNCPEAS

SEQ ID NO: 32 - protein, 993 aa, Homo Sapiens
MPVRGDRGFPPRRELSGWLRAPGMEELIWEQYTVTLQKDSKRGFGIAVSGGRDNPHFENGETSIVISDVLPGGPADGLLQENDRVVMVNGTPMEDVLHSFAVQQLRKSGKVAAIVVKRPRKVQVAALQASPPLDQDDRAFEVMDEFDGRSFRSGYSERSRLNSHGGRSRSWEDSPERGRPHERARSRERDLSRDRSRGRSLERGLDQDHARTRDRSRGRSLERGLDHDFGPSRDRDRDRSRGRSIDQDYERAYHRAYDPDYERAYSPEYRRGARHDARSRGPRSRSREHPHSRSPSPEPRGRPGPIGVLLMKSRANEEYGLRLGSQIFVKEMTRTGLATKDGNLHEGDIILKINGTVTENMSLTDARKLIEKSRGKLQLVVLRDSQQTLINIPSLNDSDSEIEDISEIESTRSFSPEERRHQYSDYDYHSSSEKLKERPSSREDTPSRLSRMGATPTPFKSTGDIAGTVVPETNKEPRYQEEPPAPQPKAAPRTFLRPSPEDEAIYGPNTKMVRFKKGDSVGLRLAGGNDVGIFVAGIQEGTSAEQEGLQEGDQILKVNTQDFRGLVREDAVLYLLEIPKGEMVTILAQSRADVYRDILACGRGDSFFIRSHFECEKETPQSLAFTRGEVFRVVDTLYDGKLGNWLAVRIGNELEKGLIPNKSRAEQMASVQNAQRDNAGDRADFWRMRGQRSGVKKNLRKSREDLTAVVSVSTKFPAYERVLLREAGFKRPVVLFGPIADIAMEKLANELPDWFQTAKTEPKDAGSEKSTGVVRLNTVRQVIEQDKHALLDVTPKAVDLLNYTQWFPIVIFFNPDSRQGVKTMRQRLNPTSNKSSRKLFDQANKLKKTCAHLFTATINLNSANDSWFGSLKDTIQHQQGEAVWVSEGKMEGMDDDPEDRMSYLTAMGADYLSCDSRLISDFEDTDGEGGAYTDNELDEPAEEPLVSSITRSSEPVQHEEVRRGRPRAGTGEPGVFLALSWTAVCSGCCGRHS

SEQ ID NO: 33 - protein, 146 aa, Homo Sapiens
MMWQKYAGSRRSMPLGARILFHGVFYAGGFAIVYYLIQKFHSRALYYKLAVEQLQSHPEAQEALGPPLNIHYLKLIDRENFVDIVDAKLKIPVSGSKSEGLLYVHSSRGGPFQRWHLDEVFLELKDGQQIPVFKLSGENGDEVKKE

SEQ ID NO: 34 - protein, 575 aa, Homo Sapiens
MTWCITTCNFDVDVDLLFQENSTIGQKIALSEKIVSVLPRMKCPHQLEPHQIQGMDFIHIFPVVQWLVKRAIETKEEMGDYIRSYSVSQFQKTYSLPEDDDFIKRKEKAIKTVVDLSEVYKPRRKYKRHQGAEELLDEESRIHATLLEYGRRYGFSCQSKMEKAEDKKTALPAGLSATEKADAHEEDELRAAEEQRIQSLMTKMTAMANEESRLTASSVGQIVGLCSAEIKQIVSEYAEKQSELSAEESPEKLGTSQLHRRKVISLNKQIAQKTKHLEELRASHTSLQARYNEAKKTLTELKTYSEKLDKEQAALEKIESKADPSILQNLRALVAMNENLKSQEQEFKAHCREEMTRLQQEIENLKAERAPRGDEKTLSSGEPPGTLTSAMTHDEDLDRRYNMEKEKLYKIRLLQARRNREIAILHRKIDEVPSRAELIQYQKRFIELYRQISAVHKETKQFFTLYNTLDDKKVYLEKEISLLNSIHENFSQAMASPAARDQFLRQMEQIVEGIKQSRMKMEKKKQENKMRRDQLNDQYLELLEKQRLYFKTVKEFKEEGRKNEMLLSKVKAKAS

SEQ ID NO: 35 - protein, 240 aa, Homo Sapiens
MAEYLASIFGTEKDKVNCSFYFKIGACRHGDRCSRLHNKPTFSQTIALLNIYRNPQNSSQSADGLRCAVSDVEMQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 36 - protein, 564 aa, Homo Sapiens
MSAGSATHPGAGGRRSKWDQPAPAPLLFLPPAAPGGEVTSSGGSPGGTTAAPSGALDAAAAVAAKINAMLMAKGKLKPTQNASEKLQAPGKGLTSNKSKDDLVVAEVEINDVPLTCRNLLTRGQTQDEISRLSGAAVSTRGRFMTTEEKAKVGPGDRPLYLHVQGQTRELVDRAVNRIKEIITNGVVKAATGTSPTFNGATVTVYHQPAPIAQLSPAVSQKPPFQSGMHYVQDKLFVGLEHAVPTFNVKEKVEGPGCSYLQHIQIETGAKVFLRGKGSGCIEPASGREAFEPMYIYISHPKPEGLAAAKKLCENLLQTVHAEYSRFVNQINTAVPLPGYTQPSAISSVPPQPPYYPSNGYQSGYPVVPPPQQPVQPPYGVPSIVPPAVSLAPGVLPALPTGVPPVPTQYPITQVQPPASTGQSPMGGPFIPAAPVKTALPAGPQPQPQPQPPLPSQPQAQKRRFTEELPDERESGLLGYQHGPIHMTNLGTGFSSQNEIEGAGSKPASSSGKERERDRQLMPPPAFPVTGIKTESDERNGSGTLTGSHGECDIAGGTGEWLRLV